(19) United States
(12) Patent Application Publication
(10) Pub. No.: US 2012/0219506 A1
(43) Pub. Date: Aug. 30, 2012

(54) Catenae: Serosal Cancer Stem Cells

(75) Inventors: Malcolm A.S. Moore, New York, NY (US); Server A. Ertem, New York, NY (US)

(73) Assignee: SLOAN KETTERING INSTITUTE FOR CANCER RESEARCH, New York, NY (US)

(21) Appl. No.: 13/508,110

(22) PCT Filed: Nov. 5, 2010
(86) PCT No.: PCT/US10/55538

§ 371 Date: May 4, 2012

Related U.S. Application Data
(60) Provisional application No. 61/293,113, filed on Jan. 7, 2010.
(60) Provisional application No. 61/258,570, filed on Nov. 5, 2009.

(51) Int. Cl. A61K 49/00; A61K 38/46; C12Q 1/68; G01N 33/574; C12Q 1/34; C12Q 1/37; C12Q 1/02; C12Q 1/06; C12P 1/00; C12N 5/00; C12N 5/02; C12N 15/63; C07K 14/435; C07H 21/04; A61P 35/00; C07H 21/02; A61K 38/54

(52) U.S. Cl. 424/9.2; 424/94.3; 424/94.62; 424/94.67; 435/6.14; 435/7.1; 435/18; 435/23; 435/29; 435/39; 435/41; 435/325; 435/378; 435/320.1; 530/350; 536/23.5; 536/24.31; 536/24.33

(57) ABSTRACT

The present invention relates to a clonally pure population of serosal cancer stem cells (CSCs) as well as methods of producing and culturing the CSCs and uses thereof. The CSCs form catenae (free floating chains of cells) which have a glycocalyx coat of hyaluronan and proteoglycans. This discovery has lead to the development of methods of treating serosal and ovarian cancers by targeting removal or inhibition of glycocalyx formation, including combination therapies using chemotherapeutics in conjunction with glycocalyx inhibitors. The invention also provides drug screening assays for identifying compounds effective against these CSCs as well as other serosal cancer cells. Methods to use catena gene signatures, protein and surface antigens are provided for monitoring patient samples for the presence of serosal cancer stem cells.

This application claims priority of provisional applications U.S. Ser. No. 61/258,570, filed Nov. 5, 2009 and U.S. Ser. No. 61/293,113, filed Jan. 7, 2010, each of which is incorporated herein by reference in its entirety.

### FIELD OF THE INVENTION

The present invention relates to a clonally pure population of serosal cancer stem cells (CSCs) as well as methods of producing and culturing the CSCs and uses thereof. The CSCs form catenae (free floating chains of cells) which have a glycocalyx coat of hyaluronan and proteoglycans. This discovery has lead to the development of methods of treating serosal and ovarian cancers by targeting removal or inhibition of glycocalyx formation, including combination therapies using chemotherapeutics in conjunction with glycocalyx inhibitors. The invention also provides drug screening assays for identifying compounds effective against these CSCs as well as other serosal cancer cells. Methods to use catena gene signatures, protein and surface antigens are provided for monitoring patient samples for the presence of serosal cancer stem cells.

### BACKGROUND OF THE INVENTION

The cancer stem cell (CSC) hypothesis suggests that in cancer, either normal tissue stem cells become malignant or more differentiated tissue can be transformed and develop stem cell characteristics. Human CSCs are generally defined as a “rare” population of malignant cells that can undergo unlimited self-renewal with symmetric division capacity. These “tumor initiating cells” or cancer stem cells can regenerate all the components of the original tumor when serially transplanted.

The concept of cancer stem cells has had a major impact on our understanding of how to treat cancer. Unfortunately, unless CSCs can be eradicated, they may proliferate again and generate the cancer, leading to relapse. CSCs are thought to be particularly resistant to chemotherapy and radiation, making them particularly difficult to eliminate even with treatment that can efficiently destroy the bulk of the tumor and produce remission.

The CSC hypothesis depends on prospective purification of cells with tumor-initiating capacity, irrespective of frequency. The cancer stem cell hypothesis recognizes that the incidence of CSCs relative to more differentiated tumor cells can vary markedly from 0.001% to 100% depending on tumor type, stage of tumor development (e.g., metastatic vs. non-metastatic), or if studies were done on tumor cell lines selected from primary tumors, with high CSC content in the first place.

A number of in vitro assays, such as cloning in semi-solid medium, oncospheroid formation, limiting-dilution serial recloning, stromal colony formation, have been developed for CSCs. However, in vitro CSC assays are limited by the problem of an unknown and probably variable “plating efficiency” dependent on provision of, e.g., the appropriate combination and concentration of growth factors, morphogens and/or interactive niche components. The current “gold standard” for human CSCs is the tumor initiating limiting-dilution assay in immuno-deficient mice (Nude, SCID or NOD-SCID), however these recipients have innate immune resistance (Natural Killer (NK), macrophage). Furthermore, any in vivo assay has a “seeding efficiency” depending how efficient the cells are in localizing to their correct “niche.” If CSCs are injected into non-orthotopic sites (e.g., subcutaneously) lacking the appropriate “niche” or microenvironment (mesenchymal, endothelial), their numbers may be underestimated due to death or terminal differentiation. If injected intravenously, e.g., in metastatic models, the ability of CSCs to egress the vasculature and find appropriate niches may be determined by variable expression of homing receptors (e.g., integrins) and chemokine receptors (e.g., CXCR4), independent of the stem cell status of the cell. If the CSC is dependent on paracrine stimulation by growth factors or morphogens(e.g., IL-6, GM-CSF, M-CSF, IL-3 HGF), species specificity may exist. The existence of transit amplifying progenitor populations has been established in most tissues and such populations can generate billions of differentiated cells. Consequently, a primary in vivo assay for tumor development is not apriori a CSC assay unless re-passaging capacity can be demonstrated.

Ovarian cancer ranks fifth in cancer deaths among women and causes more deaths than any other gynecologic malignancy. It is estimated that in the United States 22,430 new cases will be diagnosed each year with 15,280 deaths [Jemal, 2008]. Ovarian carcinoma remains enigmatic in at least two important respects. First, the histological region of origin for this cancer remains obscure and second, an identifiable premalignant lesion that is generally recognized by cancer pathologists is yet to be defined. The majority (80%) of patients present with advanced stage disease with cancer cells throughout the abdominal cavity, leading directly to the high mortality (5 year survival rates 15-45%). In contrast, the survival rate for early stage disease, with malignancy confined to the ovary, is ˜95%. Given the discrepancy in survival outcomes between early- and late-stage diseases, strategies that would allow for the detection of ovarian cancer in its early stages would hold promise to significantly improve survival. Unfortunately, current screening methods for the detection of early stage ovarian cancer are inadequate.

The median overall survival for patients with advanced ovarian cancer has improved from approximately 1 year in 1975 to currently in excess of 3 years and for subsets having optimally debulked disease and treatment with taxane- and platinum-base combination chemotherapy, survival now exceeds 5 years [Ozols; Markman, 2003]. However the disease course is one of remission and relapse requiring intermittent re-treatment. Understanding the biology of CSCs and the mechanism by which such cells survive multiple rounds of chemotherapy to metastasize and regenerate tumors is important in the quest to find early stage detection methods and to eradicate ovarian cancer.

Opportunities to improve both overall survival and quality of life would include the development of novel therapies specifically designed to target the ovarian CSCs or other serosal CSCs. Eradicating cancer stem cells as well as differentiated cancer cells might increase the efficiency of therapy for ovarian or other serosal cancers, including metastatic serosal cancer.

The presence of cancer cells in effusions within the serosal (peritoneal, pleural, and pericardial) cavities is a clinical manifestation of advanced stage cancer and is associated with poor survival. Tumor cells in effusions most frequently originate from primary carcinomas of the ovary, breast, and lung, and from malignant mesothelioma, a native tumor of this anatomic site [Di Maria, 2007; Davidson, 2007]. Unlike the majority of solid tumors, particularly at the primary site, cancer cells in effusions are not amenable to surgical removal and failure in their eradication is one of the main causes of treatment failure [Davidson, 2007].

Formation of tumor spheroids (also referred to as oncospheroids) is a mechanism for tumor cells to adapt to grow in exudative fluids. Tumor spheroids are found in pleural, pericardial effusions and ascites samples from patients with serosal cancers. The pathophysiological relevance of tumor spheroids is best illustrated in ovarian cancer since a significant proportion of cancer cells in peritoneal ascites exist as spheroids. Advances in cancer therapy will depend on identification of novel therapeutic agents that can target CSCs that exists as individual entities or as these multicellular spheroids. Furthermore, screening systems will allow development of compounds toxic to both cycling stem cells and CSCs in a quiescent GO state.

While there have been some recent reports of isolation of subpopulations of cells from ovarian cancer that appeared to be enriched for cells capable of initiating tumors when transplanted into immunodeficient mice [Szotek, 2006; Zhang, 2008; Bapat, 2005], there have been no reports of clonally pure cells that can be maintained in their stem cell state in a tissue culture system. The lack of an in vitro system to maintain and expand clonally pure cells without differentiation has hindered the gene expression profiling and proteomics analysis of serosal cancer stem cells. Furthermore, lack of an in vitro culture system for CSC expansion has slowed down the development of high throughput drug screenings with potential to identify novel compounds that specifically target CSCs.

### SUMMARY OF THE INVENTION

In one aspect, the present invention provides a method to produce serosal cancer stem cells which comprises (a) injecting an immunocompromised, non-human mammal intraperitoneally with serosal epithelial tumor cells in an amount and under conditions to produce an intraperitoneal (ip) tumor; (b) harvesting ascites from an ip tumor-bearing, non-human mammal; (c) fractionating the ascites into a first fraction comprising serosal catena and leukocytes and a second fraction comprising serosal spheroids; (d) removing the leukocytes from said first fraction to obtain a catena-enriched fraction; and (e) culturing the catena-enriched fraction for a time and under conditions to produce adherent mesenchymal cells and a suspension of serosal catena enriched for serosal cancer stem cells. This method can further comprise (f) collecting the suspension of serosal catenae; (g) separating the serosal catena from any serosal spheroids that may have formed; and (h) serially passaging these catenae in suspension for a time and under conditions to produce a stable culture of free-floating serosal catena comprising from at least 50 to 100% serosal cancer stem cells.

In another aspect, the invention is directed to a method to produce serosal cancer stem cells which comprises (a) injecting an immunocompromised, non-human mammal intraperitoneally with serosal epithelial tumor cells in an amount and under conditions to produce an intraperitoneal (ip) tumor; (b) harvesting ascites from an ip tumor-bearing, non-human mammal; (c) fractionating the ascites into a first ascites fraction comprising serosal catena and leukocytes and a second ascites fraction comprising serosal spheroids; (d) culturing the second fraction for a time and under conditions to produce adherent mesenchymal cells and a suspension culture of free-floating catena and tumor spheroids; and (e) fractionating the suspension culture into a first culture fraction comprising free-floating catena enriched for serosal cancer stem cells and a second culture fraction comprising free-floating tumor spheroids enriched for serosal cancer stem cells. This method can further comprises (f) culturing said second culture fraction for a time and under conditions to produce a further suspension culture of free-floating catena and tumor spheroids; (g) fractionating said further suspension culture into free-floating catena and tumor spheroid fractions; and (h) repeating steps (f) and (g) with the free-floating spheroid fraction for a time and under conditions to produce a (stable) suspension culture of free-floating tumor spheroids comprising at least 10-30% serosal cancer stem cells (as determined by in vitro recloning capacity).

In yet another aspect, the invention is directed to a method to isolate serosal catenae which comprises (a) injecting an immunocompromised, non-human mammal intraperitoneally with serosal epithelial tumor cells in an amount and under conditions to produce an intraperitoneal (ip) tumor; (b) harvesting ascites from an ip tumor-bearing, non-human mammal; (c) fractionating the ascites into a first fraction comprising serosal catena and leukocytes and a second fraction comprising serosal spheroids; and (d) removing the leukocytes from said first fraction to obtain a catena-enriched fraction. In accordance with the invention, spheroids can be isolated by (a) injecting an immunocompromised, non-human mammal intraperitoneally with ovarian epithelial tumor cells in an amount and under conditions to produce an intraperitoneal (ip) tumor; (b) harvesting ascites from an ip tumor-bearing, non-human mammal; (c) fractionating the ascites into a first fraction comprising serosal catena and leukocytes and a second fraction comprising serosal spheroids; and (d) isolating the serosal spheroids.

In the foregoing methods of the invention, one can induce intraperitoneal inflammation, prior to, concurrent with or after injection of the cells using methods known in the art. Immunocompromised non-human mammals for use in these methods include, mice lacking T cells, B cells and/or Natural Killer (NK) cells. In preferred embodiments, useful mice include but are not limited to NOD/SCID mice, NSG mice and NOG mice. As shown in the Examples, fractionation of the ascites is conveniently accomplished by filtering it through a 30-60 μm filter, and even more preferably through a 40 μm filter, to obtain a flow-through fraction that contains the catenae and leukocytes and a retained fraction that contains the larger, spheroids.

In using these methods, one obtains, in the catenae, isolated clonally pure serosal cancer stem cells. These clonally pure, serosal cancer stem cells are a self-renewing population of cells which comprise symmetrically dividing, free-floating chains of cells with from about three to four (3-4) to about seventy-two (72) cells, or more. The chains are surrounded by a glycocalyx of hyaluronan, collagen and other extracellular components. These cells are E-cadherin negative, have increased engraftment potential relative to serosal epithelial tumor cells and have at least 50% recloning capacity in vitro. In certain embodiments, the serosal cells are ovarian cells. These free floating chains are termed catenae or serosal cancer stem cells.

Another aspect of the invention provides methods to screen a test compound for anti-proliferative effects by (a) culturing any one of dissociated serosal catena cells, dissociated serosal spheroid cells or dissociated serosal cancer adherent cells, all of which cells are capable of fluorescence or luminescence; (b) contacting the cells with a test compound; (c) detecting whether the cells proliferate in response by detecting the fluorescence or luminescence emitted by the cultures; and (d) determining whether the test compound has inhibited proliferation of the catenae, spheroids or adherent cells. In some embodiments, the method includes determining whether the test compound differentially inhibits proliferation of the catenae relative to the spheroids or adherent cells. Additionally, these methods can be adapted to screen a compound for its morphological effects on serosal cancer stem cells by having step (c) be detecting morphological changes (e.g., such as changes from catena to spheroid, spheroid to catena, catena to epithelial monolayer, catena to mesenchymal monolayer, spheroid to epithelial monolayer, spheroid to mesenchymal monolayer, or alterations in cell morphological shape, arrest at particular cell cycle stages, and the like). These methods can be readily adapted for high throughput screening (HTS) by growing the cells in 384- or 1536-well plates, for example, and conducting the assays using robotics systems for manipulating reagents, and collecting and analyzing the data. Such systems are known in the art.

In conducting screening assays with test compounds it was discovered that the sensitivity of the cells, in many but not all instances, depended on the presence of an established glycocalyx on the catenae and spheroids. Accordingly, if test compounds were added immediately or soon after seeding the cells (typically within one day), the cells were sensitive to the compound. However, if compounds were added several days later (typically 3-7 days), the glycocalyx had sufficient time to reestablish, and the cells became increasingly more resistant to the compound. In some cases, that resistance could be several orders of magnitude more than the compounds most sensitive effect on the cells. This effect was reversible if the glycocalyx was removed, thus rendering the cells once again sensitive to the compound. The acquired drug resistance overtime suggests that it is related to the resynthesis and organization of the pericellular glycocalyx. Hence, the glycocalyx may present a selective barrier to compounds depending on their chemical properties (size, polarity, hydrophobicity, diffusion). These observations lead to two further aspects of the present invention, (1) another screening methodology and (2) new methods of treating serosal cancer.

Accordingly, a still further aspect of the invention provides a method to screen a test compound for anti-proliferative or morphological effects which comprises (a) dissociating serosal catenae and preparing a homogenous population of single cells; (b) seeding and culturing those cells for a time and under conditions to produce catenae with an established glycocalyx coat; (c) contacting the cultures with at least one test compound for a time that would be sufficient to allow untreated cultures to proliferate without reaching confluency, i.e., the cultures should remain subconfluent during the course of the screening assay); and (d) determining whether the test compound inhibits proliferation of the catenae or alters morphology of the catenae in the treated culture. In a preferred embodiment, the test compound(s) is added to the culture on day three, four, five, six or seven day post seeding, and more preferably on day five or six. In a variation on this method, following step (b) but prior to step (c), the culture can be incubated for a time and with an amount of a hyaluronidase, a collagenase or both, sufficient to remove or disrupt the glycocalyx coat of said catenae. Such treatments are typically done for about 5-30 minutes at 37° C., and preferably for about 10 minutes. These enzymes do not need to be removed for the duration of the remainder of the assay. Modified and PEGylated versions of the enzymes can also be used in the methods of the invention. These assays can also be readily adapted to an HTS format as above. To determine whether a test compound(s) affects proliferation the cells can be counted manually with or without staining or a fluorescent signal, a luminescent signal or absorbance measured. Because the catenae exist in suspension, detection methods need to be adapted accordingly and can be done by those of skill in the art. One preferred detection method is using alamarBlue® staining, followed by measuring fluorescence or absorbance of the culture which is proportional to the live cells present in the culture and is independent of whether the cells are adherent or in suspension.

A similar assay system for serosal spheroids is also provided. For spheroids, the dissociated cells are cultured for a time and under conditions to produce spheroids of sufficient number and size with an established glycocalyx coat. Because spheroids are large aggregates of many cells, it takes longer to reestablish the coat than it does for catenae. The time frame for spheroids is typically from about 8 to about 14 days, so that adding test compounds is done in that time frame, and preferably at 11 days post seeding.

Yet another aspect of the invention is directed to a method to treat serosal cancer in a patient undergoing chemotherapy or radiation treatment which comprises administering a hyaluronan synthase inhibitor, a hyaluronidase, a collagenase, or other enzyme or other agent that removes or degrades the glycocalyx for a time and in an amount to augment said regimen or treatment, or to improve or increase patient survival time, or to cause remission of symptoms. Such methods include co-administering radiation treatment or chemotherapy and a hyaluronan synthase inhibitor or an enzyme or other agent that removes or degrades the glycocalyx. These enzymes and agents can be PEGylated or otherwise modified to increase their in vivo half life.

Another embodiment is directed to a method to inhibit cancer stem cell self-renewal or formation in a patient which comprises administering an inhibitor of glycocalyx formation or a agent that degrades glycocalyx for a time and in an amount to said patient and thereby inhibit self-renewal or formation of CSC or cause differentiation of CSC and make them susceptible to killing. Such a method can prevent catenae from undergoing spheroid formation, which in turn prevents the CSC from acquiring resistance to standard cancer treatment regimens.

Another aspect of the invention relates to the discovery of HAS2 splice variants and mutant forms of HAS2 in catena and in patient samples. Accordingly, this invention provides isolated nucleic acid encoding a mammalian HAS2 splice variant, including mRNA and cDNA therefore as well as nucleic acids comprising a contiguous nucleotide sequence, in 5′ to 3′ order, that consists essentially of the entirety of or a portion of exon 2 and the entirety of exon 3 of a HAS2 gene, i.e, splice variants that lack exon 1. One mRNA HAS2 splice variant encodes a protein that begins at amino acid 215 of the wt human HAS2 and ends at the normal stop signal, i.e., amino acid 552. The invention also includes vectors comprising any of the nucleic acid of the invention, cells comprising these vectors, as well as using recombinant expression systems produce the encoded proteins, and the encoded proteins. Other embodiments of the invention are directed to isolated nucleic acid probes that are for specific for detecting a mammalian HAS2 splice variant RNA or any one or more HAS2 mutations, including SNP mutations, and preferably detect the mutations identified in Tables 17 and 18. The invention thus also includes mutant and allelic forms of the wt HAS2 and HAS2 splice variants.

Yet another aspect of the invention is drawn to a method of monitoring and/or staging serosal cancer in a subject which comprises (a) preparing catenae from ascites obtained from a cancer patient; (b) detecting whether the catenae have one or more HAS2 mutations and/or express one or more HAS2 splice variants; and (c) correlating those mutations and/or variants with the presence and/or progression of cancer in a said patient. Further, one can identify or monitor for the presence of serosal cancer stem cells in a patient sample by (a) obtaining a cellular sample from a patient; (b) optionally, depleting that sample of leukocytes; (c) preparing DNA, RNA or both from the remainder of the sample; and (d) detecting whether the DNA, RNA or both has a HAS2 mutation or expresses a HAS2 splice variant, with the identification of a mutation or a splice variant indicating the presence of serosal cancer stem cells in the sample. By quantitating the amounts of such DNA or RNA, one can correlate the findings with the presence of serosal cancer and/or progression of a serosal cancer in the patient.

The extensive characterization of the catenae has lead to the discovery of multiple ways to identify catenae, including by identification of specific surface antigens, catena gene signatures, surfaceome-related catena gene signatures, surfaceome-related catena protein signatures, miRNA-related catena signatures, catena cluster-defining gene signatures, exosomal catena protein signatures, secretome catena protein signatures, glycocalyx signatures, activated phosphoprotein expression, and identification of a low molecular weight complex of hyaluronan and collagen that binds to an anti-COL1A2 antibody. These properties have lead to a variety of methods to identify and/or monitor for the presence of serosal cancer stem cells in a patient sample and provides the ability to for personalized medicine approaches to serosal cancer therapy, including the ability to alter a therapeutic regimen in response to the presents of serosal cancer stem cells.

These methods can be performed with serosal fluid, ascites, blood or tumor tissue from a mammal and using a variety of detection techniques including without limitation detecting the nucleic acids in these assays or determining expression levels thereof by microarray analysis, by an RNA or DNA sequencing technique, by RT-PCR or by Q-RT-PCR. Protein detection methods include but are not limited to mass spectrometry, Western blotting, antibody binding with FACS and other techniques with in the ken of the skilled artisan or later developed techniques.

Further, from identifying and/or monitoring serosal cancer stem cells, this information allows development of additional methods of the invention including, a method to detect serosal cancer, to monitor efficacy of a cancer therapy regimen, to categorize patients for therapy, to monitor drug efficacy, to predict a patient response to a cancer therapy regimen in a serosal cancer patient which comprises periodically performing one or more of these methods with samples from a patient and correlating the results with the status of the patient and thereby detect serosal cancer, monitor efficacy of a cancer therapy regimen, categorize a patient for therapy, monitor drug efficacy or predict a patient response to a cancer therapy regimen. Similarly, the invention relates to a method to treat a serosal cancer which comprises (a) administering an anticancer regimen to a serosal cancer patient; (b) periodically reviewing the results from one or more of these methods performed with samples from said patient, and (c) altering the treatment regimen as needed and as consistent or predicted by the results.

Still another aspect of the invention is directed to a method to screen for a metastatic inhibitor or a metastatic effector using in vivo animal models. This method comprises (a) intravenously injecting an immunocompromised, non-human mammal with a preparation of catenae or catena cells, (b) administering one or more test compounds to the mammal before, after or simultaneous with injecting, and (c) assessing the time course of tumor production and/or tumor location in the mammal relative to that of a control mammal and to thereby identify compounds which inhibit metastasis of catena cells, particular as those compounds which reduce or inhibit tumor production or changes in tumor locations.

A still further aspect of the inventions, provides another in vivo method using an animal model to screen for drug efficacy. This method comprises (a) intraperitoneally injecting an immunocompromised, non-human mammal with a preparation of catenae or catena cells; (b) administering one or more test compounds to the mammal before, after or simultaneous with injecting; and (c) assessing (i) the time course of tumor production in said mammal, (ii) the time course of serosal fluid production in said mammal, (iii) the morphology of tumors in said mammal, (iv) the quantity of and/or time course of production of serosal cancer stem cells in the ascites of said mammal, or any combination thereof relative to that of a control mammal and to thereby determine the potential or actual efficacy of a drug compound in treating serosal cancer.

In another aspect, the present invention is drawn to a method to produce spheroids from primary serosal tumor-derived catenae or from metastatic tumor cells which comprises culturing a suspension of catenae or cells for a time in a first serum-containing media supplemented with an amount of Matrigel sufficient to induce spheroid formation and to produce a spheroid culture system. These cultures are periodically supplementing with serum-containing media without additional Matrigel, typically on a weekly basis. Preferably, the ratio of first serum-containing media to Matrigel is 50:1.

A method to produce catenae from serosal fluid of a patient is yet another aspect of the invention. In this method, one obtains a sample of serosal fluid from a cancer patient, harvests the cells from the fluid and cultures those cells in serum-containing media supplemented with cell-free serosal fluid. The cells in the suspension culture are periodically passaged into fresh serum-containing media supplemented with cell-free serosal fluid to thereby obtain catenae. In a preferred embodiment, the serosal fluid is from the same cancer patient and is supplemented at a ratio of 1:1 with media.

The instant invention also provides PCR primer sets comprising PCR primers for mammalian genes identified by the extensive characterization of the catenae. Another aspect of the invention provides a method to prepare catena cells and spheroids, or any cell with a glycocalyx coat, for electron microscopy.

Finally, in any of the foregoing methods or products, as applicable, serosal can be ovarian. Likewise those methods, cells, nucleic acids, vectors, proteins or genes indicated as mammalian include or can be human, murine, porcine, bovine or ovine mammals as applicable.

### BRIEF DESCRIPTION OF THE DRAWINGS

FIG. 1 illustrates an orthotopic ovarian cancer model with NSG mice. NSG mice were injected i.p. with 50,000 Ovcar3-GTL cells. Mice were injected three times a week i.p. with either PBS (phosphate-buffered saline) or with 36 mg/kg of a lipidated oligonucleotide (oligo) for 12 weeks. Tumor growth in PBS-treated group (▴) reached an equilibrium after 12 weeks. Oligo-treated mice (♦) had continuous tumor growth.

FIG. 2 depicts bioluminescent images showing the effect of thioglycollate on intraperitoneal tumor growth. NSG mice were injected i.p. with 10⁶ Ovcar3-GTL cells. Four weeks later, mice were injected i.p. with PBS or 1 mL of thioglycollate solution. Images were obtained at 8 weeks.

FIG. 3 shows photographs of the cell fractions from the ascites of NSG mice injected i.p with 50,000 Ovcar3-GTL cells and harvested at 8 weeks after treatment with a lipidated oligonucleotide. The ascites was passed through a 40 μm filter. (a) The >40 μm fraction contains large, preformed spheres; (b) the flow-through fraction contains smaller, preformed chains of cells (catenae); and (c) ficoll fractionation removes RBCs from the catenae fraction. In (b), a glycocalyx visibly separates the catenae from the RBCs in the ascites.

FIG. 4 is a schematic representation of an in vitro culture system for enrichment of catenae. (a) Ovcar3-GTL tumor cells from ascites are cultured in 10% FCS on tissue culture treated plates; (b) and (c) suspension fractions are re-passaged weekly; (d) after continuous passages, cultures are enriched for free-floating catenae.

FIG. 5 show immunofluorescence staining of a catena for (a) tight junction protein ZO-1 and E-cadherin, and (b) for giantin (a golgi marker) and human vimentin. Panel (c) is a photograph of a non-attached catena developing in culture. In (a), the bright punctuate staining at the cell junctions is from ZO-1. In (b), the bright globular staining is from giantin and the light grey staining is from vimentin.

FIG. 6 shows photographs of Ovcar3-GTL-derived catenae cultures with sphere formation. Ovcar3-GTL catenae formed spheroids by rolling-up (arrowed) at high cell density.

FIG. 7 present photographs of and a schematic representation of spheroid and catenae formation. Ovcar3-GTL sphere-forming cells (red) pile up on mesenchymal monolayers (white) [stage 1-2], and form organized spheroids by budding [stage 3]. Catenae (blue) are observed inside [stage 4] or migrating out of developing spheroids [stage 5]. Developed spheroids detach from monolayers and continue to grow in suspension [stage 6] where more catenae are extruded into suspension.

FIG. 8 graphically depicts the percentage clonogenicity from in vitro clonogenic assays with Ovcar3-GTL catenae, spheroids and monolayers (left, center and right bars, respectively). For the first clonogenic assay, catena/spheroid mixed cultures were separated into >40 um (spheroid) and <40 um (catena and small spheroids) fractions. The number of clones was scored at week 2. After the third single cell recloning passage, catena had 55% clonogenicity, spheroids had 10% and monolayers had 1%.

FIG. 9 graphically illustrates the results of a tumor-initiating, limiting-dilution assay in immunodeficient mice used to assess CSCs in catenae and spheroids. The left panel displays the bioluminescence from NOD-SCID (solid bars) and NSG (open bars) from mice injected i.p. with the same number of dissociated Ovcar3-GTL monolayer cells, dissociated Ovcar3-GTL catena cells and undissociated Ovcar3 spheroids (spheres). The right panel displays the bioluminescence from NSG mice injected i.p. with varying number of the same monolayer and catena cells.

FIG. 10 depicts bioluminescent images from subcutaneous limiting dilution experiments in NSG mice injected with 200, 20 and 2 Ovcar3-derived catena cells in Matrigel™. Images were taken at week 3 after injection.

FIG. 11 shows that mesenchymal cells grown in suspension culture can generate catenae and spheroids. The top panel shows typical cultured monolayers of Ovcar3 (epithelial cells), Ovcar5 (mesenchymal cells) and A2780 (mesenchymal) cells. The middle panel shows Ovcar5 cells from suspension cultures with (a) clumping up on monolayer cells, (b) spheroids with cystic structures, (c) catenae in suspension, and (d) a sphere extruding catena. The bottom panel shows cells from A2780-G suspension cultures with (e) a collective amoeboid transition and (f) catenae.

FIG. 12 graphically illustrates a model of the catena-spheroid concept.

FIG. 13 is a bar graph showing the amount of CA125 (MUC16) secreted into the culture medium by subconfluent Ovcar3-GTL epithelial monolayers and catena as measured by ELISA.

FIG. 14 displays photographs of a particle exclusion assay using RBCs for (a) mechanically dissociated Ovcar3-GTL catenae and (b) hyaluronidase treated Ovcar3-GTL catenae.

FIG. 15 is a series of scanning electron microscope (SEM) images of a catena showing the glycocalyx. Alcian blue (AB) is used to visualize the hyaluronan sugar chains and cetylpyridinium chloride (CPC) is used to visualize the proteoglycans. The catena and glycocalyx are shown in (a) with a bar representing 10 μm. In (b) the same image is magnified 2×, in (c) the same image is magnified 5× and in (d) the same image is magnified 10× (all relative to the image in (a). The arrow points to a single cell in the catena.

FIG. 16 is an enlarged SEM image of the catena and glycocalyx stained only with Alcian blue, showing the hyaluronan coat over the cells and the web like nature of the glycocalyx. Hyaluronic acid concentrates at various points.

FIG. 17 is an SEM image of a catena after treatment with hyaluronidase to remove the glycocalyx coat. Staining was done with Alcian blue and CPC.

FIG. 18 is an SEM image of a catena after treatment with hyaluronidase to remove the glycocalyx coat. The other cells present in the sample are RBCs. The image was obtained without staining.

FIG. 19 shows SEM micrographs (a,b,c) without staining of catena cells. (a) SEM of catena showing an area of attachment between two cells with extensive microvilli connections. (b) Two catena cells connected by a nanotube. Note microvilli attaching the cells to the surface (invadopodia). Cells are characterized by microvilli and large plasma membrane blebs. (c) SEM of catena cells with a long (20-30 um) pseudopodium extending beyond the 10-15 um hyaluronan glycocalyx.

FIG. 20 is an approximate 3-fold enlargement of the photograph in FIG. 19(a) with the tailed, white arrow pointing at microvilli, the black arrow pointing to pseudopodia and tailless white arrow point to surface blebs.

FIG. 21 is an SEM showing a side view of (a) an erupting “volcano” on the catena surface and (b) an enlargement of the volcano showing the release of particles from the crater of the volcano.

FIG. 22 provides a table showing the differential regulation of hyaluronan synthesis pathway in Ovcar3 epithelial monolayers, Ovcar5 mesenchymal monolayers and in Ovcar3 and Ovcar5 catena as determined by microarray analysis. Down regulated genes are in grey; up regulated genes are in black. The values in the catena column (*) represent mRNA copies number determined by 454 deep sequencing.

FIG. 23 is a dot blot showing the RTK phosphorylation pattern in epithelial (Ovcar3 monolayers), mesenchymal (Ovcar5 monolayers) and catena cells (Ovcar3 and Ovcar5) as determined with a Human Phospho-RTK Array Kit.

FIG. 24 depicts differential expression of selected CD proteins for Ovcar3 catena (CSC 65%) and Ovcar3 epithelial monolayers (CSC 1%).

FIG. 25 illustrates the genomic structure of a wild type (wt) HAS2 gene, showing the intron and exon structure and indicating the nucleotides defining each element (top). The bottom panel illustrates the mRNA structure of the HAS2 splice known as the Greenwich variant which contains an in-frame deletion of exon 1 and a portion of exon 2.

### DETAILED DESCRIPTION OF THE INVENTION

1. Overview

The present invention provides a clonally pure population of serosal cancer stem cells (CSCs), and methods of preparing and culturing these CSCs. With the availability of pure CSCs, extensive characterization of the cells is possible and has lead to the elucidation of cell markers, morphology of the cells, identification of specifically expressed genes, identification of surfaceome markers, secretome markers, and from this information, target pathways for development of therapeutics and new treatment regimens. Purified CSCs are obtained as free-floating chains of cells, which are termed herein as catenae (plural; catena in the singular), with the capacity to self-renew and to differentiate. In addition to the serosal catenae, the invention provides purified serosal spheroids and methods of isolating these cellular entities, allowing similar characterization studies of the spheroids at the molecular level.

The serosal cavity is a closed body cavity that includes and encloses the peritoneal, pleural, and pericardial cavities of the body, is fluid filled (serosal fluid) and is bounded by the serous membrane. Serosal cancers include the primary cancers that arise within the serosal cavity and secondary cancers that arise by metastasis of other cancer cells into the serosal cavity. Major serosal cancers at different serosal sites include those in (1) pleural effusions, namely mesothelioma, bronchogenic lung cancer, breast cancer, bladder cancer, ovarian cancer, fallopian tube cancer, cervical cancer and sarcoma; (2) peritoneal effusions, namely ovarian cancer, fallopian tube cancer, gastric cancer, pancreatic cancer, colon cancer, renal cancer and bladder cancer; and (3) pericardial effusions, namely mesothelioma, bronchogenic lung cancer, breast cancer, bladder cancer, ovarian cancer, fallopian tube cancer, cervical cancer and sarcoma. The list is not exhaustive, and any other cancer that metastasizes to any serosal cavity and forms tumors can be considered as a “serosal cancer.”

2. Miscellaneous Definitions

Serosal cells are any cells originating from or found within the serosal cavity or forming or attaching to the serous membrane, and include, but are not limited to, ovarian, endothelial, stomach, intestinal, anal, pancreatic, liver, lung and heart cells.

As used herein, NSG and NSG mice mean the NOD scid gamma (NSG) mice, or an equivalent, available from The Jackson Laboratory and which are the NOD.Cg-Prkdc^{scid} Il2re^{tm1Wj1}/SzJ JAX® Mice strain. The NOG strain of mice are similar to NSG mice but have a truncated IL-2 receptor gamma chain rather than a complete null allele of the NSG mice.

As used herein, “chemotherapy” includes any form of cancer therapy in which one or more drugs is administered to a cancer patient for any and all cancer-related purposes, including without limitation, cytotoxic agents that inhibit or kill tumor cells (or other malignant cells) and cancer stem cells as well as agents that act in a cytostatic manner on such cells. Such drugs include, but are not limited to, small molecules, antibodies, proteins, nucleic acids, target pathway inhibitors and the like. For the avoidance of doubt, chemotherapy, as used herein, also includes pathway inhibitor therapy such as occurs when a subject has a genetic mutation in a specific gene and is administered a therapeutic agent targeted at that gene or the metabolic or regulatory pathway of which that gene forms part.

The abbreviations “ip” and “i.p.” are used interchangeably for intraperitoneal or intraperitoneally.

As used herein, ‘PEGylated” refers to a polyethylene glycol moiety (PEG) attached to a protein or other molecule of interest. PEGylation refers to the process of attaching a PEG to a protein or other molecule. Methodology for such modification is known in the art.

3. Catenae

Clonally pure serosal CSCs are self-renewing serosal cells capable of differentiation and by this criterion meet the definition of stem cells. The CSCs comprise free-floating chains of cells having anywhere from three to four cells per chain to about seventy-two (72) cells, but this is not a precise upper bound as longer catena are occasionally observed. The catenae are surrounded by a glycocalyx comprising hyaluronan and resist attachment to tissue culture plates. As described in the methods of the present invention, catenae can be propagated in suspension cultures indefinitely. Each catena is clonal and cell division takes place symmetrically along the same axis, with occasional branching being observed. The capacity for symmetric division is independent of a cell's position in the chain, meaning that cells at the end and the middle of divide symmetrically and independently along the chain axis. This capacity to divide and propagate in culture establishes that the catena cells are self-renewing.

The cells are attached to each other via tight junctions which stain positively for ZO-1 but are negative for the presence of E-cadherin. Time lapse photography has shown that catenae do not fuse with each other but appear to repel each other.

When assessed in vitro, the catenae show at least 50% serial recloning capacity in limiting dilution assays. The individual catena cells have substantially increased in vivo engraftment potential relative to serosal epithelial tumor cells. Under appropriate conditions one or two catena cells can lead to engraftment of a tumor in a mouse cancer model. For example, in vivo engraftment is 50-100% in certain mice models (NSG mice) implanted subcutaneously with single catena cells in Matrigel. The catena engraft greater than 10,000 fold better over epithelial monolayers. This ability to form tumors after in vivo transplantation establishes that catenae have differentiation potential. Moreover, the tumors formed have similar morphology to those from which the cells were originally derived.

Similarly, catenae have the capacity to generate epithelial and mesenchymal monolayers in vitro under the appropriate conditions. It has been discovered that removing the glycocalyx (e.g., by hyaluronidase treatment) causes catenae to stop growing in suspension culture, settle onto tissue culture plates and begin to differentiate into mixed cultures of epithelial and mesenchymal cells.

Catenae grown in culture will continue to produce catenae, i.e., catenae are capable of serial passage in culture as non-attached cells. However, under appropriate conditions, such as when cultures become saturated, the catenae can round up and form spheroids. This rolling up action may provide a physical barrier means to protect CSCs from adverse conditions as spheroids contain about 10-30% CSC.

Catenae can be produced from serosal epithelial cancer cells or serosal mesenchymal cancer cells (discussed in detail below). Epithelial cells have polarized morphology and are E-cadherin positive and vimentin negative. Mesenchymal cells show a spindle morphology and are E-cadherin negative and vimentin positive. Catenae cells are rounded, and like mesenchymal cells, are E-cadherin negative and vimentin positive.

The catena's glycocalyx coat of hyaluronan is a predominant morphological feature and can be removed by treatment with hyaluronidase. The glycocalyx extends up to approximately 20 μm around the catena cells. When the glycocalyx is present, catenae grow in suspension culture and do not interact with extracellular matrix component. When the glycocalyx is removed enzymatically, the catena cells attached to surfaces, and form filopodial extensions and exhibit multilineage differentiation potential. Mechanically-dissociated catena cells remain in suspension and proliferate rapidly to form free-floating chain.

Scanning electron microscopy (SEM) of catena cells have shown a variety of pericellular structures in addition to the glycoclayx, including microvilli, nanotubes, pseudopodia, antenna and filopodia. In some instances, microvilli have been observed all over the cells and in other instances they tend to be located at the cell junctions, suggesting a role in cell-to-cell adhesion. The nanotubes are a novel cellular feature of CSCs and appear involved in cell-to cell communication, possible allowing passage of biomolecules between cells. The pseudopodia, antenna and filopodial may play a role in formation of the nanotubes as well as allow surveillance of the environment for attachment surfaces and the presence of cytokines, growth factors and immune cells.

In addition, SEM has shown that the catena cells have surface blebs and structures that appear to erupt from the cell surface and release smaller particles. These erupting structures appear as either “volcanoes” or invaginated “craters.” The released particles are similar in appearance and size to the surface blebs and appear to be exosomes.

Transmission electron microscopy (TEM) shows that the catena cells have the undifferentiated cell morphology (high nucleus to cytoplasm ratio) typical of stem cells. TEM also allowed observation of the tight junctions between the cells and showed that intact functional mitochondria are present. Surface blebs were observed to be contiguous with the cell membrane and to contain ribosomes.

Having a clonally pure population of cells allowed molecular characterization of ovarian catenae (i.e., ovarian CSCs). Using gene expression, the invention provides the gene signature for ovarian catena relative to ovarian mesenchymal monolayer cancer cells shown in Table 5. The gene signature has 26 upregulated genes and 69 down regulated genes, with hyaluronan synthase (HAS2) the most highly expressed gene in catenae/CSCs. The second most expressed gene was PDGFRA indicating a significant role for the PDGF pathway in catenae/CSCs.

Using differential miRNA expression analysis, it was discovered that the miR-200 family (miR-141, miR-200a, miR-200b, miR-200c and miR-429) and the Let-7 family miRNAs were significantly down-regulated in the ovarian catenae compared to ovarian epithelial monolayers. Further, hsa-miR-23b and hsa-miR-27b were significantly down regulated in ovarian catena compared to ovarian mesenchymal monolayers.

Using a receptor tyrosine kinase (RTK) phosphorylation assay, it was shown that ovarian catenae cells and ovarian mesenchymal cancer cells have qualitatively similar phospho-RTK profiles.

Using cell surface marker analysis with commercially available antibodies and FACS, ovarian catenae are positive for the markers CD49f (α6-integrin), CD90, GM2 and CD166 and negative for the markers EpCam (CD326), Muc16(CA125) and CD44.

4. Spheroids

Serosal spheroids are large cellular structures composed of tens of thousands of cells were observed as entities that would not pass through a 40 μm filter. Spheroids may play a role in metastasis and tumor formation. Spheroids also self-renew in suspension cultures and have differentiation capacity. When assessed in vitro, spheroids have about a 10% serial recloning capacity in limiting dilution assays.

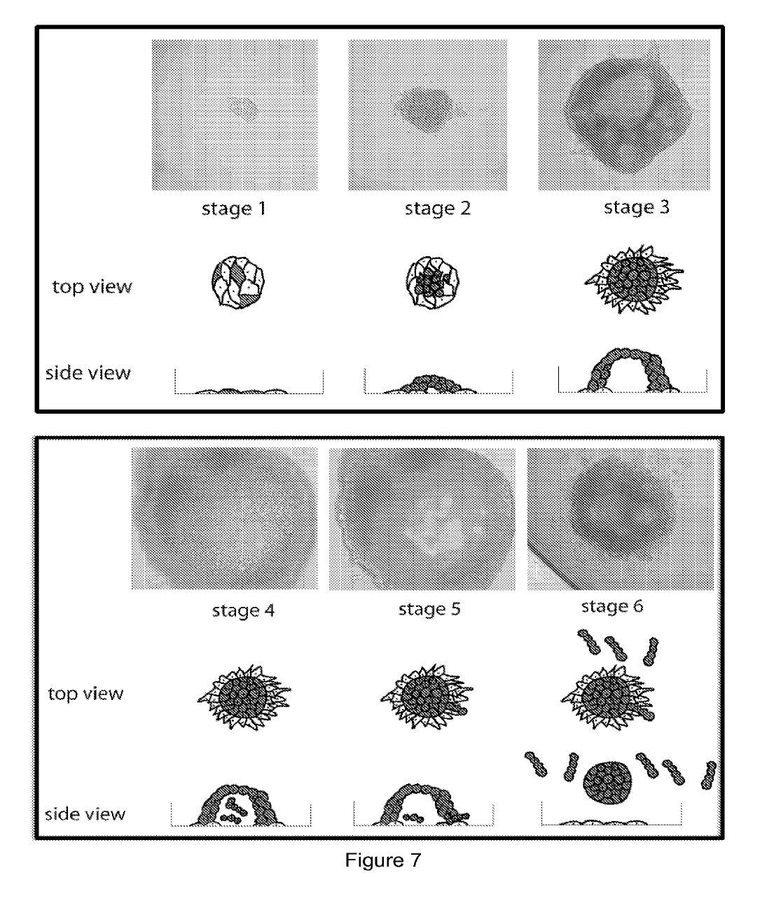

Spheroids developed from catenae by a process of “rolling up,” suggesting that during nutrient deprivation at confluent stages of cell culture, spheroids provide a protective environment for catenae survival. Additionally, cells can amass on attached mesenchymal monolayers and begin to form spheroids. This cell mass grows in the vertical direction relative to attachment surface, resembling “budding” from attached cells, and develops into spheroids with organized cystic structures. The spheroids eventually detach from attached monolayers and continue to rapidly proliferate in suspension while maintaining the sphere morphology. A schematic diagram of this process is shown in FIG. 7. Developing spheroids extrude fresh catenae into the suspension which in turn can proliferate rapidly to form new floating catenae.

5. Preparation of Catenae and Spheroids

The present invention relates to methods of preparing catenae and spheroids. Two principal methods are described herein. In one method, serosal epithelial or mesenchymal cancer cells are injected intraperitoneal (ip) into an animal tumor model (preferably mice), preferably with the addition of an inflammatory stimulus. After sufficient time to develop ascites and/or solid tumors, the ascites is harvested from ip tumor-bearing animals and separated into two or more size fractions, preferably two fractions. The smaller size fraction contains the catenae and single cells, typically leukocytes. The leukocytes can be readily removed and the remaining cells serially passaged in suspension culture to obtain a self- renewing population of clonal serosal catenae. The larger fraction includes the spheroids retained on the filter. These spheroids are collected and serially passaged in a suspension culture to obtain a self-renewing population of spheroids.

The source of the serosal epithelial cells can be from primary serosal cancer cells, or immortalized epithelial or mesenchymal serosal cancer cell lines. The primary cancer cells or cell lines can be from primary cancers or metastatic tumors. Preferably the serosal cancer cells are ovarian cancer cells.

As used herein, an animal tumor model is an animal capable of allowing tumor formation and is typically highly immunodeficient, i.e., lacking at least B cells and T cells and preferably also NK cells. For example, a preferred animal is a NOD-SCID ILR gamma (−/−) mouse (referred to herein as a “NSG” mouse) which lack B cells, T cells and NK cells. NOD-SCID mice lack B cells and T cells, and while useful, require injection of much greater more cell numbers to develop tumors.

Inflammatory stimuli include any agent, drug or factor (collectively referred to herein as inflammatory agents) that stimulate inflammation in an animal, and are preferably administered i.p. Inflammatory agents include, but are not limited to, lipidated oligonucleotides, thioglycollate; chemerin; macrophage migration-inducing chemokines such as chemokine (C-C motif) ligand 1 (CCL1), CCL2, CCL4, CCL7, CCL8, CCL12, CCL13, CCL15, CCL16, CCL23 and CCL25; macrophage activating chemokines such as CCL14; and various agents of bacterial origin including, brewer's thioglycollate broth (3%.), BCG heat-killed (cell walls from M. bovis), pyran copolymer, C. parvum heat-killed whole cells, pyridine extract of C. parvum, detoxified endotoxin from Salmonella typhimurium; and sodium metaperiodate. The lipidated oligonucleotides are typically small oligomers of from about 8 to about 30 nucleotides and act in a sequence independent manner. The lipid moiety can be any convenient group such as myristate, palmitate and the like. Those of skill in the art can determine appropriate doses for administering inflammatory agents.

Size fractionation can be done by passing the ascites through one or more filters. Useful filter sizes range from about 20-60 μm, with larger sizes allowing more spheroids to pass through. A preferred filter size is 40 μm.

In another method, catenae and spheroids can be produced by in vitro culture techniques from immortalized serosal mesenchymal cancer cells. In this method, the mesenchymal cells are grown as monolayers, the culture supernatant is harvested and the suspension cells are pelleted by gentle centrifugation (e.g., at 300 g for 1-5 minutes). The pelleted cells are resuspended in fresh media (typically at one-tenth the previous culture density), transferred to fresh suspension culture flasks for growth. Repeating this cycle several times produces self-renewing populations of serosal catenae and spheroids. Typically the cells are grown until they reach a cell density of about 200,000 cells/mL or can be passaged weekly. Likewise, this process appears to remove an inhibitory factor produced by mesenchymal monolayers that prevents catenae and spheroid formation. These cultures can be size fractionated as above to separate the catenae from the spheroids.

The growth media for these methods is any convenient media supplemented with 10% fetal calf serum (FCS). Cells are generally grown at 37° C. with 5% CO₂. A preferred growth media for catenae is M5 with 10% FCS (Hyclone) and 1% P/S (Pen-Strep Solution at 10,000U/mL penicillin G and 10 mg/mL streptomycin; Gemini Bio-Products), designated hereafter as M5-FCS. M5 media is DME:F12, 6 g/L HEPES and 2.2 g/L sodium bicarbonate. Catenae can also be grown in serum-free, protein-free media supplemented with insulin. One such preferred media is M5 with 1% P/S and 0.1 U/mL recombinant insulin. The insulin source should be the same as the cell source, i.e., if human catenae are being cultured, the serum free media is supplemented with recombinant human insulin, etc.

A preferred growth media for spheroids is ES media, and preferably supplemented mTeSR1 media [Ludwig et al. 2006].

6. Gene Signature and Other Methods to Identify CSCs

The gene expression information provided in Table 5 may be used as diagnostic markers for the identification of the ovarian CSCs. For example, ascites or an ovarian tissue sample from a patient may be assayed using a gene microarray, RNA sequencing, RT-PCR, Q-RT-PCR, 454 deep sequencing, or other methods known to those of skill in the art, to determine the expression levels of one or more of the genes in Table 5. These levels may be compared to the expression levels found in normal tissue, ovarian mesenchymal cancer cells or ovarian epithelial cancer cells. Expression levels can also be used as markers for the monitoring of disease state, disease progression, especially metastasis, or as markers to evaluate the effects of a candidate drug or agent on a cell or in a patient. Assays which monitor the expression of a particular genetic marker or markers can utilize any available means of monitoring for changes in the expression level of the relevant genes. As used herein, an agent is said to modulate the expression of a gene if it is capable of up- or down-regulating expression of mRNA levels of that gene in a cell.

The present invention provides the following methods to identify and/or monitor for the presence of serosal cancer stem cells in a patient sample.

With respect to the catena surfaceome, is provided a method to identify and/or monitor for the presence of serosal cancer stem cells in a patient sample which comprises (a) obtaining a cellular sample from a patient; (b) depleting the sample of leukocytes; (c) reacting the sample with a panel of detectable surface antigen antibodies; (d) sorting the reacted cells into single- or multi-cell samples; and (e) detecting whether any of said single- or multi-cell samples are positive for the presence of CD49f, CD90, CD166, PDGFRA, and GM2 proteins and negative for the presence of CD34, CD133, MUC16 and EPCAM proteins, wherein the presence and absence of said proteins identifies the reacted cells as containing serosal cancer stem cells or identifies a single cell as a serosal cancer stem cell.

Sorting cells, including to the single cell level, can be done, for example, by fluorescent activated cell sorting (FACS) using appropriately distinguishably labeled antibodies.

Alternatively, surfacesome characteristics can be used in a method to identify and/or monitor for the presence of serosal cancer stem cells in a patient sample which comprises (a) obtaining a cellular sample from a patient; (b) depleting the sample of leukocytes; (c) extracting RNA from the remainder of the sample; (d) analyzing the RNA for expression levels of a human mRNA transcriptome; and (e) identifying samples having a surfaceome-related catena gene signature as those which have upregulated HAS2 and PDGFRA, downregulated MUC16 and EPCAM and have upregulated at least 7 additional genes listed in Table 11, wherein having those characteristics indicates the patient sample contains serosal cancer stem cells.

Likewise, the surfaceome properties can be used in a method to identify and/or monitor for the presence of serosal cancer stem cells in a patient sample which comprises (a) obtaining an integral membrane protein fraction from a cellular sample of a patient, wherein the cellular sample has optionally been depleted of leukocytes; (b) analyzing the protein content of said membrane fraction by mass spectrometry; (c) identifying samples having a surfaceome-related catena protein signature as those samples in which the spectral data indicate the presence of at least 40 proteins listed in Table 16, wherein presence of those proteins indicates the patient sample contains serosal cancer stem cells. One method to prepare an integral membrane fraction is to isolate cells and use phase partitioning process with Triton X-114 to prepare a detergent soluble fraction that can be analyzed by mass spectrometry.

Based on the information from the catena miRNAs that have been characterized, the present invention provides a method to identify and/or monitor for the presence of serosal cancer stem cells in a patient sample which comprises (a) obtaining a cellular sample from a patient; (b) depleting the sample of leukocytes; (c) extracting RNA from the remainder of the sample; (d) analyzing the RNA for expression levels of human miRNA; and (e) identifying samples having an miRNA-related catena signature as those which have downregulated let-7 and 200 families of miRNA, downregulated hsa-miR-23b and hsa-miR-27b, and have upregulated at least 4 additional miRNA listed in Table 8, wherein having those characteristics indicates the patient sample contains serosal cancer stem cells.

Using analysis for the expression of all catena mRNA established a catena gen signature. Hence, another embodiment of the present invention is also directed a method to identify and/or monitor for the presence of serosal cancer stem cells in a patient sample which comprises (a) obtaining a cellular sample from a patient; (b) depleting the sample of leukocytes; (c) extracting RNA from the remainder of the sample; (d) analyzing the RNA for expression levels of a human mRNA transcriptome; and (e) identifying samples having a catena gene signature as those samples which have upregulated HAS2 and PDGFRA and have upregulated at least 5 additional genes listed in Table 5, wherein having those characteristics indicates the patient sample contains serosal cancer stem cells. Another embodiment uses a catena cluster-defining gene signature and provides a method to identify and/or monitor for the presence of serosal cancer stem cells in a patient sample which comprises (a) obtaining a cellular sample from a patient; (b) optionally, depleting the sample of leukocytes; (c) extracting RNA from the remainder of the sample; (d) analyzing the RNA for expression levels of a human mRNA transcriptome; and (e) identifying samples having a catena cluster-defining gene signature as those samples which have upregulated at least six of the nine genes in LIST1 of Table 7 and have upregulated at least 5 of the genes in LIST2 of Table 7, wherein having a catena cluster-defining gene signature indicates the patient sample contains serosal cancer stem cells.

In a related method of the invention, one can identify serosal cancer stem cells in a subject by the method which comprises (a) detecting the level of expression of ten or more genes from Table 5 in a tissue sample, wherein increased or decreased expression of the genes in accordance with Table 5 and relative to expression in serosal mesenchymal monolayer cells is indicative of the presence of serosal cancer stem cells.

The catena exosomes and secretomes are particularly useful for methods of identifying and/or monitoring serosal cancer stem cells. For example, in one embodiment, the exosomal catena protein signature can be used in a method to identify and/or monitor for the presence of serosal cancer stem cells in a patient sample which comprises (a) obtaining isolated exosomes from a patient sample; (b) analyzing the protein content of said exosomes by mass spectrometry, by antibody binding or otherwise; (c) identifying samples having an exosomal catena protein signature as those samples in which the spectral data or other data indicate the presence of CD63, COL1A2 and at least 5 additional proteins listed in Table 13, wherein presence of said proteins indicates the patient sample contains serosal cancer stem cells.

In another embodiment, exosomal catena protein signature can be used in a method to identify and/or monitor for the presence of serosal cancer stem cells in a patient sample which comprises (a) obtaining isolated exosomes from a patient sample; (b) reacting said exosomes with one or more antibodies specific for CD63, COL1A2 and at least 5 additional proteins listed in Table 13; and (c) identifying samples having an exosomal catena protein signature as those samples in which are positive for the presence of CD63, COL1A2 and at least 5 additional proteins listed in Table 13, wherein presence of said proteins indicates the patient sample contains serosal cancer stem cells.

In yet another embodiment, the secretome catena protein signature can be used in a method to identify and/or monitor for the presence of serosal cancer stem cells in a patient sample which comprises (a) obtaining a supernatant fraction from a patient sample from which cells, cellular debris and exosomes have been removed; (b) analyzing the protein content of said supernatant fraction by mass spectrometry; (c) identifying samples having a secretome catena protein signature as those samples in which the spectral data indicate the presence of at least 20 proteins listed in Table 15, wherein presence of those proteins indicates the patient sample contains serosal cancer stem cells.

Still another embodiment uses a glycocalyx signature and provides a method to identify and/or monitor for the presence of serosal cancer stem cells in a patient sample which comprises (a) obtaining a supernatant fraction from a patient sample from which cells, cellular debris and exosomes have been removed; (b) analyzing the protein content of said supernatant fraction by mass spectrometry; (c) identifying samples having a glycocalyx signature as those samples in which the spectral data indicate the presence of at least 6 proteins found in glycocalyx as listed in Table 4 and the absensce of ELN, FN1 and at least 2 protein downregulated in catena as listed in Table 4, wherein presence and absence of those proteins indicates the patient sample contains serosal cancer stem cells.

Based on phosphorylation of tyrosine kinase receptors (RTK), another embodiment of the invention is directed to a method to identify and/or monitor for the presence of serosal cancer stem cells in a patient sample which comprises (a) obtaining a cellular sample or a cell lysate from a cellular sample from a patient, wherein said sample has been depleted of leukocytes; (b) incubating said sample or said lysate with a panel of human tyrosine kinase receptor-specific antibodies and a pan-phosphotyrosine antibody; and (c) detecting whether said sample or lysate is positive for activated phosphoproteins selected from the group consisting of PDGFRA and at least 6 of the proteins selected from the group consisting of PDGFRβ, EGFR, ERBB4, FGFR2, FGFR3, Insulin-R, IGF1R, DTK/TYRO3, MER/MERTK, MSPR/RON, Flt-3, c-rRET, ROR1, ROR2, Tie-1, Tie-2, TrkA/NTRK1, VEGFR3, EphA1, EphA3, EphA4, EphA7, EphB2, EphB4, and EphB6, wherein the detection of said activated phosphoproteins identifies the patient sample as containing serosal cancer stem cells.

Based on the composition and characterization of the glycocalyx, one can identify and/or monitor for the presence of serosal cancer stem cells in a patient sample by a method which comprises (a) obtaining a supernatant fraction from a patient sample from which cells and cellular debris have been removed; (b) reacting the sample with an anti-COL1A2 antibody; (c) detecting whether said antibody binds a low molecular weight complex of hyaluronan and collagen of less than 20,000 Daltons, wherein the detecting said complex indicates that said sample contains serosal cancer stem cells

The samples for the methods in this section can be mammalian serosal fluid, ascites, blood or tumor tissue. Preferably, the mammal is a human.

The various steps of detecting, determining, analyzing and the like can be conducted by methods known to those of skill in the art. For example, with the appropriate methods, detecting of a nucleic acid or determining expression levels can be accomplished by microarray analysis, by an RNA or DNA sequencing technique, by RT-PCR, by Q-RT-PCR and the like.

Further, the above methods form the basis of additional embodiments of the instant invention. For example, this invention provides a method to detect serosal cancer, to monitor efficacy of a cancer therapy regimen, to categorize patients for therapy, to monitor drug efficacy, to predict a patient response to a cancer therapy regimen in a serosal cancer patient which comprises (a) periodically performing one or more methods of the above methods (e.g., as set out in original claims 48-67) with samples from a patient and (b) correlating the results with the status of the patient to thereby detect serosal cancer, to monitor efficacy of a cancer therapy regimen, to categorize a patient for therapy, to monitor drug efficacy or to predict a patient response to a cancer therapy regimen.

Another aspect of the invention provides PCR primer sets for identifying serosal CSCs by any one of the myriad of PCR amplification methods known in the art for DNA, RNA or both. Those of skill in the art can select the appropriate sequences to for the PCR primers from the known sequence of the human genome. The PCR primers sets of the invention for mammalian genes are the following combinations (each combination being a PCR primer set for amplification and detection of the indicated genes within that set):

(a) CD49f, CD90, CD166, PDGFRA and GM2 genes;

(b) CD49f, CD90, CD166, PDGFRA, GM2, CD34, CD133, MUC16 and EPCAM genes;

(c) HAS2, PDGFRA and at least 10 of the upregulated genes listed in Table 11;

(d) HAS2, PDGFRA, MUC16, EPCAM and at least 10 of the upregulated genes listed in Table 11;

(e) the genes of at least 40 of the proteins listed in Table 16;

(f) let-7 and 200 miRNA families, hsa-miR-23b and hsa-miR-27b, and at least 4 additional miRNAs listed in Table 8;

(g) HAS2, PDGFRA and at least 5 additional genes listed in Table 5;

(h) the nine genes in LIST1 of Table 7 and at least 5 genes in LIST2 of Table 7;

(i) ten or more genes from Table 5;

(j) CD63, COL1A2 and at least 5 additional genes for the proteins listed in Table 13;

(k) the genes of at least 20 proteins listed in Table 15;

(l) the genes of at least 6 glycocalyx proteins as listed in Table 4;

(m) ELN, FN1, the genes of at least 6 glycocalyx proteins as listed in Table 4, and the genes of at least 2 proteins listed as downregulated in Table 4; and

(n) PDGFRA and the genes for at 6 of the proteins selected from the group consisting of PDGFRβ, EGFR, ERBB4, FGFR2, FGFR3, Insulin-R, IGF1R, DTK/TYRO3, MER/MERTK, MSPR/RON, Flt-3, c-rRET, ROR1, ROR2, Tie-1, Tie-2, TrkA/NTRK1, VEGFR3, EphA1, EphA3, EphA4, EphA7, EphB2, EphB4, and EphB6.

7. Drug Screening Methods

In one embodiment, the methods of the invention include methods to screen a test compound for anti-proliferative effects by (a) culturing dissociated serosal catena or serosal spheroid cells that are detectable by fluorescence or luminescence; (b) contacting said catena or spheroids with a test compound; (c) detecting proliferation of said catena or spheroids by measuring the fluorescence or luminescence produced by the cultures relative to control cultures; and (d) determining if the test compound inhibits proliferation of said catena or spheroids.

Similarly, another method to screen a test compound for anti-proliferative effects on serosal cancer stem cells comprises (a) culturing dissociated serosal catena cells, dissociated serosal spheroid cells and dissociated serosal cancer adherent cells, each of which are detectable by fluorescence or luminescence, in parallel; (b) contacting said cells with said test compound; (c) detecting proliferation of catena, spheroids and adherent cells by measuring the fluorescence or luminescence produced by the cultures relative to control cultures; (d) determining if the test compound differentially inhibits proliferation of the catenae relative to spheroids and monolayers.

In these methods of the invention, cells are conveniently grown in multi-well plates such as 96-well, 384-well or 1536-well plates. The various manipulations to add media, seed the plates, add test compounds and score the results can be done manually or robotically on apparatus designed for this purpose. Similarly, the assay results can be determined manually, or can be adapted to automated or robotic analyzers. For detecting anti-proliferative effects, the fluorescent signal from the cell cultures can be at assessed at discreet time points or monitored continuously as is suitable for the assay.

In another embodiment, the invention provides methods to screen test compounds (or agents) for phenotypic or other effects on serosal catenae, spheroids and monolayers. These methods are conducted in a manner similar to the above assays to assess the anti-proliferative effects of test compounds, except for the detection method. In these embodiments, the detection method depends on the particular property being assessed and being distinctly detectable. For differentiation inhibitors, the detection method can assess whether catena cells fail to differentiate in culture upon exposure to the compound.

In conducting screening assays with test compounds it was discovered that the integrity of the glycocalyx can play an important role is drug sensitivity or resistance of the cells. While some compounds can readily penetrate the glycocalyx, others cannot. For the compounds used in chemotherapy which eventually cease to be efficacious in a patient, the knowledge that a drug or chemotherapeutic has lost effectiveness due to the possible renewed presence means that such drugs could maintain efficacy, and hence be used again, if the glycocalyx of the serosal cancer stem cells could be removed. This recognition created a need for another way to screen test compounds or drugs, know chemotherapeutics and the like for the ability to inhibit proliferation or alter the morphology of catena and spheroids under conditions where these cellular entities have of an established and/or substantial glycocalyx.

Accordingly, another embodiment of the invention provides a method to screen a test compound for anti-proliferative or morphological effects which comprises (a) dissociating serosal catenae and preparing a homogenous population of single cells; (b) seeding and culturing those cells for a time and under conditions to produce catenae with an established glycocalyx coat; (c) contacting the cultures with at least one test compound for a time that would be sufficient to allow untreated cultures to proliferate without reaching confluency, i.e., the cultures should remain subconfluent during the course of the screening assay); and (d) determining whether the test compound inhibits proliferation of the catenae or alters morphology of the catenae in the treated culture. In a preferred embodiment, the test compound(s) is added to the culture on day three, four, five, six or seven day post seeding, and more preferably on day five or six. In a variation on this method, following step (b) but prior to step (c), the culture can be incubated for a time and with an amount of a hyaluronidase, a collagenase or both, sufficient to remove or disrupt the glycocalyx coat of said catenae. Such treatments are typically done for about 5-30 minutes at 37° C., and preferably for about 10 minutes. These enzymes do not need to be removed for the duration of the remainder of the assay. Modified and PEGylated versions of the enzymes can also be used in the methods of the invention. These assays can also be readily adapted to an HTS format as above. To determine whether a test compound(s) effects proliferation the cells can be counted manually with or without staining or a fluorescent signal, a luminescent signal or absorbance measured. Because the catenae exist in suspension, detection methods need to be adapted accordingly and can be done by those of skill in the art. One preferred detection method is using alamarBlue® staining, followed by measuring fluorescence or absorbance of the culture which is proportional to the live cells present in the culture and is independent of whether the cells are adherent or in suspension.

A similar assay system for serosal spheroids is also provided. For spheroids, the dissociated cells are cultured for a time and under conditions to produce spheroids of sufficient number and size with an established glycocalyx coat. Because spheroids are large aggregates of many cells, it takes longer to reestablish the coat than it does for catenae. The time frame for spheroids is typically from about 8 to about 14 days, so that adding test compounds is done in that time frame, and preferably at 11 days post seeding.

Hence, these methods allow for screening compounds for their toxicity and their chemical properties against serosal (including ovarian) cancer stem cells (catenae) with their protective pericellular coat undisturbed and represent an in vitro system that is more relevant to the clinical setting than conventional screening methods. The in vivo and in vitro data suggest that catenae are ovarian cancer stem cells adapted to grow in suspension in ascites fluid and that glycocalyx formation, without be limited to a mechanism, might be necessary for growth and expansion of cancer stem cells in ascites fluid and to remain as cancer stem cells. The data also explains the resistance to therapy in advanced stage ovarian cancer with peritoneal metastasis and other serosal cancer types. Any compound identified as toxic to catena with intact pericellular coat in this screen is potentially useful in treatment of advanced stage ovarian cancer.

8. Treatment Methods

A. Targeting the Glycocalyx

The catena's glycocalyx coat of hyaluronan is a predominant morphological feature. Targeting this feature for removal, provides a method of treating serosal cancer, maintaining cancer in a manageable disease state, eradicating cancer stem cells after or during other standards of cancer care (e.g., in conjunction with chemotherapy or radiation treatment) as well as prolonging the time to relapse or metastasis.

Hyaluronan and/or other glycocalyx components may be targeted through a variety of paths including degradation of hyaluronan, prevention of hyaluronan binding to its receptors (for example: CD44, RHAMM), prevention of hyaluronan export or proteins that interact with hyaluronan (for example: Aggregan, Versican). Additionally, hyaluronan expression may be inhibited or reduced by targeting synthetic pathway components which produce hyaluronan by various techniques including RNAi or antisense or addition of enzyme inhibitors. Hyaluronan synthesis can be disrupted by inhibiting formation of parts of its chemical structure (for example: targeting the repeating disaccharide units or the glycosidic bonds). Further, inhibition of hyaluronan synthesis may be accomplished by targeting hyaluronan synthase (HAS) on a DNA, RNA, or protein level (e.g., enzymatic inhibitors). Examples HAS inhibitors include, but are not limited to, 4-methylumbelliferone (4-MU or MU), 4-methylesculetin (ME), brefeldin A, mannos, siRNA against hyaluronan synthase enzymes, antibodies against extracellular or intracellular domains of hyaluronan synthase enzymes, and hyaluronidase (bacterial or animal origin, natural or recombinant) as well as PEGylated or chemically modified derivatives of any of any of the foregoing (as appropriate).

Hyaluronan can be targeted for degradation or removal by antibodies, small molecules, enzymes or other means. Hyaluronan is most commonly degraded by hyaluronidase, a glycoprotein. Hyaluronidase has been recognized as having a potential therapeutic use in cancer. This enzyme or modifications that can be used in animals may be used here for the first time to selectively target serosal cancer stem cells. For example, ovarian cancer is commonly treated with standard therapies including surgery, chemotherapy, radiation, or a combination of these. Such treatment may include platinum based therapies, topotecan, oral etoposide, docetaxel, gemcitabine, 5-FU, leucovorin, liposomal doxorubicin.

The present invention provides for supplementation of these treatments with course of treatment to remove or inhibit glycocalyx formation. For example, in one treatment regimen, the primary cancer is removed (by any means or treatment), followed by hyaluronidase treatment to eradicate any catenae or CSCs that are resistant or escape treatment. Hyaluronidase treatment can also be done concurrently with standard courses of cancer treatment. Further these two therapeutic modalities can be followed by additional rounds of standard therapy (e.g., chemo) if needed.

The invention contemplates other methods of care that eradicate, disrupt morphology, force differentiation, or decrease the clonogenicity of the catena which include hyaluronidase treatment as part of the treatment.

Certain embodiments of the invention provide methods to treat serosal cancer in a patient undergoing a chemotherapeutic regimen or radiation treatment which comprises administering a hyaluronan synthase inhibitor, another inhibitor of the hyaluronan pathway, or an enzyme that degrades hyaluronan, for a time and in an amount to augment or supplement the regimen or treatment or to improve survival time of the patient. The inhibitor can be administered before, after or simultaneous with the chemotherapy regimen or radiation treatment. This method can be followed by additional rounds of chemotherapy or radiation.

The present method leads to cause remission of cancer symptoms, e.g., including tumor regression, less bloating or ascites formation. These methods also inhibit cancer stem cell self-renewal and/or formation in a patient, without being bound to a mechanism, by inhibiting glycocalyx formation by said CSC which thereby inhibits self-renewal and causes differentiation of the CSC. This differentiation may then make the cells again susceptible to standard cancer treatment regimens know in the art.

Serosal cancers, include but are not limited to, ovarian cancer and any cancer that appears in the serosal cavity, whether of primary or secondary (e.g., metastatic) origin.

Enzymes that catalyze hyaluronan breakdown (degrade hyaluronic acid) include the hyaluronidases (e.g., EC 3.2.1.35). Humans have six associated genes, including HYAL1, HYAL2, HYAL3, HYAL4, MGEAS and PH-20/SPAM1. Any hyaluronidase can be used in the invention. A preferred hyaluronidase for use in the present invention is recombinant human hyaluronidase Hylenex (Halozyme Theraputics) derived from the gene PH20. Pegylated PH20 hyaluronidase is also useful.

Hyaluronidase can be of human, other animal or bacterial origin, as well as artificially made (recombinant/synthetic). It may be modified (pegylation, addition of a transporter of oligomers, other commonly known ways to modify an enzyme) and can be provided in any formulation that delivers an effective dose to a patient. Methods of determining dosages and formulating chemotherapeutics are known to those of skill in the art.

In another aspect, the invention is directed to a method to inhibit cancer stem cell self-renewal or formation in a patient which comprises administering an inhibitor of glycocalyx formation or an agent that degrades glycocalyx for a time and in an amount to said patient to inhibit glycocalyx formation or degrade the glycocalyx of CSC in the patient and thereby inhibit self-renewal or formation of said CSC, to cause differentiation of the CSC, to make the CSC susceptible to killing by other chemotherapeutic regimens, or to prevent catena from undergoing spheroid formation.

The inhibitors and enzymes used in the methods of the invention can be provided as pharmaceutical compositions for intraperitoneal or intraserosal delivery in the form of injectable sterile solutions, suspensions or other convenient preparation. Intraperitoneal delivery is particularly useful. When administered orally, the inhibitors and enzymes can be, for example, in the form of pills, tablets, coated tablets, capsules, granules or elixirs. Administration can also be carried out rectally, for example in the form of suppositories, or parentally, for example intravenously, intramuscularly, intrathecally or subcutaneously, in the form of injectable sterile solutions or suspensions, or topically, for example in the form of solutions or transdermal patches, or in other ways, for example in the form of aerosols or nasal sprays. Depending on the nature of the administration, the pharmaceutical compositions may further comprise, for example, pharmaceutically acceptable additives, excipients, carriers, and the like, that may improve, for example, manufacturability, administration, taste, ingestion, uptake, and so on.

B. Other Treatment Methods

Other treatment methods of the invention include a method to treat a serosal cancer which comprises (a) administering an anticancer regimen to a serosal cancer patient; (b) reviewing the results from one or more of the methods in section 5 above performed periodically with samples from said patient, and (c) altering the treatment regimen as needed and consistent with the information provided from those methods, i.e., by monitoring the serosal cancer stem cells present in a patient, a medical practitioner can make informed and personalized decisions about which therapeutic regimens would apply to that particular patient.

9. Potential Therapeutics

In addition to the gene signature information for catena, gene expression analysis gave significant information on the molecular pathways active in catena cells. Based on this information, Table 1 provides a list pathways active in catena and compounds that target those pathways as potentially effective therapeutics for serosal CSCs, and more particularly for ovarian CSCs. Underlined compounds have been tested for efficacy against catenae.

TABLE 1
Catena Pathway Targeting Compounds

| Pathway | Compounds |
|---|---|
| Rho-ROCK | Y27632 |
| pathway | |
| DNA replication | 5-FU, ARA-C, mitomycin-C |
| c-met pathway | PF-02341066 |
| iNOS pathway | LNMMA |
| ROS pathway | L-buthionine Sulfoximine |
| ABC Transporters | Verapamil, Ningalin, Dexverapamil, SDZ PSC 833, SDZ 280-446, |
| | XR9051 |
| | GF120918, Nifedipine, Trifluoperazine, Midostaurin, Thapsigargin |
| | Zaprinast, MK-0457. |
| Metabolic inhibitors | Lovastatin acid, SB-201076, SB-204990, dichloroacetic acid/DCA, |
| | 2-deoxy-D-glucose (2DG), 3-bromopyruvate, 3-BrOP, 5-thioglucose, |
| AKT | Deguelin, GSK690693, MK-2206, Perfosine, Archexin, Triciribine, |
| | OSU-03012, INCB028060, PHT-472, AZD6244. |
| Cell cycle protein | PD-0332991, Olomoucine, Seliciclib., CEP-3891, CHIR-124, XL844, |
| inhibitors | PF-477736, UCN-01, LY2603618, AZD7762, CBT501, SCH 900776, |
| | Kinetin riboside |
| Receptor TK | Dasatinib, Sunitinib, Erlotinib/Tarceva, Nimotuzumab, |
| inhibitors | Cetuximab/Erbitux, Panitumumab, Trastuzumab, |
| | ZalutumumAb, PF-299804, AEE788, Vandetanib, JNJ-26483327, |
| | CI-1033, Lapatinib, PD-158780, BMS-599626 |
| | BMS-690514, PD153035, BIBW2992, ARRY-334543, AG1478 |
| | CL-387785, HKI-272, EKB-TKI, AZD8931. |
| | U0126, Sorafenib, PD0325901, INCB028060, |
| | TK1258, Maiatinib, Danusertib, SU6668, Regorafenib, PHA-665752, |
| | FP1039, AS703569, PD173074. |
| | 19D2, AMG-479, AVE-1642, BIIB022, Figitumumab, Di-diabody, |
| | H7C10, H710, MK-0646, R1507, BioG, IMC-A12, m610, BMS- |
| | 536,924, BMS-554417, EXEL-228, insm-18, NVP-ADW742, NVP- |
| | AEW541, OSI-906, Picropodophylin, PQ401, TAE226, BMS-754807, |
| | SU11274, A-923573, IGF1R antisense, IGF1R interference, m610, |
| | AZD6244, GSK1904529AXL-228, A-923573, INCB028060, 17- |
| | AAG, PU-H71 |
| | BMS-554417, OSI-906, BMS-754807, GSK1904529A, Capecitabine |
| | Etaracizumab, MEDI-522, Volociximab, Natalizumub, Cilengitide, |
| | S247, |
| | Cediranib, CHIR-258, Masitinib, Motesanib Diphosphate, Pazopanib |
| | Hydrochloride, Tandutinib, Vatalanib, Sunitinib Malate, Kit Mab, |
| | Axitinib, Imatinib Mesylate, Midostaurin, WBZ_4, Nilotinib, IMC- |
| | 41A10 |
| FSCN1/Fascin | Migrastatin, 2,3-dihydromigrastatin, Migrastatin core, Migrastatin |
| | ether |
| HAS2, Hyaluronan | 4-methyl-umbelliferone, 6,7-dihydroxy-4-methyl coumarin |
| | Hyaluronidase, rHuPH2, PEGPH20, Zaprinast, Brefeldin A, Mannose, |
| | 4-methylesculetin,5,7-dihydroxy-4-methyl coumarin. |
| HDAC | SAHA, Belinostat, JNJ-26481585, LA0824, Panobinostat, |
| | Mocetinostat, |
| | Entinostat, PCI-24781, Trichostatin A, Vorinostat, SB939, Valproic |
| | Acid. |
| Hedgehog pathway | Cyclopamine, BMS-833923, GDC-0449, IPI-926, LDE225. |
| Heat shock protein | 17-AAG/Tanespimycin, Geldanamycin, 17-DMAG/Alvespimycin, |
| inhibitors | CNF-1010, IPI-504, IPI-493, KW-2478, KF25706, |
| | Cycloproparadicicol, |
| | Radicicol, Pochonin, PU24FC1, PU-DZ8, PU-H71, CNF-2024, SNX- |
| | 5422 |
| | STA-9090, VER-00063579, VER-49009, VER-50589, VER-52296 |
| | G3129, G3130, NMS-E973, PF-04929113, SNX-210, PU-H71, |
| | KU175 |
| | Celastrol, ATI3387, MPC-3100, AUY922. |
| | MAL3-101, VER-155008, Quercetin, KNK437 |
| MEK Targeting | 17-AAG, AMG 102, TAK-701, SCH-900105, XL-184, JNJ- |
| | 38877605, GSK1363089, PF-04217903, PF-2341066, PHA-665752, |
| | SGX-523, SU11274, Compound 1, INCB028060, Foretinib, |
| | INCB028060, h224G11, MGCD265, PU-H71, NK4, MK-2461 |
| mTOR Targeting | Rapamycin, KU-55933, PI-103, Temsirolimus, BEZ235, Deforolimus, |
| | Everolimus, U0126, 852A, Imiquimod, XL765, Palomid 529, |
| | AZD8055, XL765, NVP-BEZ235, BGT226, GDC-0980, SB2312, |
| | PKI-402 |
| NF-kB Targeting | Parthenolide, PDTC, Disulfiram, Olmesartan, Dithiocarbamate |
| Notch/Gamma | DAPT, RO4929097, (Z-LL)2-ketone, L-852646, MRK-003, GSI-I, |
| secretase Targeting | GSI-IX, GSI-XII, GSI-18, GSI-34, LY-411,574, JC-34, JC-22, JC-22, |
| | MK-0752, IL-X, NLT1, NTL2, OMP-21M18, Dibenzazepine, z-Leu- |
| | leu-Nle-CHO, Notch3 siRNA, Begacestat |
| PDGFR targets | Dasatinib, JJ-101, Motesanib, Axitinib, Semaxanib, Sorafenib |
| | Tosylate, SU6668, Sunitinib, Masitinib,, Pazopanib, Regorafenib, |
| | Linifanib, CHIR258, ABT-869, BIBF1120, CHIR-258, Imatinib, |
| | Mesylate, Tandutinib, Vatalanib, Leflunomide, Midostaurin, |
| | CP673,451, IMG-3G3, 2C5, 1 E10 |
| PI3K Targeting | LY294002, GDC-0941, GDC-0980, KU-55933, OSU-03012, PI-103, |
| | XL765, XL147, ZSTK474, AS041164, Deguelin, Halenaquinone, |
| | IC486068, PX-866, SF1126, WAY-266175, Wortmannin, BEZ235, |
| | XL765, NVP-BEZ235, BGT226, BKM120, CAL-120, SB2312, |
| | GSK2126458, PKI-402, Myoinositol, I3C, QLT0267 |
| Proteasome | Bortezomib/Velcade, NPI-0052, MG-132, Celastrol, CEP-18770, PF- |
| Inhibitors | 3084014, MLN9708, PR-047 |
| RAF (A-RAF, B- | 17-AAG, GDC-0879, Sorafenib Tosylate, PLX4032, XL281, RAF264, |
| RAF, C-RAF) | PU-H71 |
| Targeting | |
| SRC targeting | Dasatinib, PHA-665752, Saracatinib, Bosutinib, XL-228, AS703569 |
| Topoisomerase | Doxorubicin, Etoposide, 9-AC, Irinotecan, Camptothecan, 10- |
| (TOP1, TOP2) | Hydroxycamptothecin, 9-methoxycamptothecin, AR-67, Topotecan, |
| Targeting | NK012, Amsacrine, Teniposide, ICRF-193, Thaspine, Artemisini |
| Tubulin-alpha, beta | Epothilone B, dEpoB, 9,10-dehydro dEpob, Fludelone, Iso-oxazol |
| Targeting | fludelone, Paclitaxel, ABT-751, AVE8062, CA4P, DMXAA, |
| | EPC2407, MN-029, TZT-1027, ZD6126, BMS-247550, Patupilone, |
| | KOS-862, BMS-310705, ZK-EPO, KOS-1584, KOS-1584, Docetaxel, |
| | Taxotere |
| VEGFR, VEGF | Sunitinib, Avastin, IMC-18F1, IMC-1121B, PHA-665752, Axitinib, |
| Targeting | Midostaurin, Semaxanib, Sorafenib Tosylate, SU6668, SU6668, |
| | Pazopanib, BIBF1120, CHIR-258, Motesanib Diphosphate, Sorafenib |
| | Tosylate, Vatalanib, E-3810, AG13736, PTC299, Regorafenib, JJ-101, |
| | Brivanib, Linifanib, MGCD265, XL-184, Cediranib, Elesclomol, |
| | Enzastaurin, Vandetanib, XL-184, Vadimezan, GSK1363089, BMS- |
| | 690514, BMS-844203, Tivozanib, Midostaurin, RAF264, MGCD265, |
| | Aflibercept, CEP-3891, MK-2461 |

10. HAS2 Mutations, PFGRA Mutations and HAS2 Splice Variants

HAS2 and PDGFRA are the most highly expressed genes in Ovcar3 catenae. It has unexpectedly been discovered that the HAS2 gene occurs as a splice variant in catenae, that mutations are found in the HAS2 and PDGFRA genes in catenae and in patient tumor samples.

Accordingly, this invention provides isolated nucleic acid encoding a mammalian HAS2 splice variant, including mRNA and cDNA therefor as well as nucleic acids comprising a contiguous nucleotide sequence, in 5′ to 3′ order, that consists essentially of the entirety of or a portion of exon 2 and the entirety of exon 3 of a HAS2 gene, i.e, splice variants that lack exon 1. One mRNA HAS2 splice variant encodes a protein that begins at amino acid 215 of the wt human HAS2 and ends at the normal stop signal, i.e., amino acid 552. The invention also includes vectors comprising any of the nucleic acid of the invention, cells comprising these vectors, as well as using recombinant expression systems produce the encoded proteins, and the encoded proteins. Other embodiments of the invention are directed to isolated nucleic acid probes that are for specific for detecting a mammalian HAS2 splice variant RNA or any one or more HAS2 mutations, including SNP mutations, and preferably detect the mutations identified in Table 17 and 18. The invention thus also includes mutant and allelic forms of the wt HAS2 and HAS2 splice variants.

Yet another aspect of the invention is drawn to a method of monitoring and/or staging serosal cancer in a subject which comprises (a) preparing catenae from ascites obtained from a cancer patient; (b) detecting whether the catenae have one or more HAS2 mutations and/or express one or more HAS2 splice variants; and (c) correlating those mutations and/or variants with the presence and/or progression of cancer in a said patient. Further, one can identify or monitor for the presence of serosal cancer stem cells in a patient sample by (a) obtaining a cellular sample from a patient; (b) optionally, depleting that sample of leukocytes; (c) preparing DNA, RNA or both from the remainder of the sample; and (d) detecting whether the DNA, RNA or both has a HAS2 mutation or expresses a HAS2 splice variant, with the identification of a mutation or a splice variant indicating the presence of serosal cancer stem cells in the sample. By quantitating the amounts of such DNA or RNA, one can correlate the findings with the presence of serosal cancer and/or progression of a serosal cancer in the patient.

These correlations include the ability to make an original diagnosis for the presence o of serosal cancer, early detection of the cancer and its disease stage, the presence of cancer stem cells, the catenae content of a tumor, the aggressiveness of a tumor, the metastatic potential of a tumor and, the risk of metastasis of a tumor. Likewise, the HAS2 status of a patient can be used to stratify patients for hyaluronidase combination therapy and to correlate disease-free survival and response to therapy. A HAS2-based PCR assay can be integrated in clinical trials to follow the effect of chemotherapy on cancer stem cells and determine at early stages of the trial if the therapy is effective or not.

Samples for such assays can be ascites, preferable, but peripheral blood can be used as well. DNA or RNA can be directly amplified form ascites or blood samples and used in PCR method. Specific FISH (fluorescent in situ hybridization) probes for WT and variant mRNA can be used on blood smears or ascites samples spun on a diagnostic slide. The presence of these probes in the same cells can also be determined

The HAS2 splice variant appears to be expressed in more of the ascites samples than solid tumors. Clinically, having ascites is poor prognosis so there is a correlation between variant expression and clinical outcome.

It will be appreciated by those skilled in the art that various omissions, additions and modifications may be made to the invention described above without departing from the scope of the invention, and all such modifications and changes are intended to fall within the scope of the invention, as defined by the appended claims. All references patents, patent applications or other documents cited are herein incorporated by reference in their entirety.

### EXAMPLES

### Example 1

### Development of in vivo Orthotopic Ovarian Cancer Model

The Ovcar3 cell line (obtained from the NCI, NCI-60 panel) was initially derived from the ascites fluid of a patient with an advanced stage of ovarian adenocarcinoma with peritoneal metastasis [Hamilton, 1983]. Cell lines were maintained in M5-FCS media.

Luciferase and green fluorescence protein-expressing Ovcar3 was derived by transduction with a retroviral vector expressing an eGFP-HSV-TK-luciferase (GTL) fusion gene [Ponomarev, 2004]. Transduction efficiency was ˜10%. Transduced Ovcar3 cells were sorted for the highest GFP expression by FACS at the Flow Cytometry Core Facility (MSKCC). GFP-sorted Ovcar3 cells are termed Ovcar3-GTL. Ovcar3-GTL cells were maintained in M5-FCS media. Ovcar3 [symbol] GTL formed epithelial monolayers on tissue culture-treated plates.

Bioluminescence imaging was performed by anesthetizing mice with isoflurane (Baxter Healthcare), and administering d-luciferin (Xenogen) in PBS at a dose of 75 mg/kg of body weight by retroorbital injection. Imaging with a charge-coupled device camera (IVIS, Xenogen) was initiated 2 min after the injection of luciferin. Dorsal and/or ventral images were acquired from each animal at each time point to better determine the origin of photon emission. The data were expressed as photon emission (photons per second per cm2 per steradian). Statistical significance was determined by using Student's t test. Statistical analysis of the luciferase bioimaging model was generated by comparing the area under the curve (AUC) of photon emission between groups of 3-5 mice using the two-sample Wilcoxon rank sum test.

An intraperitoneal (i.p.) injection strategy was chosen to establish a system as close as possible to the clinical manifestation of late stage ovarian cancer, as well as one representing the site from which the Ovcar3 cell line was originally derived. For this xenograft model, NOD-SCID mice, 10- to 12-wk-old females, were injected i.p. with 10×10⁶ Ovcar3-GTL cells. Ovcar3-GTL monolayer cells were dissociated to single cells with 0.05% trypsin in 0.02 EDTA treatment for 5 min at 37° C. (Mediatech) before injection. Mice were treated with i.p. injections of PBS three times per week. The tumor distribution was followed by serial whole-body noninvasive imaging of visible light emitted by luciferase-expressing Ovcar3-GTL cells, upon injection of mice with luciferin.

Due to the need to inject large numbers of tumor cells (5-10 million) to get tumor development and the indolent nature of the tumor growth caused by residual immunity in NOD-SCID mice, more immunosuppressed mice were used for further experiments.

NOD-SCID IL2R gamma −/− (NSG) mice have been developed as a more immunosuppressed strain than NOD-SCID mice. NSG mice lack Natural Killer (NK) cells as well as T and B lymphocytes. Since residual immunity in NOD/SCID mice may have interfered with growth of human cancer cells, NSG mice were compared with NOD/SCID mice in human ovarian cancer xenograft experiments. When Ovcar3-GTL cells were injected i.p. into NSG mice, engraftment was obtained with as few as 25,000 cells. This is 200-fold better engraftment compared to NOD-SCID mice. Moreover, the intraperitoneal tumor growth was followed for months, and eventually mice showed distended abdomen, indicative of ascites formation, together with weight loss. These observations showed that the in vivo mouse model recapitulated many aspects of ovarian cancer with peritoneal metastasis as seen in the clinic.

Sublethal irradiation of NSG mice prior to cell inoculation had a negative effect on the tumor engraftment. It was observed that without irradiation, engraftment of Ovcar3-GTL cells was directly proportional to the number of cells injected. However, sublethal irradiation of mice with 300 Rad prior to cell injections resulted in the same level of engraftment regardless of number of cells injected.

Using NSG mice instead of NOD-SCID mice was a major technical advance for the engraftment efficiency and significantly overcame the issue of antitumor activity of residual immunity of NOD-SCID mice. With higher engraftment efficiency in NSG mice, this orthotopic system provides an excellent model for early stage ovarian cancer and allows one to follow the development of disease to later stages.

### Example 2

### Inflammatory Responses Stimulate Tumor Growth

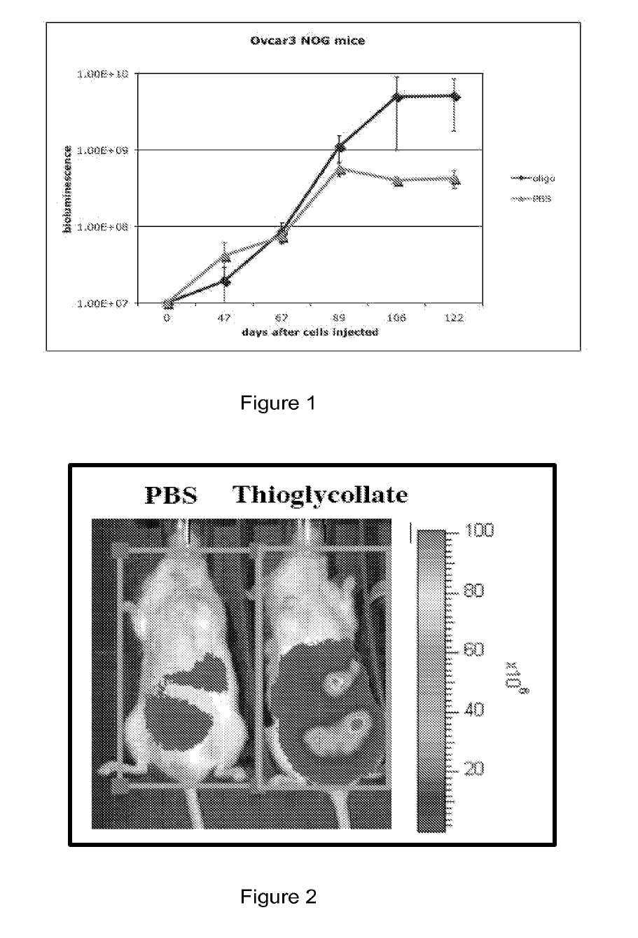

When NSG mice were transplanted i.p. with Ovcar3-GTL cells and injected i.p. with PBS every 3 days for 13 weeks, intraperitoneal tumor growth reached “an equilibrium” as shown in FIG. 1. Once in the equilibrium state, tumor size was maintained at the same level for months in NSG mice. However, in the ovarian NSG model, peritoneal tumor growth was always more rapid and extensive with a greater volume of ascites in the group injected with a lipidated N3′→P5′ phosphoramidate oligonucleotide (“Oligo”) compared to PBS injected group (FIG. 1). The Oligo is a 13-mer having the structure and sequence: 5′-palmitoyl-TAGGTGTAAGCAA-3′.

A BLAST search for the Oligo sequence found matches to a number of murine and human genes. Thus it is possible that the tumor promoting effect of the Oligo in vivo was due to some change in expression of genes in tumor cells or in cells of the mouse peritoneal environment. Alternatively, the repeated injection of the lipidated material may be eliciting a classic inflammation involving peritoneal macrophages. If it is an inflammatory response caused by the lipid moiety of the mismatch compound, another inflammatory exudate, such as thioglycollate, should also increase intraperitoneal tumor growth. To test this, NSG mice were injected i.p. with 10⁶ Ovcar3-GTL cells and 4 weeks later injected i.p. with 1 mL fluid thioglycollate (Hardy Diagnostics) or PBS. Tumor growth in the thioglycollate-treated mice was increased compared to PBS-treated mice (FIG. 2). These results suggest that induction of inflammation in the peritoneum facilitates intraperitoneal ovarian tumor growth.

### Example 3

### Isolation of Tumor Cells from NSG Ascites and Identification of Catena

Peritoneal ascites from ovarian cancer patients is documented to contain tumor cells [Bardiès, 1992; Becker, 1993; Filipovich, 1997; Makhija, 1999], suggesting that ascites from NSG mice with intraperitoneal tumors should also contain tumors. To determine if tumor cells were present, tumor-bearing animals from the Oligo-treated group of Example 1 were sacrificed for analysis of the tumors and the composition of ascites. NSG mice treated with the Oligo developed solid tumors (omental cake) attached to the peritoneal wall and hemorrhagic ascites.

Ascites was harvested from mice with distended abdomen by peritoneal lavage with 5 ml of PBS. The ascites from Oligo-injected mice contained large, free-floating spheroids which settled down to the bottom of a conical tube after 5 minutes of incubation at room temperature. Cancer spheroids are frequently observed in clinical ascites samples from ovarian cancer patients and have been shown to contain cancer stem cells [Szotek, 2006; Zhang, 2008; Bapat, 2005; Bardiès, 1992; Becker, 1993; Filipovich, 1997; Makhija, 1999]. Tumor spheroids are also linked to chemotherapy and radiation therapy resistance of tumors [Gorlach, 1994; Bjorge, 1997; Chignola, 1995; Tunggal, 1999; Olive, 1994].

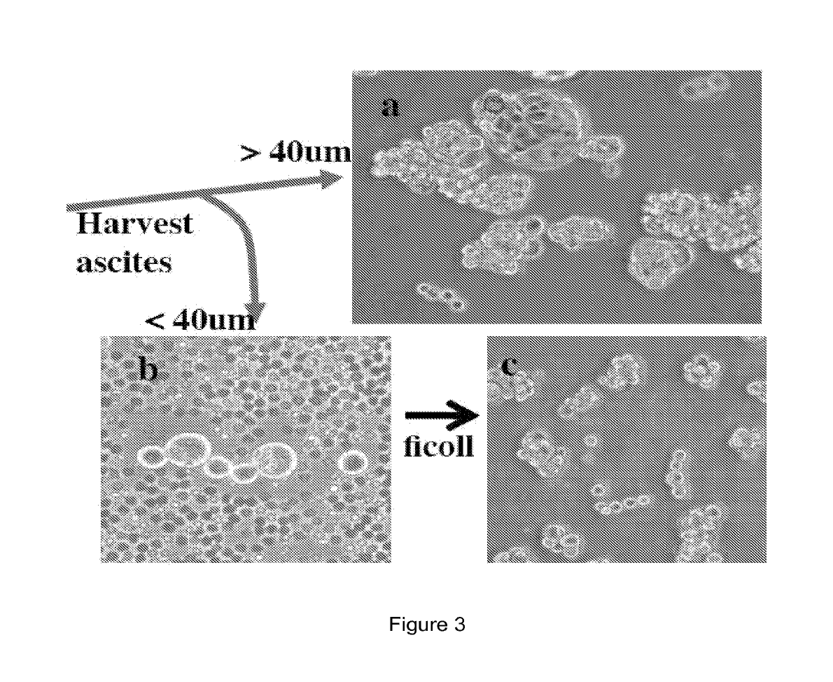

To test whether the spheroids in the ascites of NSG mice bearing Ovcar3-GTL cells contained tumor cells as well as CSCs and to isolate the spheroids, ascites fluid was filtered through a 40 μm strainer (BD Falcon) to select ovarian cancer spheroids (>40 μm diameter). The red blood cells (RBCs) and lymphocytes were removed from tumor cells in the flow through fraction (<40 μm diameter) by centrifugation over a discontinuous density gradient using Ficoll (1.077 g/mL, Accu-Prep, Axis-Shield PoC AS). The cellular content of these fractions is shown in FIG. 3.

Most spheroids, having a diameter larger than 40 micrometers, remained on top of the filter and were harvested for subsequent experiments (FIG. 3a). The flow-through fraction contained cellular structures with a diameter smaller than 40 micrometers which led to an unexpected discovery.

The flow-through fraction was observed microscopically to contain free-floating chains of cells composed of 4-8 individuals cells attached to each other and aligned on an axis (FIG. 3b). Chains were surrounded by a protective coat (glycocalyx) extending up to 20 microns from the cell surface. The glycocalyx prevented interactions with RBCs or other types of hematopoietic cells. The individual cells comprising the chains were larger than RBCs and were separated from them on the Ficoll gradient (FIG. 3c).

To determine if the free-floating chains originated from human Ovcar3-GTL cells or from mouse cells, cells were stained with rabbit anti-GFP antibodies and mouse-anti-human vimentin antibodies (Vector Labs). Free floating chains were fixed on poly-L-lysine coated slides (Sigma). Spheroids were paraffin embedded, sectioned and mounted on poly-L-lysine coated slides. After treatment with the primary antibody, the cells were treated with Tyramide Alexa Fluor 568 (Invitrogen) as the secondary antibody and fluorescent images were acquired using the Discovery XT processor (Ventana Medical Systems) and analyzed by MetaMorph 7.0 Software (Molecular Devices). False colors were assigned to positive signals when necessary.

The chains stained positive for both GFP and human specific vimentin indicating that their cellular origin from human ovarian tumor cells. The unusual morphology of these cellular structures, i.e., these chains of tumor cells, represents a novel multi-cellular entity and are termed “catena” [plural: catenae] (from the Latin for chain).

### Example 4

### In vitro Expansion of Catenae

Ovcar3-GTL cells grown in culture without an intraperitoneal in vivo passage normally form adherent epithelial monolayers in the presence of media containing 10% FCS in tissue culture treated flasks. These monolayers did not form free-floating tumor spheroids even with serum-free media on low attachment plates.

When Ovcar3-GTL-derived tumor cells, isolated as catenae or spheroids as described in Example 3, were cultured in vitro under the same conditions, only a fraction of cells attached to the flask to form adherent monolayers. Moreover, the adherent monolayers had mesenchymal morphology instead of epithelial. In addition, groups of cells piled up on these mesenchymal monolayers and the remainder of the cells remained in suspension as free-floating spheroids and catenae. However, instead of discarding the suspension fraction, a culture system was developed to maintain and expand the tumor cells collected from ascites of NSG mice with ovarian cancer.

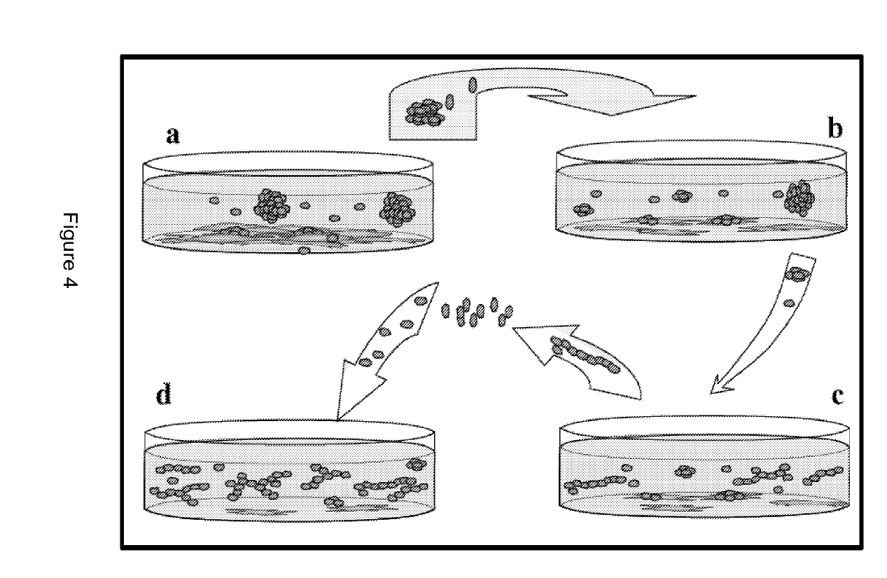

To develop the culture systems, the <40 μm (non-spheroid) fraction and undissociated tumor spheroids (>40 μm fraction) were separately cultured in M5-FCS media (see Example 1) in tissue culture treated flasks (BD Falcon). Suspension cells were collected weekly and filtered through a 40 μm strainer to separate large tumor spheroids from <40 μm fraction, and were passaged into new flasks with fresh media. After 5-6 serial passages of free-floating tumor spheroids, stable spheroid cultures that bred through as free-floating spheroids were established. Similarly, continuous passage of free floating <40 μm fraction generated stable cultures of free floating chains of cells (catenae). A schematic diagram of the suspension culture system is shown in FIG. 4.

After removal of the suspension fractions, the remaining monolayers at each passage were fed fresh media, and a few days after media replacement, groups of cells piling up on mesenchymal monolayers were observed in the attached monolayer cultures. These small, round and refractile cells eventually detached from monolayers and formed new free-floating catenae and spheroids in suspension.

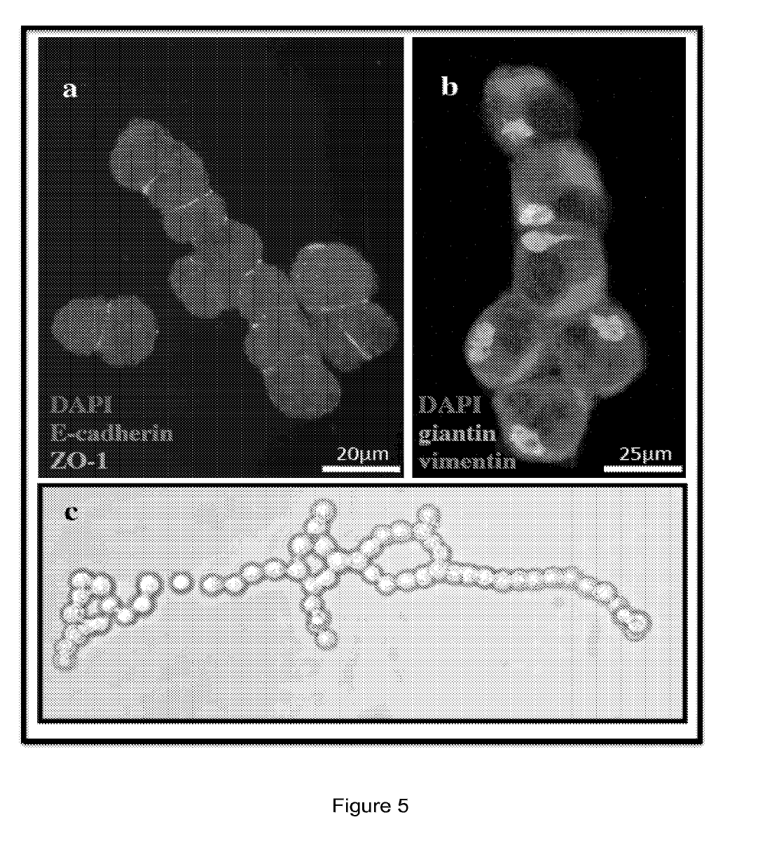

At every passage of the suspension fractions, spheroids and catenae remained in suspension and only a few cells formed monolayers. With increasing passage number, suspension cultures were enriched for free-floating catenae, some generally composed of up to about 72 cells, but were not limited to this exact upper limit (FIG. 5c).

The observation that epithelial Ovcar3-GTL cells became mesenchymal after an in vivo peritoneal passage suggested that the process of development of catenae involved an epithelial-mesenchymal transition (EMT). This phase was followed by small, round and refractile amoeboid-like cells “piling up” on top of the mesenchymal cells (FIG. 4), a mechanism described as mesenchymal-amoeboid transition (MAT) [Friedl, 2003]. The catena transition represents a novel form of cellular transition in which cells remain in suspension, divide symmetrically along the same axis division, and retain ZO-1 tight junctions between the cells.

To test whether catenae formation was the result of aggregation of cells in suspension or of clonal expansion from a single cell by proliferation, catenae were dissociated to single cells by collagenase IV treatment (5 mg/ml collagenase IV (Invitrogen) treatment for 10 min at 37° C.) and cells were followed by time-lapse microscopy for 36 hours using a Perkin Elmer Ultra VIEW ERS Spinning Disk confocal system, powered with MetaMorph image acquisition software. Images were analyzed and movies were created using MetaMorph 7.0 Software (Molecular Devices).

For time-lapse studies, dissociated catenae were seeded in a 96-well plate in M5-FCS media. The plate was then placed under an encapsulated inverted microscope with regulated CO₂ and temperature and was filmed for 48 hours taking images every 10 minutes.

Individual cells were very motile in suspension and observed to repel each other suggesting that catena formation is not caused by aggregation. For example, a 2-cell chain developed into a 9-cell chain by symmetric divisions on the same axis in 36 hours showing that catenae are clonal and cells proliferate rapidly (doubling time <18 hours) to form free-floating chains. Therefore, catena formation is not due to cell aggregation but is a result of clonal and symmetric expansion of suspension cells. It was also observed that a single cell can detach from a catena to form new catenae. The rapid cell cycle progression of catenae did not compromise the linearity of these structures. The division was not restricted to cells at the ends of the chains. Any cell in the catenae could divide often with multiple different cells simultaneously going through mitosis.

To assess the molecular structure of cell-cell junctions in these novel cellular entities, catenae were immunostained with anti-E-cadherin (an adherens junction marker; BD Transduction Lab) and ZO-1 (a tight junction marker; Zymed) (generally as described above). Catenae stained negative for E-cadherin but positive for ZO-1 (FIGS. 5a). Loss of E-cadherin staining suggests that adherens junctions are not involved in catenae formation. ZO-1 staining was localized at the junctions suggesting that catena cells may be attached to each other by tight junctions. Vimentin antibody staining of a catena is shown in FIG. 5c.

During catena formation, a Golgi marker (giantin) localized at the cellular junctions when cells were dividing symmetrically along the “catenal” axis, and at the opposite ends when the division was perpendicular to the catenal axis as shown by immunofluorescent staining using anti-giantin antibodies (FIG. 5c). These experiments showed that the symmetric rapid division of a free-floating single cell along the same division axis formed catenae in which cells remained attached by tight junctions.

### Example 5

### Spheroid Formation

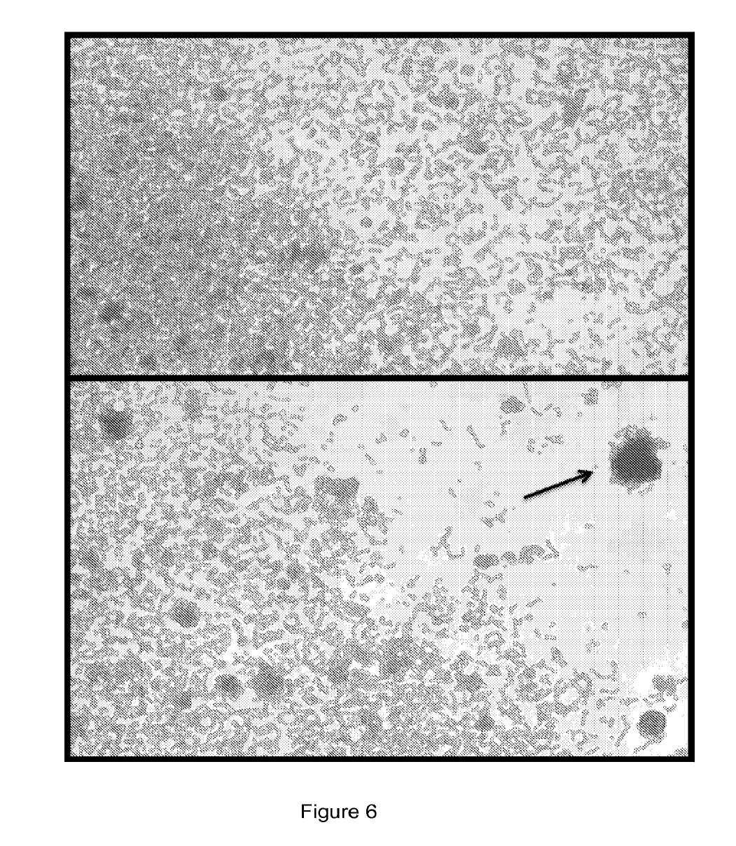

Sub-confluent catenae cultures mostly contained free-floating chains of cells. However, at later stages when there was high density of catenae, free-floating spheroids were observed (FIG. 6). Spheroids developed from catenae by a process of “rolling up,” suggesting that nutrient deprivation at confluent stages of cell culture provided a protective environment for catenae survival.

To understand the interactions between spheroids and catenae, individual spheroids were followed by time-lapse microscopy as described in Example 4. For these experiments, spheroids from suspension culture were dissociated to single cells by collagenase IV treatment (5 mg/ml collagenase IV (Invitrogen) treatment for 10 min at 37° C.). Single sphere forming cells were seeded in a 96-well plate and cultured for 2 weeks prior to microscopy. DIC and GFP fluorescence images were taken every 20 min with constant exposure times for 72 hours.

During the initial stages of spheroid formation, cells amassed on attached mesenchymal monolayers. The cell mass grew in the vertical direction relative to attachment surface, resembling “budding” from attached cells, then developed into spheroids with organized cystic structures. The spheroids eventually detached from attached monolayers and continued to rapidly proliferate in suspension while maintaining the sphere morphology. A schematic diagram of this process is shown in FIG. 7. Developing spheroids were also found to extrude fresh catenae into the suspension. Those catenae proliferated rapidly to form new floating catenae.

Immunofluorescence staining of paraffin-embedded spheroids was done as described in Example 4 using anti-Ki-67 (Vector Labs), anti-phospho-histone H3 (Ser 10) (Upstate), anti-beta-catenin (Sigma), anti-atypical PKC (aPKC), anti-E-cadherin (BD Transduction Lab), and anti-ZO-1 (Zymed) as primary antibodies.

The glandular structures in spheroids are generated by organized movement of cells synchronized with cell division and recapitulated the original Ovcar3-GTL adenocarcinoma phenotype. Most of the cells stained positive for Ki-67 indicating that these cells were actively proliferating. As observed with catenae, spheroids cells were also E-cadherin negative and ZO-1 was detected at the cell to cell junctions. Beta-catenin and aPKC were localized at the cell membrane of every cell in the spheroids. There was a lumen in the middle of the spheroids but apical-basal polarity was not present as determined by homogenous staining of ZO-1, beta-catenin and aPKC in the spheroids instead of their staining being confined to the cells lining the lumen.

These experiments established a biological link between free-floating catenae and spheroids showing that catenae can roll-up to form spheroids and spheroids can extrude catenae into suspension. These morphological states appear dynamic and interchangeable. Catenae and tumor spheroids were initially observed together in the ascites from a mouse injected with human ovarian cancer cells, suggesting that catenae and spheroid formation may be central to the development of ovarian cancer in the peritoneal cavity.

### Example 6

### Catenae and Spheroids Self-Renew

Both Catena and spheroids were derived by an in vivo peritoneal passage of an human ovarian epithelial cell line, Ovcar3-GTL. The extraordinary biology of catena formation by remarkably rapid cell divisions stimulated us to investigate the role of catenae in tumorigenesis.

Previously described ovarian cancer spheroids contain clonogenic CSCs that have extensive self-renewal capacity [Bapat, 2005]. The morphological relation between catenae and spheroids in this study and the observed clonal nature of each catena in suspension culture, suggested a functional link between catenae and cancer stem cells (CSCs).

The clonogenicity of catenae and spheroids was tested in vitro by plating single cells from catenae or spheroids in multi-well cell culture plates. Catenae and spheroids were dissociated to single cells by 5 mg/ml collagenase IV (Invitrogen) treatment for 10 min at 37° C.; Ovcar3-GTL monolayers were dissociated to single cells with 0.05% trypsin in 0.02 mM EDTA treatment for 5 min at 37° C. (Mediatech). Single cell FACS sorting was performed using a MoFlo Cell Sorter. After dead cell exclusion by DAPI, GFP+ single cells were deposited into 96-well tissue culture treated plates (BD Falcon) containing M5-FCS media for Ovcar3-GTL catenae and monolayers or containing serum-free mTeSR1 media (Stem Cell Technology) for Ovcar3-GTL spheroids. Wells were scored visually for growth at day 14 by an inverted phase contrast microscope (Nikon). Colonies from the first clonogenic assay were pooled and dissociated to single cells by collagenase IV treatment and subjected to single cell FACS sorting for the secondary and tertiary in vitro clonogenic assays.

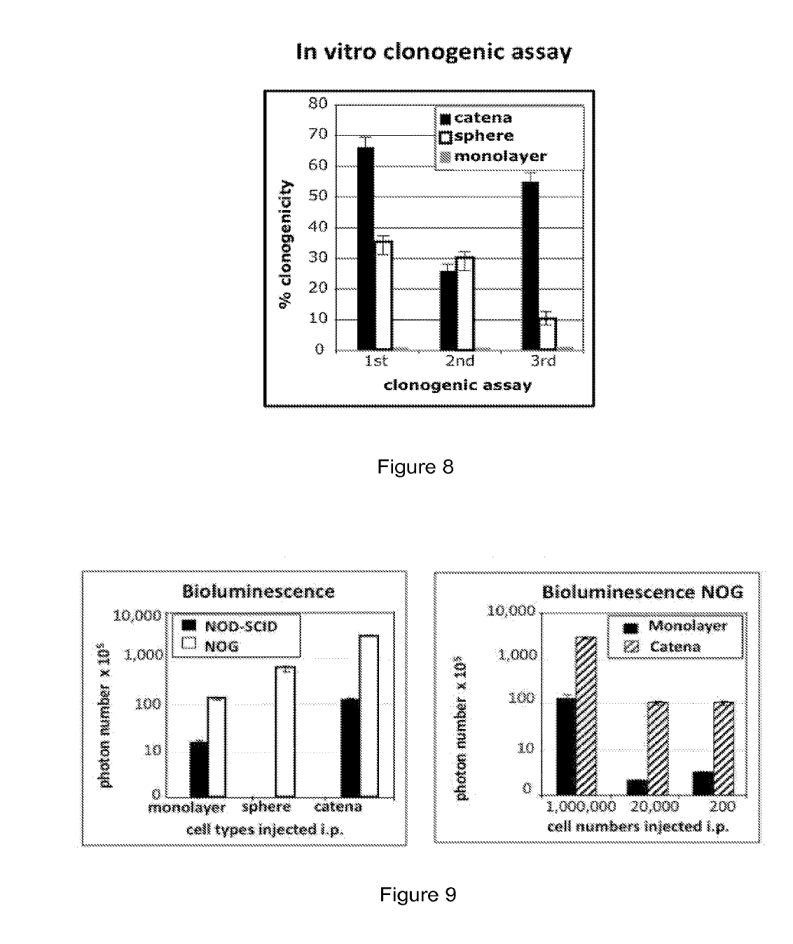

In vitro clonogenic assays showed that catenae were highly enriched for clonogenic candidate CSC since upon dissociation and single cell plating, 55-65% of catenae cells recloned, predominantly forming new catenae (FIG. 8). Recloning potential of spheroid cells was also high (10-30%) and formed new spheroids predominantly, with few catenae. Because single cells from developed spheroids mostly give rise to spheroids, it suggests that a stable modification may control the morphological switch between catenae and spheroids.

Catenae and spheroids have been maintained stably in vitro for 24 months without losing their clonogenicity. Colonies from the first clonogenic assay were pooled and dissociated to single cells by collagenase IV treatment and subjected to single cell FACS sorting for secondary and tertiary in vitro clonogenic assays. This pattern of high clonogenicity persisted by the third single cell recloning passage with catenae forming catenae (recloning potential 55% in FCS-containing medium, 45% in serum-free, ES medium) and spheroids forming spheroids (10% recloning potential). In contrast, when Ovcar3-GTL epithelial monolayer cells were grown as monolayers in FCS-containing medium, 1% of the cells were capable of recloning; whereas in serum-free medium, no recloning was obtained. Monolayer cells were also sorted into Matrigel-coated wells and retained 1% clonogenicity.

These in vitro clonogenic experiments therefore indicate that both catenae and spheroids are enriched for clonogenic cells relative to epithelial monolayers. Catena cells were enriched for clonogenic cells with extensive self-renewal capacity shown by 65% clonogenicity over multiple passages in 24 months.

### Example 7

### Catenae and Spheroids Differentiate in Vivo

A tumor-initiating, limiting-dilution assay in immunodeficient mice was used to assess CSCs in catenae and spheroids.

The CSC nature of catena and spheroid cells was assessed by intraperitoneal transplantation in 8-12 week old female NSG and NOD-SCID mice using 10⁶ cells from the third single cell recloning passage of Ovcar3-GTL catenae and spheroids. In these experiments, groups of three nonirradiated mice were injected i.p. with 10⁶ dissociated catena cells or 10⁶ dissociated Ovcar3-GTL monolayer cells. Another group of nonirradiated NSG mice was injected i.p. with 10⁶ undissociated spheres. Mice were imaged at week 1 and week 2 as described in Example 1. Mice were monitored for distended abdomen and weakness.

For the same number of injected cells, dissociated catenae and undissociated spheroids engrafted better than Ovcar3-GTL monolayer in both NSG and NOD-SCID mice (FIG. 9). Furthermore, all cell types engrafted significantly better in NSG mice than in NOD-SCID mice (FIG. 9, left panel), suggesting that the residual immunity in NOD-SCID mice still plays a negative role on engraftment of highly clonogenic cells.

Similarly, tumor-initiating, limiting-dilution experiments were performed in NSG mice using dissociated OvCar3-GTL catenae and monolayers (FIG. 9, right panel). Groups of three mice were injected with 10⁶, 20,000 or 200 cells. As few as 200 catena cells formed intraperitoneal tumors within 7 days whereas 20,000 monolayer cells did not by 14 days, suggesting enrichment of catena CSCs of at least 100 fold compared to epithelial monolayers. Large tumors were observed within 2 weeks in 3/3 mice injected with 200 catena cells whereas only 1/3 mice had a small tumor in 2 weeks when injected with 20,000 monolayer cells.

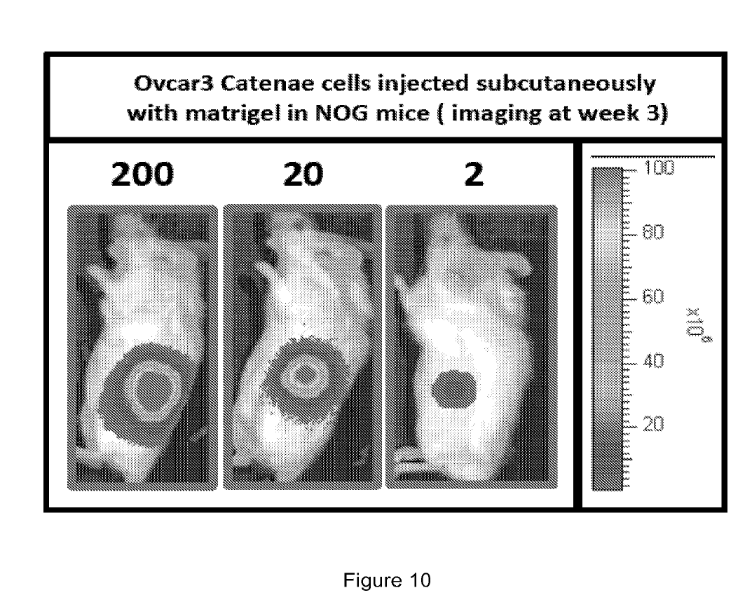

Intraperitoneal injection of 20 catena cells did not result in tumor formation by 6 months. Dilution of autocrine factors in the peritoneal environment could delay the growth of tumors initiated with limiting numbers. To determine if autocrine factors were playing a role and to overcome possible dilution effects, 200, 20 or 2 dissociated catena cells were injected s.c. with 100 μL Matrigel into NSG mice. Bioimaging at 3 weeks showed that 2 catena cells were able to form tumors in a subcutaneous model (FIG. 10). Tumor samples were scored as engrafted when a subcutaneous tumor reached a diameter >0.5 cm.

By suspending catena cells in serum containing media mixed 1:1 with Matrigel, intraperitoneal injection of a single catena cell was able to form a detectable peritoneal tumor in 3 weeks in NSG mice. Similarly, a single catena cell in serum containing media mixed 1:1 with Matrigel injected subcutaneously was also able to form a detectable subcutaneous tumor in 3 weeks in NSG mice. The use of Matrigel in intraperitoneal injections increases the engraftment efficiency.

Morphologically, the resulting ascites spheroids and attached tumors from the above assays maintained the features of serous ovarian adenocarcinoma with defined papillary structures. In spheroids, some cells underwent morphological reversion (differentiation), i.e., a switch from amoeboid to mesenchymal morphology, associated with differentiation and development of complex cyst and duct structures.

These experiments demonstrate that catenae are a novel cellular entity composed of ovarian CSCs with extensive self-renewal capacity (65% clonogenicity over 24 months) and multilineage differentiation potential (complex cyst and duct structures). The unusual cellular morphology of catenae is also associated with its extremely fast doubling time (<18 hours) and high clonogenicity (˜65%).

### Example 8

### An in vivo Metastatic Model with Ovarian Cancer Stem Cells

Intravenous injection of 300,000 GFP/luciferase-labeled catena cells in NSG mice resulted in multiple tumors. By bioluminescence imaging, tumor localization was observed at the femur joints and peritoneum after 6 weeks. Necropsy and histopathology confirmed the presence of neoplastic cells within multiple tissues. Infiltrates in several tissues, such as the liver, were severe enough to interfere with normal organ function. Salivary glands were free of neoplastic cells within the examined tissues; however, neoplasia was present surrounding one of the lower incisors.

The pathological examination showed carcinoma with multifocal mucinous differentiation at multiple topographic sites: There was a moderate amount of yellow, gelatinous fluid in the subcutis. The abdomen was markedly distended. A 0.5 cm diameter, freely-moveable, moderately firm off-white mass was present in the soft tissue adjacent to the right stifle joint. Multifocal, pinpoint to 1 mm diameter, translucent, slightly raised foci were scattered throughout the lung lobes. Normal liver architecture was nearly effaced by disseminated, 0.3 cm diameter to 2.3×1.2×1.2 cm, moderately firm, reddish-tan nodules. There was a scant amount of clear, viscous fluid adhered to the capsular surface of the liver. The right ovary was enlarged, measuring 0.7 cm in diameter. There was a 0.4 cm diameter red focus in the proximal aspect of the right uterine horn. A 1.0 cm diameter, translucent, fluctuant nodule was present adjacent to the cranial pole of the left kidney.

In summary, intravenous injection of catena cells into NSG mice resulted in invasion of ovary and uterus but not fallopian tube; tumor formation and hock and stifle joints; invasion of lungs; large metastasis to liver, viscous material around liver; subcutis yellow edema because of liver dysfunction; and viscous ascites formation.

### Example 9

### Composition of Engrafted Intraperitoneal Tumors

The engraftment experiments in Example 7 showed that both catenae and spheroids were highly enriched in tumor initiating cells compared to differentiated epithelial monolayers. To understand how the morphological difference between catenae and spheroids is reflected in the composition of intraperitoneal tumors they generate, the ascites and solid tumors were analyzed from mice injected with either Ovcar3-GTL catenae or undissociated spheroids. The ascites harvested at week 4 from catena-injected mice contained free-floating spheroids whereas that from mice injected with undissociated spheroids contained significantly fewer free-floating spheroids at week 4. Injection of either catena or undissociated spheroids lead to the formation of omental cakes. These results suggest that catenae and spheroids represent different stages of ovarian cancer development in the peritoneal cavity with extensive proliferation of catenae resulting in spheroid formation which in turn attach to the mesothelial lining and grow as a solid mass into omental cakes.

### Example 10

### Catena Formation from Mesenchymal Ovarian Cell Lines

The in vivo experiments with Ovcar3-GTL monolayers led to the hypothesis that epithelial ovarian cancer cells undergo an epithelial to mesenchymal transition (EMT) followed by a mesenchymal to catena transition to produce catenae and spheroids. In vitro culture of Ovcar3 epithelial cell monolayers did not undergo mesenchymal to catena transition. However, after in vivo peritoneal passage, those cells spontaneously underwent a mesenchymal to catena transition to produce suspension cultures of catena and spheroids when grown under conditions that did not support a mesenchymal to catena transition for monolayers. These results suggest that malignant mesenchymal cells, with genetically stabilized EMT, are capable of a mesenchymal to catena transition, and hence spontaneously producing catenae and spheroids, without need for in vivo peritoneal passage.

To determine if mesenchymal cells can produce catena and spheroids without in vivo passage, i.e., if those cells will undergo a mesenchymal to catena transition spontaneously as peritoneal-passaged Ovcar3-GTL cells did in vitro, the suspension cells in the media from Ovcar5-GL and A2780-G monolayers was serially passaged as described in Example 4 to enrich for catena and spheroids and to develop suspension cultures of each entity.

The Ovcar5 cell line was obtained from the NCI (NCI-60 panel). Luciferase and green fluorescence protein-expressing Ovcar5 was derived by transduction with a lentiviral vector expressing an eGFP-Iuciferase (GL) fusion gene. Transduced Ovcar5 cells were sorted for the highest GFP expression by FACS. GFP sorted Ovcar5 cells are termed as Ovcar5-GL. The A2780-GFP cell line, also designated herein as A2780-G, was provided by Dr D. Spriggs (Memorial Sloan-Kettering Cancer Center).

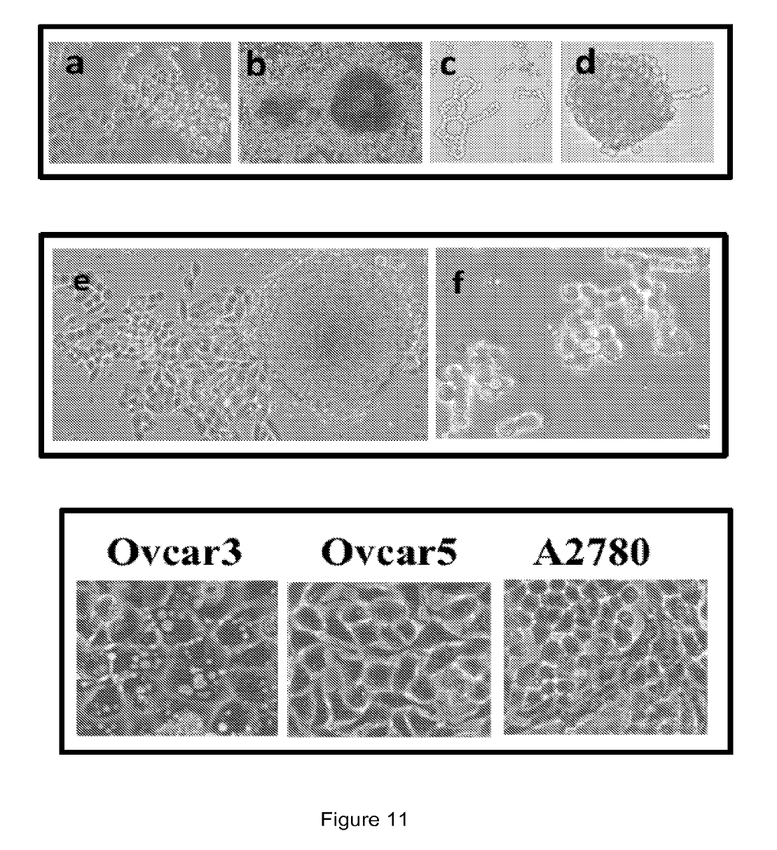

A2780-G and Ovcar5-GL monolayer cell lines were cultured in M5-FCS media in tissue culture treated flasks. Under these conditions, the majority of cells grew as mesenchymal monolayers with a subfraction of free-floating suspension cells. To enrich for catena- and spheroid-forming cells, suspension cells were separated from the monolayers by removing the suspension fraction. Suspension cells were precipitated by centrifugation at 300×g for 5 minutes and resuspended with fresh media. Cells were re-plated into new flasks and suspension fractions were passaged weekly until cultures were enriched for free-floating catenae and spheroids. Hence, the mesenchymal to catena transition occurred spontaneously in vitro without requiring an in vivo passage (FIG. 11). Single cell in vitro clonogenic assays showed that Ovcar5-GL monolayers contained 5% clonogenic cells whereas Ovcar5- GL catenae had 30%.

### Example 11

### Secreted Mesenchymal Monolayer Inhibitory Factor Prevents Catena Self Renewal and Promotes Differentiation

Because suspension fractions from mesenchymal tumor cells had to be passaged several times before a mesenchymal to catena transition occurred, it suggested that the process of serial passaging might be removing or diluting out a possible inhibitory factor that prevented spontaneous catena transition in mesenchymal monolayer cultures. If such a factor (or factors) existed then mesenchymal tumor cells should inhibit catenae in a co-culture system where both types of cells were cultured in the same flasks and the cells constantly secreted such factors.

Catenae were co-cultured with Ovcar5-GL or A2780-G mesenchymal monolayers in transwell plates with a 0.22 μm filter separating the chambers. The mesenchymal cells were placed at subconfluent levels in the bottom chamber and catena cells were placed on the top chamber. Catena growth as free-floating chains in suspension was dramatically inhibited and catenae remained in suspension as single cells or attached to the tissue culture flask and differentiated to mesenchymal cells. If conditioned mesenchymal media was heated to 70° C. and added to catena cultures, the inhibitory activity was lost. These results suggest that differentiated mesenchymal cancer cells secrete a heat-labile, inhibitory factor which prevents uncontrolled expansion of cancer stem cells in suspension.

### Example 12

### Catena Formation from Early Passage Serosal Tumor Cell Lines

The SKOV-6 and CAOV-2 cell lines (from Dr. Lloyd Old, MSKCC) were derived from ascites of patients with papillary serous ovarian adenocarcinoma and had not been passaged extensively before use. Frozen cells from passage 5-10 were thawed and maintained in M5-FCS media. Catena were derived by serial passage of suspension fractions of SKOV-6 and CAOV-2 cell lines as described in Example 4.

In early passage cultures, many round and refractile cells were found piling up on mesenchymal monolayers or as free-floating chains in suspension. Serial passaging of suspension fractions enriched for mesenchymal to amoeboid transition events and catenae formation in CAOV-2 and SKOV-6 cells.

### Example 13

### Catena and Spheroid Formation from Cancer Patient Ascites

### 1. Catena Formation

Serosal cancer samples from pleural, pericardial or ascites fluids containing tumor cells were obtained from cancer patients with metastatic cancer. Tumor cells were harvested by centrifugation at 1200 rpm for 10 min. The serosal fluid was removed and stored at −20° C. The harvested tumor cells were put into tissue culture flasks with serosal fluid from the same patient mixed 1:1 with serum-containing media. Free-floating chains of tumor cells were immediately observable under the microscope. The chains remained in suspension for many weeks. The tumor cells were cultured at 37° C. for several weeks and each week, the free-floating chains of cells in suspension were separated from the attached cells and replated into a new flask with the same combination of serosal fluid and serum-containing media. In these studies, as few as 100 of these free-floating cells from primary serosal tumor samples were able to form tumors in NSG mice in 3 months when injected subcutaneously. When injected intraperitoneally, these cells formed peritoneal tumors in NSG mice in 3-6 months with up to 10 ml of ascites containing free-floating tumor chains, liver metastasis and with solid tumors attached to peritoneal wall. Subsequent in vitro cultures of ascites samples from xenografts identified non-attached free -floating cells.

2. Generation of Spheroids from Catena in Primary Serosal Tumor Samples:

To produce spheroids, catena from primary serosal tumor samples growing in suspension were resuspended in serum-containing media mixed 50:1 with Matrigel and cultured at 37° C. The catena from these primary serosal tumor samples rolled up to form organized tumor spheroids at about 5 days. Cultures were supplemented with serum containing media every week and after 2 weeks, tumor spheroids were observed to extrude catena into culture. Tumor spheroids can be maintained for weeks in vitro with this cell culture method.

### Example 14

### Model of the Catena-Spheroid CSC Concept

The data indicate that catenae are clonally derived and do not develop by aggregation of diverse cell types. Catenae are uniform in morphology and in differentiation state, i.e., they are clonally pure CSCs. While chain migration and a mesenchymal to catena transition are linked to tumor invasiveness, catenae provide a mechanism for rapid, symmetric CSC expansion. CSC expansion does not occur as efficiently in spheroids, and since spheroids contain proportionately fewer CSCs than catenae, it suggests that spheroids may structurally serve to protect CSCs and allow those CSCs to enter quiescence.

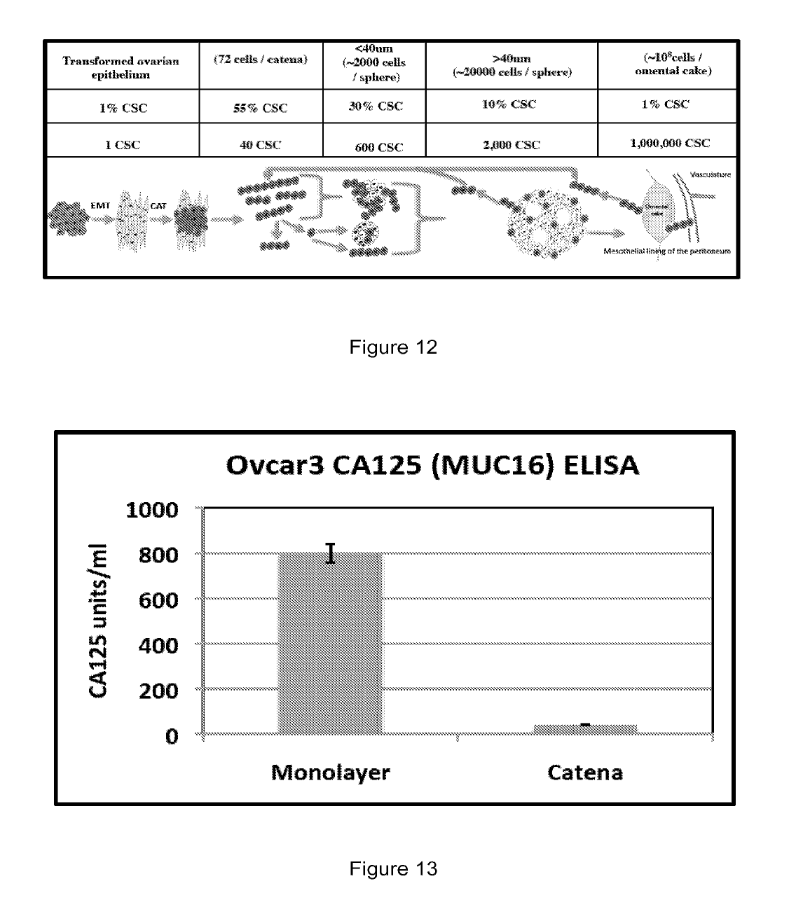

FIG. 12 provides a model of the catena-spheroid concept and the role of CSCs in the development of ovarian cancer. The initial transformation of ovarian (or fallopian) epithelium (green) progresses via an epithelial-mesenchymal and mesenchymal-catena transition. The catena cells (red) lose all attachment to extracellular matrix or peritoneal mesothelium but remain attached to each other following each round of symmetric division. At this point, the catena is composed predominantly of CSCs. The catenae can release single cells that generate secondary catenae or form spheroids. The catenae can also rollup and form spheres which contain a >10 fold higher frequency of CSC than tumors growing as 2D monolayers or solid tumors. Spheroids can release new catenae or can attach to the mesothelial wall of the peritoneum to form omental cakes. Catenae may be released from solid tumors by a mesenchymal-catena transition and may reenter the peritoneal ascites or penetrate into blood vessels leading to more distant metastasis.

### Example 15

### Screening Catena for Drug Sensitivity

### 1. Methods

Ovcar3-GTL-derived catenae were tested for their ability to self-propagate in flat bottom 384-well microtiter plates (Corning). Cultures of Ovcar3-GTL catenae were mechanically or enzymatically dissociated to single cells. For mechanical dissociation, catena cultures were pipetted vigorously, an equal volume of M5-FCS media was added to decrease the viscosity, and the cells were pelleted. For enzymatic dissociation, catena cultures were incubated at 5 mg/ml collagenase IV (Invitrogen) for 10 min at 37° C. followed by centrifugation to pellet the cells. Cells were resuspended in M5-FCS to produce homogenous cultures of single cells which were seeded in 50 μL aliquots per well at the indicated cell densities and grown for the indicated times before addition of test compounds or other reagents.

To assess cell growth, cells were observed under the microscope and manually counted using a hemocytometer or were treated with alamarBlue® by adding 1/10 volume of alamarBlue reagent directly to the culture medium, incubating the cultures for a further 48 hours at 37° C. and measuring the fluorescence or absorbance. Both spectroscopic methods gave comparable results. The amount of fluorescence or absorbance is proportional to the number of living cells and corresponds to the cells metabolic activity. Fluorescence measurement is more sensitive than absorbance measurement and is measured by a plate reader using a fluorescence excitation wavelength of 540-570 nm (peak excitation is 570 nm) and reading emission at 580-610 nm (peak emission is 585 nm). Absorbance of alamarBlue® is monitored at 570 nm, using 600 nm as a reference wavelength. Larger fluorescence emission intensity (or absorbance) values correlate to an increase in total metabolic activity from cells.

Because the components of the pericellular glycocalyx were significantly removed prior to cell seeding by mechanical or enzymatic dissociation of catena, the optimal time for adding compounds to ensure that the catenae had an established glycocalyx was determined and was found to be 3-6 days after seeding. For these experiments, 25 Ovcar3-GTL catena or 250 Ovcar3-GTL catena cells were seeded per well as described above. Test compounds were added at concentrations ranging from 12 pM to 100 μM (across the plate) on days one through six after seeding. Five days after adding the test compound, alamarBlue® was added to the cultures and culture absorbance was measured 48 hours later. No significant difference was observed between 25 or 250 cells in terms of drug sensitivity.

### 2. Proliferation Results

The results are shown in Table 2 for 23 test compounds on OvCar3-GTL catenae. This table sets out the identity of the test compound, the measured IC₅₀ in μM for samples in which the test compound was added one day after seeding (cells predominantly lacking a glycocalyx) and for samples in which the test compound was added six days after seeding (cells having an established or substantial glycocalyx). The final column of the table provides the increased fold of drug resistance from day 1 to day 6.

The results show that catena became resistant to 21 out of 23 agents in 6 days. Only bortezomib (Velcade®) and deguelin showed no differential sensitivity. The formation of glycocalyx in 6 days, for example, conferred more than 8,000,000-fold resistance in catenae to paclitaxel, fludelone and 9-10dEpoB. These results show that adding the compounds 1 day after cell seeding may lead to overestimation of the toxicity of compounds.

Another 6 compounds were tested which did not show any effect on catena cells, even at high concentrations. The compounds, 4-methylumbelliferone (4-MU), Y27632, 9-aminocamptothecin (9-AC), LNMMA, verapamil and dasatinib exhibited an IC50 of 100 μM whether added on day one or day six post-seeding.

The foregoing total of 29 compounds were tested in parallel on ovarian cancer monolayer cells by seeding 100 Ovcar3 monolayer (epithelial) or 25 Ovcar5 monolayer (mesenchymal) cells in 384-well plates. Drugs were added 4 days after cell seeding and cell viability was analyzed by alamarBlue staining. In general, catena cells with an established glycocalyx were on average 4-8 fold more resistant to these compounds when compared to monolayers. However, this resistance was more pronounced for some compounds, including paclitaxel, iso-oxazole-fludelone, fludelone and 9-10dEpoB as shown in Table 3. These four compounds were highly inhibitory to the Ovcar3 and Ovcar5 monolayer cells, having IC50 values ranging from subnanomolar to no more than 50 nM, whereas catena cells (IC50 100 μM) were at least 2000-fold more resistant to these compounds.

The effect of these 29 compounds were also tested on established tumor spheroids. For these assays, 100 spheroid forming cells were seeded in 384-well plates and cultured for 11 days to allow the formation of tumor spheroids before adding drugs. Five days after adding the compound the cells were stained with alamarBlue and scored as above. Overall, spheroids showed the same pattern of drug resistance as catenae with an established glycocalyx. In the case of deguelin, spheroid formation conferred an additional 15-fold resistance to the cells, i.e., catena had an IC50 of 0.025 μM whereas the spheroid IC50 was 0.4 μM.

TABLE 2
Ovcar3-GTL Catena Drug Sensitivity

| | | IC₅₀ (μM) | | |
|---|---|---|---|---|
| | | Addition | Addition | Increase in |
| | | on | on | Resistance |
| | Test Compound | Day 1 | Day 6 | Day 6/Day 1 |
| 1 | paclitaxel | 0.000012 | 100 | 8,333,333 |
| 2 | fludelone | 0.000012 | 100 | 8,333,333 |
| 3 | 9,10 dehydroEpoB | 0.000012 | 100 | 8,333,333 |
| 4 | dEpoB | 0.000400 | 100 | 250,000 |
| 5 | iso-oxazole-fludelone | 0.003000 | 100 | 33,333 |
| 6 | Epo-B | 0.025000 | 100 | 4,000 |
| 7 | topotecan | 0.02 | 100 | 5,000 |
| 8 | Ara-C | 0.05 | 100 | 2,000 |
| 9 | daunarubacin | 0.006 | 0.8 | 133 |
| 10 | etoposide | 0.4 | 50 | 125 |
| 11 | PD-0332991 | 0.6 | 50 | 83 |
| 12 | mitomycin-C | 0.05 | 3 | 60 |
| 13 | 17AAG | 0.012 | 0.4 | 33 |
| 14 | 5-FU | 3 | 100 | 33 |
| 15 | doxorubicin | 0.025 | 0.8 | 32 |
| 16 | PF-02341066 | 0.8 | 25 | 31 |
| 17 | SAHA | 1.5 | 12 | 8 |
| 18 | parthenolide | 3 | 25 | 8 |
| 19 | LY294002 | 25 | 100 | 4 |
| 20 | lovastatin acid | 25 | 50 | 2 |
| 21 | rapamycin | 12 | 25 | 2 |
| 22 | deguelin | 0.025 | 0.025 | 1 |
| 23 | bortezomib | 0.013 | 0.013 | 1 |

TABLE 3
Drug Sensitivity For Monolayers v. Catenae

| | IC₅₀ (μM) | | | Increased |
|---|---|---|---|---|
| | Ovcar5 | Ovcar3 | Ovcar3 | Resistance |
| Test Compounds | monolayer | monolayer | catena | of catena |
| Paclitaxel | 0.0250 | 0.0250 | 100 | 4,000 |
| Iso-oxazole- fludelone | 0.0120 | 0.0500 | 100 | 2,000 |
| Fludelone | 0.0004 | 0.0008 | 100 | 125,000 |
| 9,10 dehydroEpoB | 0.0004 | 0.0004 | 100 | 250,000 |

### 3. Morphological Results

Observing the catena cells under the microscope showed the presence of live, large single cells, i.e., cells arrested at mitosis, in cultures treated with high concentrations of compounds (100 μM topotecan, 25 μM rapamycin, 50 μM lovastatin acid, 100 μM iso-oxazole-fludelone, 100 μM fludelone, 100 μM ara-C, 100 μM 9-10dEpoB, 100 μM paclitaxel). When these cells were harvested and cultured in the absence of drugs, they re-entered the cell cycle.

Catenae treated with rapamycin formed tight spheroids with demarcated edges. These spheroids continued to grow in the presence of high concentrations of rapamycin (>50 uM) and retained their spheroid morphology. The formation of tight spheroids was also observed when catena cells were treated with SAHA (an HDAC inhibitor).

Catenae treated with 5-fluorouracil (5-FU) exhibited a morphological change resulting in formation of fused chains, suggesting that 5-FU may interfere with the tight and adherence junctions of catena. Similar structures were observed in ovarian cancer ascites and metastatic breast cancer patient samples. The change in the cell-to-cell junctions might also be a resistance mechanism where cells activate signaling pathways by increasing cell-to-cell attachment or more tightly control transport of molecules between cells.

Catena cells lost their polarity and formed free floating irregular cell aggregates when treated with high concentrations of verapamil. Similar morphological changes were observed when catena cells were treated with PEGylated or non-PEGylated bovine testis hyaluronidase at day 5 post seeding and cultured until day 10. When the coat is removed/destroyed by hyaluronidase catena cells lose their polarity and form irregular aggregates in vitro.

### Example 16

### Glycocalyx Analysis

The catena and spheroid cultures became increasingly viscous at high cell density. Without passage, the catena cultures became so viscous that harvesting the suspension cells was difficult even after a long incubation with collagenase-IV and/or strenuous mechanical dissociation, suggesting that the presence of a glycocalyx coat around the catenae and spheroids was generating the viscous (or mucinous) media. The cells and culture media were examined for the presence of mucins and hyaluronan.

Initial FACS analysis for the mucin CA125 (the protein product of the MUC16 gene), a biomarker for different types of cancer, indicated that CA125 was not expressed on the surface of catenae. Likewise, ELISA experiments showed that CA125 was not secreted by catenae (FIG. 13). In contrast, Ovcar3-GTL epithelial cells were 98% positive for CA125 by FACS and secreted 800 units/ml of CA125 into culture media. For the ELISA, cell supernatants were collected by spinning the cultures at 300×g for 5 min to remove cells and assayed by CA125 ELISA using an automated instrument, ADVIA Centaur XP Immunoassay System (Siemens Healthcare Diagnostics Inc.).

Hyaluronan is a glycosaminoglycan found in extracellular matrix and functions to provide microenvironmental cues in a number of biological processes, including tumor development [Toole, 2004]. Supernatants prepared as above were treated with a few drops 10 mg/mL hyaluronidase (Sigma) in deionized water. The treatment rapidly reduced the viscosity of the supernatant, indicating hyaluronan was a major component of the viscous media.

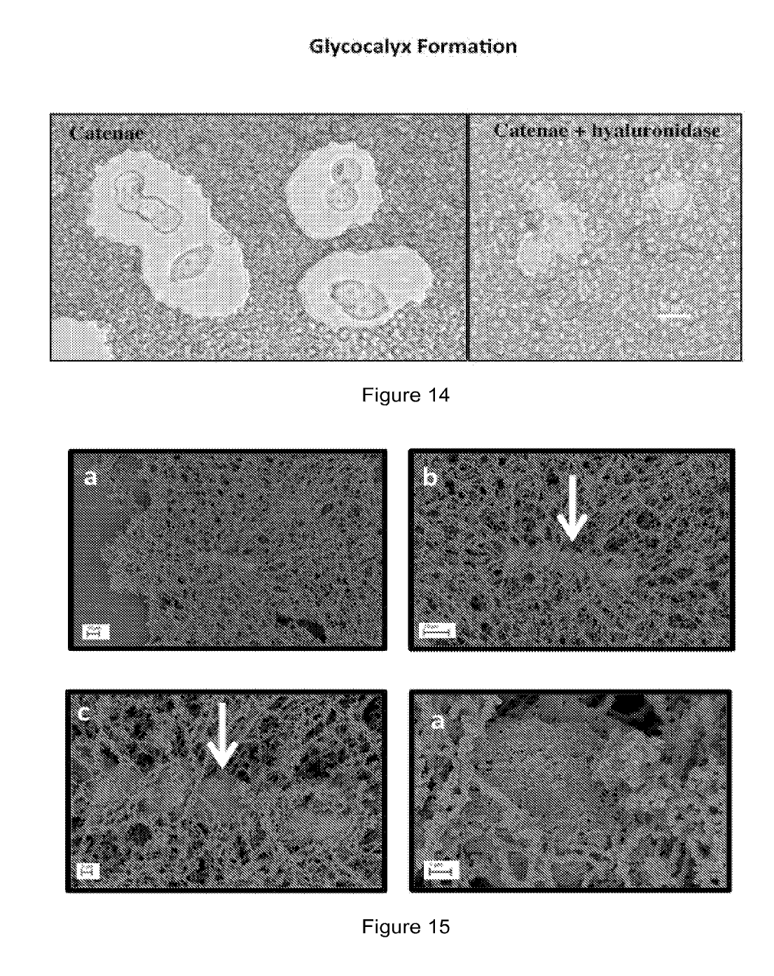

To visualize the glycocalyx surrounding a catena, a particle exclusion experiment was conducted using red blood cells (RBCs). Catenae were mechanically dissociated by pipetting or by brief incubation with hyaluronidase as before. RBCs from human peripheral blood were added and the mixture was incubated overnight in culture media. The cells were observed under the light microscope for the presence of a glycocalyx separating catena cells from the RBCs. Mechanically-dissociated catenae mixed with RBCs had a glycocalyx coat extending up to 25 μm from the cell surface (FIG. 14, left panel), preventing direct catena-RBC cellular contact, whereas hyaluronidase-treated catena completely lacked a glycocalyx, allowing RBC-catena interaction (FIG. 14, right panel).

Because glycocalyx formation correlated with mesenchymal to amoeboid transition, the maintenance of glycocalyx integrity may be necessary for symmetric expansion of ovarian CSCs as catenae (and other serosal CSCs). For example, the glycocalyx may prevent integrin interactions with extracellular matrix, suggesting that removal of the glycocalyx should expose cell surface proteins and allow interactions with extracellular matrix or other attachment surfaces.

To investigate the how catena cells grow upon disruption of the glycocalyx, catenae were dissociated to single cells with hyaluronidase treatment and plated in tissue culture treated flasks with or without 10% hyaluronidase enzyme solution (10 mg/ml) to prevent the formation of glycocalyx. In parallel, catenae were dissociated mechanically and plated in the absence of hyaluronidase.

Mechanically-dissociated catenae remained in suspension where they proliferated rapidly to form free-floating chains of cell. Catenae dissociated to single cells with a brief treatment of hyaluronidase and plated in the absence of hyaluronidase enzyme no longer formed free floating chains but rather proliferated as irregular aggregates in suspension. In contrast, continuously hyaluronidase-treated cells attached to tissue culture plates and formed epithelial and mesenchymal monolayers. The results suggest that without a protective coat, ovarian CSCs are able to interact with attachment surfaces and respond to downstream differentiation stimuli.

The presence of different types of monolayers cells in these cultures validated the multilineage differentiation potential of ovarian CSCs from catenae. Epithelial monolayers were less frequently observed than mesenchymal cells indicating that more differentiation signals are needed to generate epithelial cancer cells than for mesenchymal cancer cells.

### Example 17

### Catena Glycocalyx Composition

### 1. Low Molecular Weight Hyaluronan-Collagen Complex

Catena glycocalyx have two major components, i.e., hyaluronan and collagen, which interact and form a stable complex. Western blot analysis showed a low molecular weight complex of collagen and hyaluronan (less than 20 kDa), detectable by anti-COL1A2 antibody. Briefly, the supernatant fraction of catena cell cultures was separated from the cells by centrifugation. The supernatant was run in an SDS-PAGE gel and blotted with the anti-COL1A2 antibody. This complex was sensitive to hyaluronidase treatment but was not affected by collagenase type 1, 2 or 4 treatment. This hyaluronan-collagen complex could be important for the formation of catena glycocalyx and drug resistance or metastatic potential conferred to catena cells by the glycocalyx.

2. Expression of Extracellular Matrix Genes Catenae

The extracellular matrix of catena is isolated and analyzed for proteins present in catena glycocalyx as validated by deep sequencing and mass spectrometry of the secretome of catena cells.

Two important components of the extracellular matrix, elastin and fibronectin are not expressed by catenae. Laminin and collagen are major component of the catena glycocalyx along with hyaluronan. Hyaluronan and proteoglycans are linked and stabilized by HAPLN1 (hyaluronan proteoglycan link protein 1), HABP1 (hyaluronan binding protein 1) and HABP4 (hyaluronan binding protein 1) proteins. Each component of the glycocalyx is essential for the integrity of the coat and any changes in the composition effects the cell morphology and associated characteristics. When catena cells roll-up and form tumor spheroids, LUM (lumican), DCN (decorin) and JAM2 (junctional adhesion molecule 2), COL6A1 (collagen, type VI, alpha 1), COL6A2 (collagen, type VI, alpha 2), SGCG (sarcoglycan, gamma) genes are upregulated but HAPLN1, VCAN (versican) and GPC3 (glypican 3) genes are downregulated. Therefore, the glycocalyx of the spheroids are different than catena glycocalyx.

Table 4 lists extracellular matrix proteins that are upregulated and present in catenae (left column) and proteins that are downregulated in catenae (right column) The catena secretome fraction was analyzed for the presence or absence of these gene products and none of the down regulated genes were detected in that fraction.

TABLE 4
Extracellular Matrix Proteins In Catenae

| Protein in | Downregulated |
|---|---|
| Glycocalyx | Gene in Catena |
| VCAN | ELN |
| NID1 | FN1 |
| NID2 | ACAN |
| MGP | DCN |
| LAMA5 | LUM |
| LAMB2 | TNXB |
| LAMC1 | AGRN |
| COL1A1 | |
| COL1A2 | |
| COL3A1 | |
| COL4A5 | |
| COL4A3BP | |
| COL5A2 | |
| COL6A3 | |
| COL6A1 | |
| HABP1/C1QBP | |
| HABP4 | |
| HAPLN1 | |

### Example 18

### Clonogenicity of Hyaluronidase-Treated Catenae

Catena cells were dissociated with hyaluronidase, allowed to attach to tissue culture plates and grown in the presence hyaluronidase for 7 days. Under these conditions, cells remained attached to tissue culture plates. The cells were harvested and subjected to an in vitro clonogenicity assay in the presence and absence of hyaluronidase. In parallel, mechanically-dissociated catena were subjected to the in vitro clonogenicity assay in the presence and absence of hyaluronidase.

Attached cells proliferated significantly slower than free-floating catenae and formed predominantly attached colonies with only a few cells “piling up” on mesenchymal and epithelial monolayers. The colony size was further reduced if hyaluronidase enzyme was included in the clonogenic assay. These results show that glycocalyx composed of hyaluronan is involved in maintaining the free-floating chain morphology and cancer stem cell characteristics of catenae.

### Example 19

### Combination Drug Screening

The glycocalyx around the catenae confers resistance to some therapeutic agents such as paclitaxel, fludelone and 9,10-dEpoB but not to others such as deguelin and bortezomib (See, Example 15). Since hyaluronan and collagen are major components of the catena glycocalyx, we tested whether treatment of catena cells with hyaluronidase and/or collagenase altered the drug resistance of catena cells.

### 1. PEGylation

Hyaluronidase and collagenase have short half lives in vivo and modification of these enzymes by attachment of polyethylene glycol (PEG; the process being PEGylation) has been shown to increase the stability of enzymes from minutes to several hours. To PEGylate these enzymes, alpha-methoxy-omega-carboxylic acid succinimidyl ester polyethylend glycol (PEG MW 20,000) (MeO-PEG-NHS) was used by mixing 100 mg MeO-PEG-NHS with 0.5 mL 10 mg/mL bovine testis hyaluronidase (25000 U/mL) and 15 ml PBS. The mixture was incubated at 4° for 48 hrs on a rotator. For PEGylation of collagenase, 0.5 mL of 10 mg/mL collagenase 1 (2500 U/mL) was substituted for the hyaluronidase.

The PEGylated and non-PEGylated samples, reduced and non-reduced, were run by protein gel electrophoresis and stained with Coomassie blue. The expected increases in band size were observed, including the addition of multiple PEG moieties.

To examine whether PEGylation inhibited enzymatic activity, catenae were treated with PEGylated or non-PEGylated hyaluronidase [as described above]. Both treatments caused aggregation of catena cells. Addition of collagenase 1 to catena cultures does not affect the morphology of those cells, and similarly, addition of PEGylated collagenase 1 did not have any effect on catena morphology.

### 2. Drug Screening

Twenty-five catena cells were seeded into 384-well plates. After 5 days, cells were treated with either PEGylated hyaluronidase, PEGylated collagenase or both for 10 minutes at 37° C. Without removing the enzymes, paclitaxel was added over a series of dilutions, followed by alamarBlue addition on day 9 with absorbance measured two days later. The IC50 for paclitaxel alone was unchanged in the presence of PEGylated collagenase. Treatment of the cultures with PEGylated hyaluronidase prior to adding paclitaxel decreased the IC50 by 2.5 fold and treating with the combination PEGylated enzymes, decreased the IC50 by 16 fold for paclitaxel, a value comparable to that obtained when paclitaxel was added to plates 1 day after cell seeding, i.e., when the catena cells lacked any substantial amount of glycocalyx.

### Example 20

### Effects of Basement Membrane Matrix on Catena Morphology

Ovcar3-GTL catenae were dissociated to single cells by mechanical dissociation or by hyaluronidase treatment and cultured on basement membrane matrix (Matrigel) coated plates. A similar set of cultures were grown in the presence of 1 mM 4-methylumbelliferone (4-MU) and 50 μM Y27632, the former being an hyaluronan synthase 2 (HAS2) inhibitor and the latter being a Rho-ROCK inhibitor. The cultures were imaged after 4 days.

Without hyaluronidase treatment, catenae retained their glycocalyx, did not interact with the extracellular matrix components and formed free-floating chains of cells as expected. Cells treated with only hyaluronidase attached to extracellular matrix and grew as attached irregular aggregates. When hyaluronidase-pretreated cells were grown with 4-MU and Y27632, the cultures did not become viscous and the attached cells formed filopodial extensions. Likewise, cultures of mechanically-dissociated cells grown in the presence of 4-MU and Y27632 did not become viscous; rather, the cells attached to the plates and formed filopodial extensions.

The small GTPase, Rho, and its target protein, Rho-associated coiled-coil-forming protein kinase (ROCK), have been recognized as regulators of mesenchymal to amoeboid transition (MAT). During MAT, the up regulation of Rho-ROCK activity helps to generate sufficient actomyosin forces to allow tumor cells to deform collagen fibers and push through the extracellular matrix [Wyckoff, 2006]. Inhibition of Rho-ROCK activity in catena cultures caused cell attachment and induced the formation of filopodial extensions indicating a reversion to mesenchymal morphology.

### Example 21

### Catenae Morphology under SEM and TEM

The initial attempts to visualize the glycocalyx coat of catenae by electron microscopy using standard methodology were unsuccessful. Hence, a new protocol was developed to visualize the pericellular structures of catena cells by scanning electron microscopy (SEM).

Briefly, catenae were grown in M5-FCS media. Aliquots of catena cultures were placed on poly-L-lysine-coated plastic coverslips and cells were allowed to adhere for 1 hr at room temperature in a moist chamber. Without washing off the suspension of cells, the fixatives (2.5% glutaraldehyde/2% paraformaldehyde in 0.75 M cacodylate buffer) were added directly onto the cover slips and incubated at room temperature for 1 hr in a moist chamber. In this technique, the negatively charged extracellular viscous coat of the cells attached to the positively charged surface. Cells were trapped in the extensive extracellular meshwork of hyaluronan, proteoglycans and collagens. By adding the fixative directly on to the attached cell-glycocalyx mixture before the washing step, the structure of cells and extracellular coat was preserved. When used, stains were included with the fixative; Alcian Blue (AB) to stain sugars (in this case hyaluronan chains) and cetylpyridinium chloride (CPC) to stain proteoglycans. This combination of dyes helped to visualize all components of the glycocalyx at the same time.

After the fixative step, the preparations were rinsed in cacodylate buffer and dehydrated in a graded series of ethanol solutions from 50%, 75%, 95% through absolute alcohol. The samples were critical point dried in a Denton Critical Point Dryer Model JCP-1 and sputter coated with gold/palladium in a Denton Vacuum Desk 1V sputtering system. The samples were photographed using a Zeiss Field Emission Electronmicroscope Supra 25.

Intracellular structures and organelles were visualized by TEM.

The present method succeeded in establishing a protocol to adhere catena cells onto coverslips while retaining their pericellular coat and identifying specialized structures associated with the catenae.

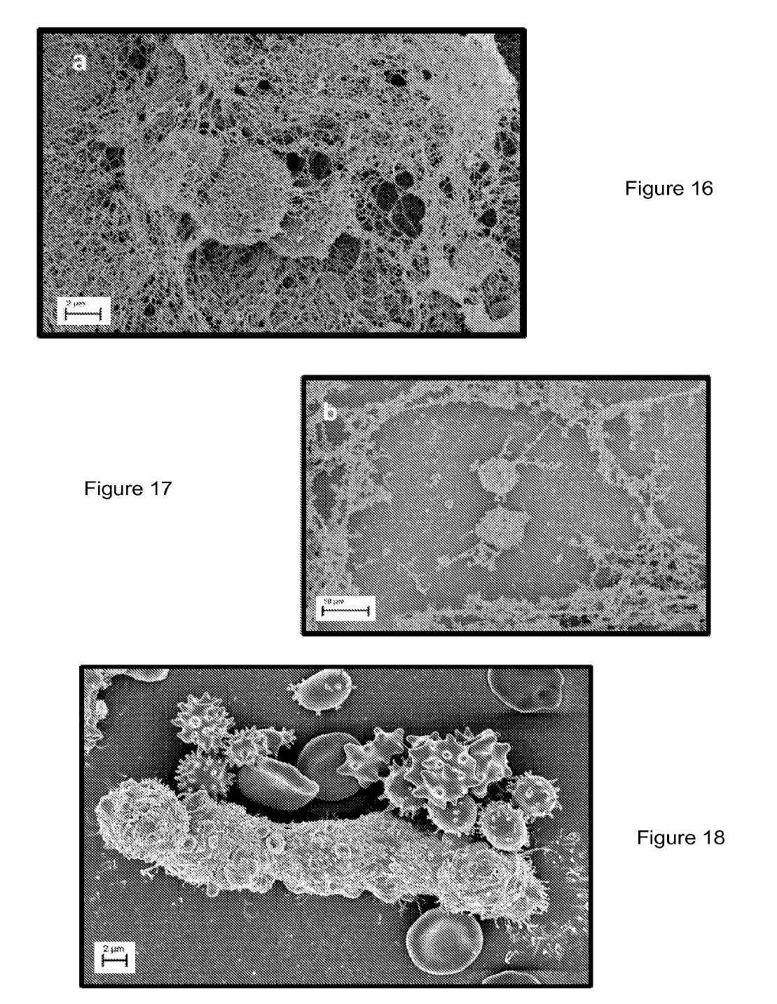

FIG. 15 shows a series of SEM images at different magnifications of a catena displaying the extensive glycocalyx after AB and CPC staining FIG. 16 presents an enlarged SEM image of a catena and glycocalyx stained only with AB, showing the hyaluronan coat over the cells, that hyaluronic acid concentrates at various points and the web like nature of the hyaluronan coat.

Catenae were treated with hyaluronidase to remove the glycocalyx coat and viewed by SEM with AB and CPC staining As shown in FIG. 17, remnants of the glycocalyx are visible.

FIG. 18 is an SEM image of an unstained catena after treatment with hyaluronidase to remove the glycocalyx coat. The other cells present in the sample are RBCs (including smooth and spiky RBCs). Note the unusual surface of the catena.

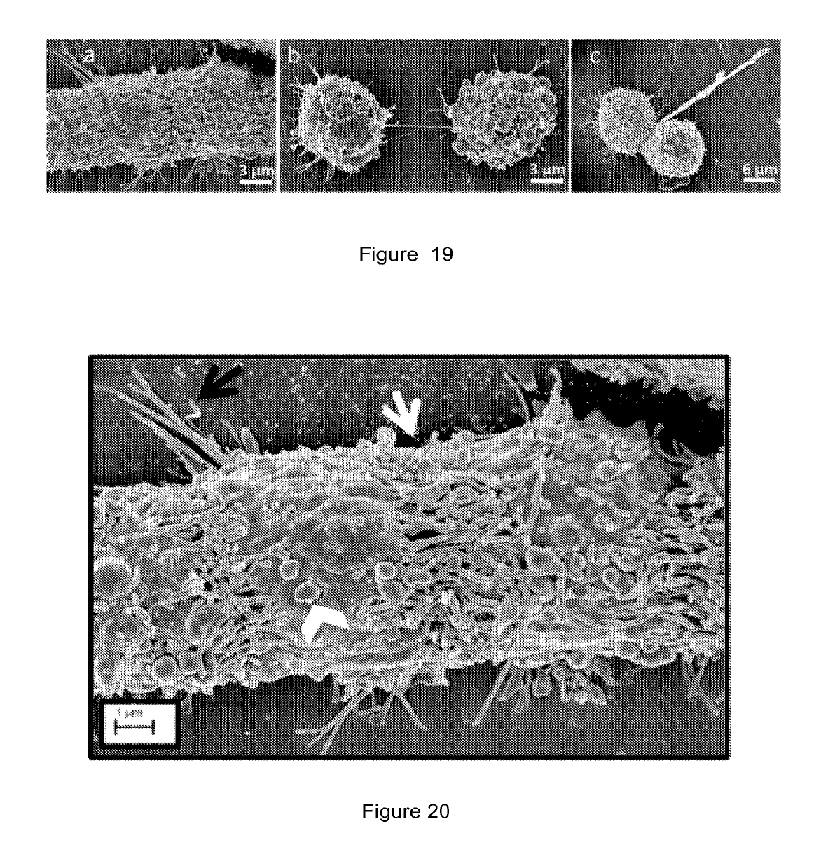

Catena structures include microvilli, surface blebs, pseudopodia and nanotubes, volcanoes and craters as visible in the SEM images shown in FIGS. 19-21. FIG. 19(a) shows an SEM micrograph of a unstained catena with extensive microvilli connections between the cells. In FIG. 19(b), two catena cells are connected by a nanotube and the cells appear to attach to the surface via microvilli (invadopodia). Large plasma membrane blebs are also visible on these cells. FIG. 19(c) shows unstained catena cells with a long pseudopodium (20-30 μm) extending beyond the 10-15 μm space occupied by the hyaluronan glycocalyx.

In light micrographs (not shown), a catena cell stained with a cell membrane lipophilic dye showed punctuate staining and showed solid, conintuous staining with an antibody to hyaluronan. Surface blebs breaking the surface and protruding through the hyaluronan staining were also observed. Pseudopodia extending through the hyaluronan glycocalyx can be visualized by staining with cell membrane lipophilic dye and have been observed to fold over to form lasso-shaped structures.

Various structures on the catena surface are shown in enlarged form in FIG. 20 which is an enlarged version of the photograph in FIG. 19(a) and has arrows highlighting microvilli, pseudopodia and surface blebs. An SEM image of catena microvilli showed their segmented nature and many SEM images showed extensive surface blebbing present on catena cells.

With TEM one can visualize the structures in a plane through a cell. Such images showed blebs continuous with the cellular membrane of the catena cell but also adjacent to the cell. The blebs appeared homogenous in content and lacked large cellular organelles. Further the catena cell images showed undifferentiated cell morphology, indicative of its stemness, i.e., a high nucleus/cytoplasm ratio, and microvilli forming continuous boundaries at the surface of the cells.

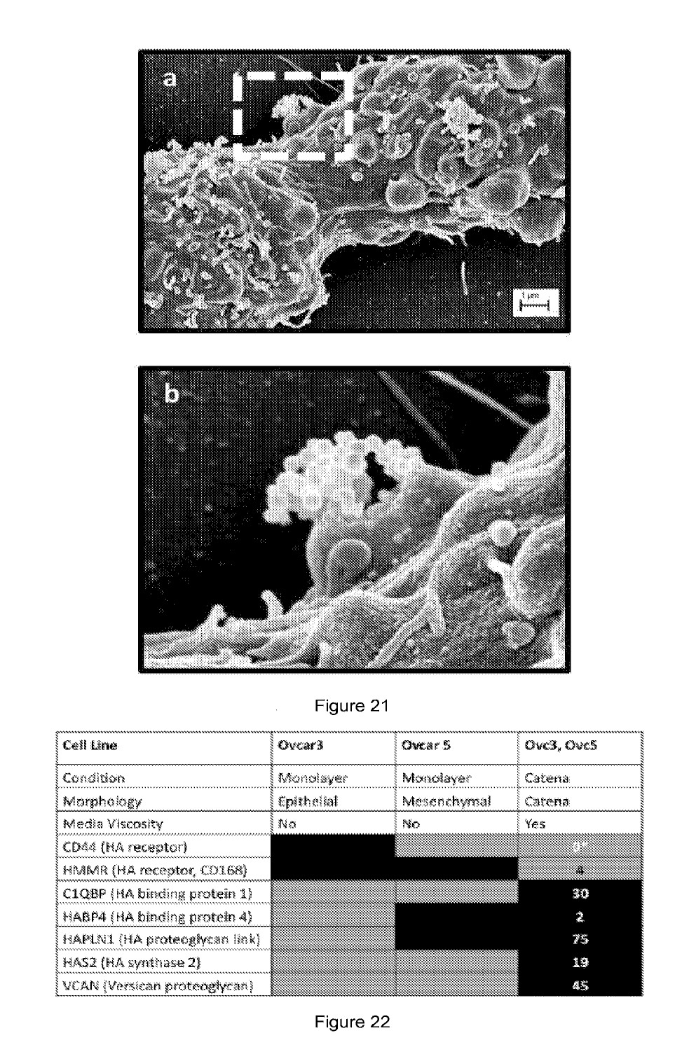

The appearance of volcano-like structures on the catena cells was an unusual finding. The SEM image in FIG. 21 shows a side view of (a) an erupting “volcano” on the catena surface and (b) an enlargement of the volcano showing the release of particles from the crater of the volcano and which appear to be exosomes. In a top down view of a cell, an apparent surface crater was present which could be the fusion of an internal bleb with the outer cell membrane. This crater had a discreet boundary like appearance around its rim and small, vesicular-like particles inside the crater. Surface blebs were also observed on this cell.

### Example 22

### Gene Expression in Catenae

For gene expression studies, RNA was extracted using TRIzol® Reagent (Invitrogen). Gene expression was determined using Affymetrix U133 plus 2.0 arrays with 3 biological replicates per sample. Data were analyzed using Genespring GX Software (Agilent). Ovcar3, Ovcar5 and A2780 spheroids, catenae and monolayers and SV-40 immortalized normal ovarian epithelial monolayers (NOE; T-80 cells) were analyzed. Gene annotations can be found at www.ncbi.nlm.nih.gov/gene.

A total of 2121 genes were differentially expressed between Ovcar3-GTL catenae and Ovcar3GL monolayer. Of these genes, 1125 genes were upregulated and 996 genes were downregulated in catena compared to monolayers. A total of 378 genes were differentially expressed between the NOE T-80 cells and the Ovcar3-GL monolayers. Of these, 101 genes were upregulated and 277 genes were down-regulated in Ovcar3-GTL monolayers compared to T-80 cells.

Gene expression in Ovcar3 and Ovcar5 catenae was compared to that in Ovcar5 mesenchymal monolayers. The combined transcriptome analysis identified 26 upregulated genes and 69 downregulated genes in this mesenchymal to amoeboid/catena transition, i.e., differentially expressed in catenae/CSCs (Table 5). The most upregulated gene was hyaluronan synthase (HAS2). The second most expressed gene was PDGFRA indicating a significant role for the PDGF pathway in catenae/CSCs.

TABLE 5
Differentially Expressed Genes in Catenae

| | |
|---|---|
| Up Regulated | Down Regulated |
| Genes | Genes |

| | | | |
|---|---|---|---|
| HAS2 | LUM | C10orf75 | TMEM22 |
| PDGFRA | DCN | ERCC1 | GSG1 |
| HAPLN1 | COLEC12 | GPC4 | CCR1 |
| MGST1 | EGR3 | GLUL | KBTBD2 |
| S100A4 | EGR1 | LEPR | B4GALT6 |
| FAS | LIPG | SDK2 | DOCK8 |
| TP53I3 | GPR137B | TGIF1 | DCLRE1C |
| NSBP1 | KERA | KIAA0746 | RBM24 |
| CLIC4 | GPR126 | TUBB2B | TFEC |
| GLRX | GABBR2 | NPNT | IER5L |
| RGS2 | EGR4 | WNK4 | TBX1 |
| DDB2 | AREG | TAGLN | HTRA1 |
| WTAP | HTR2B | AFAP1L1 | TRIB3 |
| MAP2K6 | UPP1 | RAB31 | SOX8 |
| RPS27L | SLC35D3 | FNDC1 | CAMK2B |
| FDXR | IER5L | SERPINE1 | JMJD3 |
| NTS | EMP1 | ZNF804A | ZNF398 |
| SPATA18 | TAGLN | JAG1 | GRHL1 |
| ARG2 | WWTR1 | BCAN | RAB11FIP4 |
| COL4A3BP | C9orf3 | TFEC | PABPC4L |
| RNF145 | AKAP12 | ARHGAP5 | ZSWIM6 |
| ANAPC7 | HES1 | EREG | SLC30A7 |
| PHF14 | LONRF2 | MIDN | JUNB |
| MAB21L2 | | | |
| LOC643401 | | | |
| C6orf54 | | | |

HAS2 is one of three synthases responsible for production of the glycosylaminoglycan hyaluronan (HA). A number of genes for hyaluronan-binding proteins were also upregulated in catenae when compared to the Ovcar3 epithelial monolayer, including HA-binding “link” proteins C1QBP, HABP4 and HAPLN1 and the proteoglycan Versican/VCAN (FIG. 22). The HA receptors CD44 and HMMR were differentially downregulated in catenae. HAS2 was not expressed at significant levels in either the mesenchymal or epithelial monolayers.

The stem cell-associated genes Lin-28, Bmi-1, RBPMS and ZFX were all expressed in catenae. RBPMS is expressed in hematopoietic stem cells, embryonic stem cells, neural stem cells, leukemia stem cells, leukemia and in germ cell tumors. ZFX zinc finger transcriptional regulator, which has been shown to control self-renewal of embryonic and hematopoietic stem cells, is also upregulated in catenae and spheroids when compared to epithelial monolayers.

Telomerase reverse transcriptase component hTERT, TERF1, TERF2 and Tankyrase are part of the telomerase pathway that is upregulated in cancer and in Ovcar3-GTL monolayer tumor cells relative to normal ovarian epithelium. However, catenae have even higher expression of these genes than do spheroids or monolayers indicating that anti-telomerase therapy could be efficient for targeting the CSC in ovarian cancer.

Additional gene expression data relating to the surfaceome is described in Example 27.

### Example 23

### Expression of Upregulated Catena-Specific Genes in Other Tissues

Amazonia! (Le Carrour et al., 2010) provides a web atlas of publically available human transcriptome data which can be queried to determine the tissue expression pattern of a specific gene. The upregulated catena genes from Table 5 were analyzed in this manner. Those genes found to have restricted tissue expression patterns, and the tissue or cell type of that expression are set out in Table 6. The remaining upregulated catena genes did not show a tissue-restricted expression pattern against the Amazonia! data (indicated by no tissue or cell type in Table 6). Many of the upregulated catena genes, were found to be expressed in human embryonic stem cells (hESCs) and in human, induced pluripotent stem cells (hIPSCs) and not in normal adult tissues and cell types (in which group the Amazonia! database includes tissue-specific stem cells). The genes found to be expressed in hESCs include HAS2, HAPLN1, NTS, and LOC643401. Genes that were downregulated in catenae had broad expression patterns in normal adult tissues and cell types without expression in embryonic stem cells.

TABLE 6
Tissue Gene Expression of Unregulated Catena Genes

| Up Regulated | Tissue or Cell |
|---|---|
| Catena Gene | Expression |
| HAS2 | hESC, hIPSC |
| PDGFRA | |
| HAPLN1 | hESC, hIPSC |
| MGST1 | |
| S100A4 | |
| FAS | |
| TP53I3 | |
| NSBP1 | |
| CLIC4 | |
| GLRX | |
| RGS2 | OOCYTE |
| DDB2 | |
| WTAP | OOCYTE |
| MAP2K6 | |
| RPS27L | OOCYTE |
| FDXR | ADRENAL GLAND |
| NTS | hESC, hIPSC |
| SPATA18 | TESTIS |
| ARG2 | OOCYTE |
| COL4A3BP | |
| RNF145 | |
| ANAPC7 | |
| PHF14 | OOCYTE |
| MAB21L2 | COLON, INTESTINE |
| LOC643401 | hESC, hIPSC |
| C6orf54 | |

### Example 24

### Analysis of Gene Expression Profiles in the Cancer Geneome Atlas

The gene expression profiles of 366 advanced stage ovarian cancer patients and 10 normal ovary samples were available through The Cancer Genome Atlas (TCGA; http://tcga.cancer.gov) and analyzed for expression of the upregulated and downregulated catena genes in Table 5. These gene expression profiles represent microarray analysis of mRNA in RNA isolated from the tumor samples expression data. The catena-specific genes were then queried against these tumor gene expression profiles in the TCGA database.

This analysis enabled identification of clusters of patients according to particular sets of expressed catena genes and begins to define one type of catena gene signature for ovarian cancer patients. For example, the 9 upregulated catena genes shown as LIST 1 in Table 7, which includes COL1A2 that had also been identified by mass spectrometry as secreted at higher amounts in catenae relative to Ovcar5 and A2780 mesenchymal cells, defined a group (or cluster) of 83 patients that co-expressed high levels of at least 6 out of 9 of these genes and suggests that this patient cohort has a higher proportion of catena cells (i.e., ovarian cancer stem cells). Additional catena-specific genes that were expressed in this cluster of patients are shown as LIST 2 in Table 7. LIST 3 in Table 7 identifies catena genes that were expressed in both the cluster patient samples and in normal ovary samples. The genes in LIST 4 are ovarian cancer marker genes that are significantly downregulated in catena cells when compared to differentiated tumors.

TABLE 7
TCGA Gene Expression and Cluster Analysis

| | Cluster- | Other Catena | Catena Genes in | Cancer Markers |
|---|---|---|---|---|
| | Defining | Genes in | Cluster and Normal | Downregulated in |
| | Genes | Cluster | Ovary | Catena |
| | LIST 1 | LIST 2 | LIST 3 | LIST 4 |
| 1 | COL1A2 | TWIST1 | VIM | CD44 |
| 2 | HAS2 | COL3A1 | MEOX2 | CDH1 |
| 3 | HAPLN1 | FGF18 | PDGFC | CLDN3 |
| 4 | PDGFRA | THY1 | JAM3 | CLDN4 |
| 5 | S100A4 | TJP1 | RGS2 | CTAG1A |
| 6 | FAS | RGS16 | HGF | EPCAM |
| 7 | CLIC4 | LUM | MARCKS | FOLR1 |
| 8 | GLRX | SERPINE1 | FAS | MSLN |
| 9 | RGS2 | NTS | ZEB1 | MUC16 |
| 10 | | MAB21L2 | DCN | PROM1 |
| 11 | | EMP1 | TAGLN | SLC34A2 |
| 12 | | FN1 | LHFP | WT1 |
| 13 | | SLC12A8 | SNAI2 | |
| 14 | | COL4A1 | RAB31 | |
| 15 | | ZFHX4 | FZD1 | |
| 16 | | COL12A1 | PDGFRB | |
| 17 | | PDGFB | COLEC12 | |
| 18 | | COL4A2 | PDGFA | |
| 19 | | MAFB | PDGFD | |
| 20 | | SNAI1 | TGFBR2 | |
| 21 | | FNDC1 | CD36 | |
| 22 | | RUNX1 | ARID5B | |
| 23 | | ODZ3 | | |

### Example 25

### Differential miRNA Expression in Catenae

For miRNA analysis, RNA was extracted using TRIzol® Reagent (Invitrogen). miRNA expression was determined by using Agilent Human microRNA Array V1.0, which contains probes against all human miRNAs (˜500) on the Sanger mirBase release 9.1 (February 2007). Two biological replicates per sample were analyzed for miRNA expression. Data were analyzed using Genespring GX Software (Agilent).

The results for the up regulated and downregulated miRNA in catenae compared to Ovcar3 monolayers are summarized in Table 8.

26 miRNAs were downregulated in catenae compared to Ovcar3 monolayers. These included the let-7 family miRNAs that are regulated by Lin28 and Lin28B. Lin28 mRNA and protein were significantly upregulated in catenae compared to normal ovarian epithelium and Ovcar3 epithelial monolayer cells. It was the most upregulated gene in catenae when compared to spheres. LIN28B, a close homolog of LIN28, was significantly and differentially upregulated in catena vs Ovcar3 epithelium.

All five members of miR-200 family (miR-141, miR-200a, miR-200b, miR-200c and miR-429) were significantly down-regulated in the catenae compared to Ovcar3 epithelial monolayers. Inhibition of the miR-200 family is reported to be sufficient to induce EMT and in analysis of the NCI panel of 60 tumor cell lines the miR200 family was expressed in epithelial ovarian cancer cell lines but was lost in mesenchymal ovarian cell lines (Gregory et al. 2008; Park et al. 2008). The data in this study is in concordance with other reports of significant down-regulation of miR-200b, miR-200c, let-7b, let-7c, let-7d, and let-7e in various tumor cell types that have undergone EMT associated with elongated fibroblastoid morphology, lower expression of E-cadherin, and higher expression of ZEB1 (Park et al. 2008).

Further, hsa-miR-23b and hsa-miR-27b were significantly downregulated in catena compared to mesenchymal monolayers. Target prediction analysis showed that HAS2 is a target of hsa-miR-23b. Further, hyaluronan proteoglycan link protein-1 (HAPLN1) and platelet-derived growth factor receptor alpha (PDGFRA, also written as PDGRα) are both targets of hsa-miR-27b. Hence, the results show a significant correlation between the three most upregulated genes in catena cells (HAS2, HAPLN1, PDGFRA) and downregulation of hsa-miR-23b and hsa-miR-27b.

TABLE 8
Human miRNA Gene Expression Changes in Catena
Cells Relative to Epithelial Monolayer Cells

| | human miRNA | Expression in Catena |
|---|---|---|
| 1 | 363 | upregulated |
| 2 | 630 | upregulated |
| 3 | 18b | upregulated |
| 4 | 214 | upregulated |
| 5 | 367 | upregulated |
| 6 | 302a | upregulated |
| 7 | 302a* | upregulated |
| 8 | 302b | upregulated |
| 9 | 302c | upregulated |
| 10 | 138 | upregulated |
| 11 | 422a | upregulated |
| 12 | 493-3p | upregulated |
| 13 | Let-7a | downregulated |
| 14 | Let-7c | downregulated |
| 15 | Let-7d | downregulated |
| 16 | Let-7f | downregulated |
| 17 | Let-7g | downregulated |
| 18 | Let-7i | downregulated |
| 19 | 125b | downregulated |
| 20 | 141 | downregulated |
| 21 | 200a | downregulated |
| 22 | 200b | downregulated |
| 23 | 200c | downregulated |
| 24 | 30a-5p | downregulated |
| 25 | 31 | downregulated |
| 26 | 429 | downregulated |
| 27 | 517c | downregulated |
| 28 | 521 | downregulated |
| 29 | 23b | downregulated |
| 30 | 27b | downregulated |
| 31 | 128a | downregulated |
| 32 | 145 | downregulated |

### Example 26

### RTK Phosphorylation in Epithelial, Mesenchymal and Catena Cells

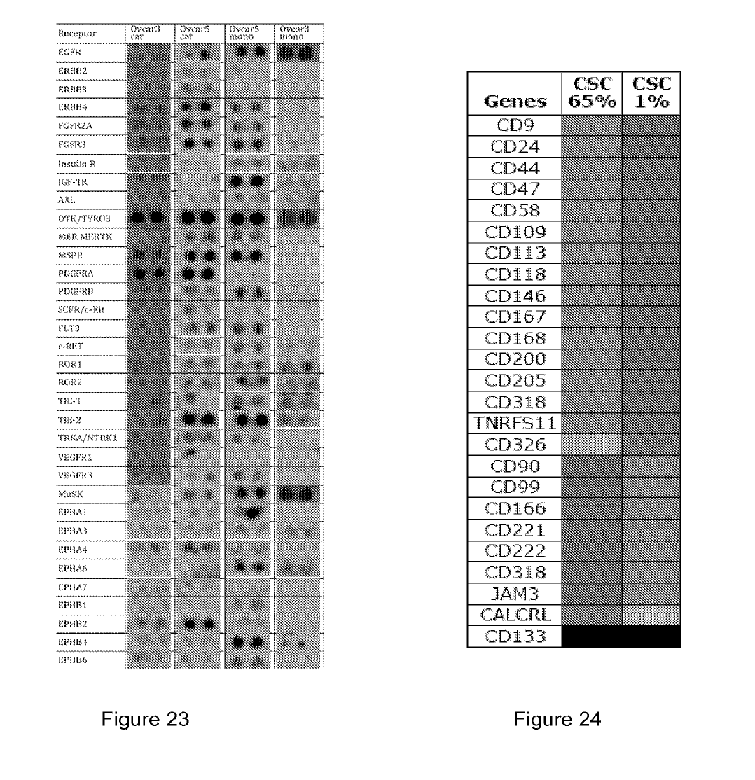

The Human Phospho-RTK Array Kit (R&D Systems) was used according to manufacturer's directions to determine the phosphorylation status of a panel of 42 receptor tyrosine kinase (RTK) proteins in Ovcar3-GTL and Ovcar5-GL catenae or monolayers. In the assay, nitrocellulose membrane dot arrays have capture antibodies against the extracellular domain of each RTK, cell lysates are incubated with the arrays, and a pan- phosphotyrosine antibody conjugated to HRP is used to visualize the activated (phosphorylated) proteins using chemiluminescence. For these cells were grown in the presence of 10% FCS. The list of 42 RTK proteins is provided in Table 9 and the results for 34 of these proteins are shown in FIG. 23.

The EGFR and DTK (AXL receptor family) were phosphorylated in Ovcar3 epithelial monolayers. In contrast, in mesenchymal Ovcar5 monolayers multiple signaling pathways were activated (22/44 receptors tested) (FIG. 23) including PDGFRβ, EGFR, ERBB4, FGFR2, FGFR3, Insulin-R, IGF1R, DTK/TYRO3, MER/MERTK, MSPR/RON, Flt-3, c-rRET, ROR1, ROR2, Tie-1, Tie-2, TrkA/NTRK1, VEGFR3, EphA1, EphA3, EphA4, EphA7, EphB2, EphB4, and EphB6. Catenae cells derived from Ovcar3 and Ovcar5 had at least qualitatively similar phospho-RTK profiles and to Ovcar5 mesenchymal monolayers, with 17/22 phosphorylated receptors in Ovcar5 monolayers also active in both types of catenae. Nevertheless, there were differences in the degree of phosphorylation of specific receptors between the two sources of catenae and between these and the mesenchymal monolayer. For example, phosphorylation of PDGFRα distinguished the amoeboid catena cells from Ovcar5 mesenchymal monolayers. The data supports the concept that multiple RTK phosphorylation is linked to epithelial-mesenchymal transition.

TABLE 9
RTK Proteins Content of Human Phospho-RTK Array Kit

| Receptor Family | RTK/Control |
|---|---|
| EGF R | EGF R |
| EGF R | ErbB2 |
| EGF R | ErbB3 |
| EGF R | ErbB4 |
| FGF R | FGF R1 |
| FGF R | FGF R2. |
| FGF R | FGF R3 |
| FGF R | FGF R4 |
| Insulin R | Insulin R |
| Insulin R | IGF-I R |
| Axl | Axl |
| Axl | Dtk |
| Axl | Mer |
| HGF R | HGF R |
| HGF R | MSP R |
| PDGF R | PDGF R. |
| PDGF R | PDGF R. |
| PDGF R | SCF R |
| PDGF R | Flt-3 |
| PDGF R | M-CSF R |
| RET | c-Ret |
| ROR | ROR1 |
| ROR | ROR2 |
| Tie | Tie-1 |
| Tie | Tie-2 |
| NGF R | TrkA |
| NGF R | TrkB |
| NGF R | TrkC |
| VEGF R | VEGF R1 |
| VEGF R | VEGF R2 |
| VEGF R | VEGF R3 |
| MuSK | MuSK |
| Eph R | EphA1 |
| Eph R | EphA2 |
| Eph R | EphA3 |
| Eph R | EphA4 |
| Eph R | EphA6 |
| Eph R | EphA7 |
| Eph R | EphB1 |
| Eph R | EphB2 |
| Eph R | EphB4 |
| Eph R | EphB6 |

### Example 27

### Catena Surface Phenotype

### 1. FACS Surfacesome Analysis

Multi-parameter flow cytometric evaluation was undertaken with collagenase IV-dissociated Ovcar3-GTL catenae and spheroids or with trypsin-dissociated monolayers. Primary ovarian cancer ascites samples were dissociated by dispase treatment followed by lymphoid and hematopoietic cell depletion using CD45 +-magnetic bead removal. Cells were stained in a total volume of 100 μL containing the appropriate antibodies and MACS-buffer.

For analysis the following antibodies were used: CD45-APC-Cy7 (clone 2D1), CD34-APC (clone 8G12), CD44-PE (clone G44-26), CD49f-PE (clone GoH3) and CD90-APC (clone SE10) (all BD Pharmingen); CD133-APC (clone AC133), CD133-PE (clone 293C3) and CD326-FITC, -PE, -APC (clone HEA-125) (all Miltenyi Biotec) and CXCR4-PE (clone 12G5) (R&D Systems, Inc) as well as the antibodies listed in Table 10. For dead cell exclusion DAPI (Invitrogen) was added. All flow cytometric analyses were performed on a FACS Calibur (Becton-Dickinson) acquiring 1-25×10⁴ events per sample using a MoFlo Cell Sorter. Data were analyzed using FlowJo 7.2.2 software (Tree Star, Inc).

Catenae were >95% positive for CD49f (alpha6 integrin) and CD90 d(Thy-1), negative for CD34 and CD133 (with 2 different antibodies).

Catenae derived from Ovcar3-GTL and Ovcar5-GL cell lines had very similar phenotypes. As observed in mesenchymal monolayers, most of the surface antigens including Epcam (CD326) and Muc16 (CA125) were absent on Catenae. GM2 stained 98% of Ovcar5-GL catenae and 74% of Ovcar3-GTL catenae.

The only significant difference between catenae and mesenchymal cells was the expression of Mucin 1 (CA15-3). Mucin 1 stained 65% of mesenchymal monolayers cells but only 6% of Ovcar3-GTL catenae and 75% of Ovcar5-GL catenae were positive of Mucin 1. Mucin 1 also stained 75% of Ovcar3 epithelial monolayer cells. The surfaceome data is summarized in Table 10.

### 2. CD Gene Expression Analysis

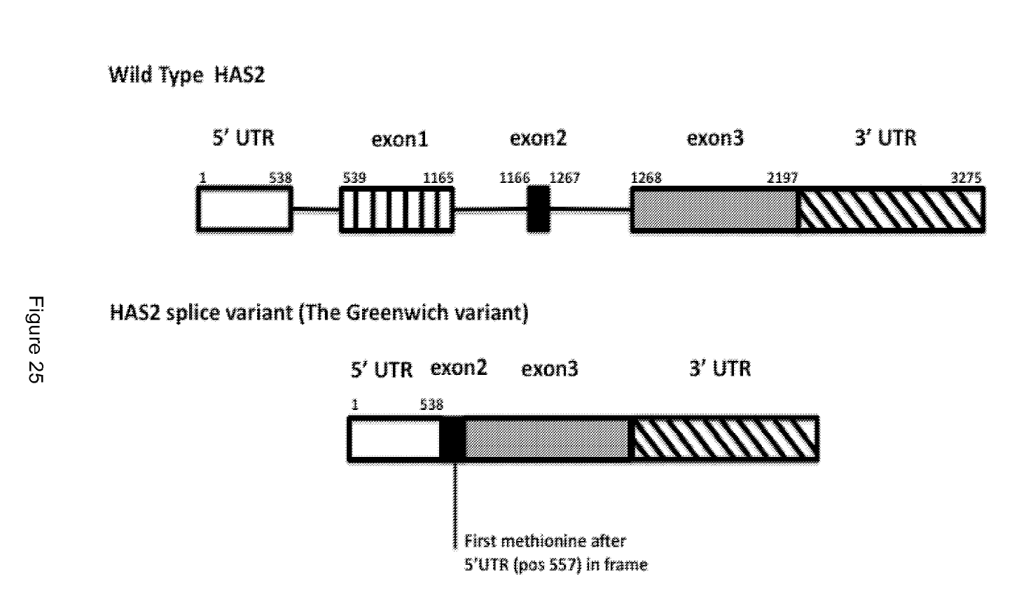

Catena-specific cell surface proteins were identified by gene array analysis using an Affymetrix GeneChip® Human Genome U133 Plus 2.0 Array as described in Example 22. The expression of selected CD proteins is shown in FIG. 24 for Ovcar3 catena (CSC 65%) and Ovcar3 epithelial monolayers (CSC 1%). Genes upregulated from 5-150 fold are in dark grey (red) and genes down regulated from 5-150 fold are in medium or light gray (green).

Receptors upregulated in CSC include CD220 (Insulin R), CD221 (IGF1R), CD222 (IGF2R), CD295 (Leptin R), CD331 (FGFR1), CD71 (Transferrin receptor), CD166 (Mannose receptor), CDC323 (JAM3), CALCRL (Calcitonin receptor-like) and PDGFRA. Other CD markers selective upregulated on catena were CD90 (Thy1) and CD49f (α6 integrin). This transcriptome analysis further showed that CD49f, CD90, CD99, CD166 (a cleaved form of which was in the catenae secretome), IGF1R (CD221), IGF2R (CD222) and CALCRL (Calcitonin receptor-like) were strongly upregulated in Catenae (>5-100 fold) compared to Ovcar3-GTL epithelial monolayers.

CD genes that were downregulated on catenae but highly expressed on Ovcar3-GTL monolayers included CD58, CD74, CD 109, CD 118, CD 146, CD 148, CD 167, CD 168, CD200, CD205 CD322/JAM2 and JAM3 (junctional adhesion molecules), CD326/Ep-CAM. CD133 was not differentially expressed between differentiated cancer cells and cancer stem cells.

### 3. Catena-Specific Gene Expression Corresponding to Human Surfaceome Genes

Using the list of predicted human surfaceome genes described in De Cunha et al., 2009, the expression of cell surface proteins was examined for catena cells relative to mesenchymal and epithelial ovarian cancer monolayers, low malignancy potential (LMP) ovarian patient samples and normal ovaries. This analysis identified 28 cell surface proteins with transmembrane domains differentially upregulated in catena compared to other cell types as well as cell surface proteins differentially downregulated in catena cells compared to other cell types (Table 11).

TABLE 10
Surfaceome FACS Analysis for Catena and Monolayers

| | Ovcar3 | Ovcar3 | Ovcar5 | Ovcar5 |
|---|---|---|---|---|
| Antigen | catena | monolayer | catena | monolayer |
| Blood gp A | 0.0 | 0.2 | 0.0 | 0.1 |
| Blood gp B(2) | 0.0 | 0.6 | 0.0 | 0.4 |
| CA125 (Muc16) | 0.0 | 98.5 | 0.0 | 0.1 |
| CAIX | 0.1 | 0.5 | 0.1 | 0.1 |
| CD133/1 | 1.0 | 0.4 | N/D | N/D |
| CD133/2 | 0.7 | 0.1 | N/D | N/D |
| CD150 | 0.2 | 0.2 | N/D | N/D |
| CD166 (ALCAM) | 98.0 | 0.1 | N/D | N/D |
| CD326 (EpCam) | 0.0 | 99.5 | 0.0 | 0.0 |
| CD34 | 0.7 | 0.0 | N/D | N/D |
| CD44 | 0.2 | 0.0 | N/D | N/D |
| CD49f (α6 integrin) | 95.0 | 0.0 | 98.1 | 99.0 |
| PDGR alpha | 99.0 | 1.0 | 95.0 | 16.0 |
| CD90 (Thy-1) | 97.6 | 0.0 | 99.9 | 99.5 |
| EGFRvIII | 0.1 | 0.3 | 0.0 | 0.1 |
| Endosialin | 0.1 | 0.4 | 0.0 | 0.1 |
| FAP-α | 0.0 | 0.3 | 0.0 | 0.1 |
| Folate Receptor α | 0.0 | 80.9 | 0.2 | 0.4 |
| FUCGM1 | 0.0 | 72.2 | 0.1 | 0.0 |
| GD2 | 0.2 | 1.3 | 0.1 | 0.2 |
| GD3 | 0.1 | 33.4 | 0.6 | 0.2 |
| GLOBO H | 0.3 | 1.9 | 1.8 | 1.3 |
| GM2 | 74.8 | 31.6 | 98.4 | 97.4 |
| GPA A33 antigen | 0.1 | 1.6 | 0.0 | 0.1 |
| KSA | 0.0 | 97.1 | 0.0 | 0.0 |
| LE Y | 0.1 | 95.7 | 0.0 | 0.0 |
| Lewisa | 0.0 | 28.7 | 0.0 | 0.1 |
| Lewisb | 0.0 | 70.2 | 0.0 | 0.1 |
| Lewisy (CD174) | 1.4 | 97.0 | 0.0 | 0.2 |
| MUC1 | 5.6 | 75.2 | 6.7 | 65.2 |
| PolySA | 0.0 | 63.6 | 0.0 | 0.0 |
| Sial-Lewisa | 0.0 | 15.7 | 0.0 | 0.1 |
| SLC34A2 | 0.5 | 98.7 | 0.1 | 0.2 |
| Sle a | 0.1 | 46.3 | 0.0 | 0.0 |
| sTn (CC49) | 0.2 | 63.9 | 1.3 | 1.1 |
| sTn (B72.3) | 0.1 | 32.7 | 0.1 | 1.2 |
| Sulfated Glycolipids | 0.2 | 1.3 | 0.1 | 0.4 |
| TF | 0.1 | 0.6 | 0.0 | 0.0 |
| TN | 0.1 | 8.8 | 0.1 | 0.1 |
| TRP-1 | 0.0 | 0.3 | 0.3 | 0.4 |
| Type 1 H | 0.0 | 0.4 | 0.0 | 0.1 |
| VEGFR1 | 0.0 | 0.1 | N/D | N/D |
| VEGFR2 | 0.0 | 0.1 | N/D | N/D |
| VEGFR3 | 0.0 | 0.1 | N/D | N/D |

TABLE 11
Differentially Expressed Catena-Specific Surface
Proteins in Predicted Human Surfacesome Gene Set

| | Upregulated Genes | Downregulated Genes |
|---|---|---|
| 1 | C10orf57 | ATP11C |
| 2 | CLCN5 | CACHD1 |
| 3 | CYP51A1 | CD44 |
| 4 | GPR98 | CD9 |
| 5 | GRAMD1C | CDH1 |
| 6 | HAS2 | CLDN3 |
| 7 | HHAT | CLDN4 |
| 8 | INSIG2 | CTAG1A |
| 9 | ITM2A | EPCAM |
| 10 | LPGAT1 | EPHA2 |
| 11 | MCOLN3 | FOLR1 |
| 12 | MFAP3L | MSLN |
| 13 | MXRA8 | MUC16 |
| 14 | NPFFR2 | PROM1 |
| 15 | PCDHB8 | SGMS2 |
| 16 | PDGFRA | SLC34A2 |
| 17 | PTDSS2 | TM9SF3 |
| 18 | SEMA6A | WT1 |
| 19 | SGCB | |
| 20 | SIDT1 | |
| 21 | SLC38A7 | |
| 22 | STRA6 | |
| 23 | TMEM35 | |
| 24 | TNFRSF19 | |
| 25 | TRPM6 | |
| 26 | XK | |
| 27 | ZDHHC13 | |
| 28 | ZPLD1 | |

### Example 28

### Culturing Catena in Serum-Free Media

The ability to culture catenae under defined conditions, (i.e., without serum) has several advantages, including allowing identification of autocrine pathways, identification of secreted proteins, and isolation and characterization of exosomes, all without contamination from serum components.

Catena cells were maintained in M5-FCS media. When cells reached a density of 200,000 cells/mL, the cells were pelleted at 300×g, washed twice with PBS to remove residual serum proteins and resuspended in serum-free M5 media with 1% P/S and recombinant insulin at 0.1 Uml (4.7 μg/mL final concentration) for growth. In the presence of insulin, catena cells maintain their morphology and proliferate at comparable rates to cells in serum-containing conditions.

### Example 29

### Analysis of Secreted and Exosomal Proteins from Catena

### 1. Isolation of Cell Fractions

To prepare secreted and exosomal fractions from catenae, the catenae were grown in serum-free media with insulin as described in Example 28 for 5 days, until the culture was near confluent. All centrifugations were done at 4° C. to preserve protein integrity. The cells were removed by centrifugation at 300×g for 10 min. The supernatant fraction was further spun down at 2,000×g for 20 minutes and at 10,000×g for 30 min to remove cellular debris. This supernatant was subjected to ultracentrifugation at100,000×g for 2.5 h. The new supernatant fraction, containing the soluble proteins secreted by catenae (i.e., the catena secretome) was concentrated 200-fold by through a 10 kDa molecular weight cutoff filter. The resulting pellet from the ultracentrifugation was washed twice with PBS, with each wash followed by another round of ultracentrifugation under the same conditions, and kept at 4° C. for further analysis. Exosomes were isolated from Ovcar5 and A2780 human ovarian cancer cell lines that were grown as attached mesenchymal monolayers and from two ovarian cancer patient ascites samples using the same methodology.

### 2. Characterization of Exosomes

Isolated exosomes were attached to poly-L-lysine coated slides, fixed with paraformaldehyde and glutaraldehyde, and visualized by SEM. The exosomes were round, 30-100 nm diameter structures. The hyaluronan-proteoglycan coat was visualized by SEM as described in Example 21. Exosomes were observed attached to the glycocalyx coat and could be released by hyaluronidase treatment.

We also analyzed the catena exosomes by transmission electron microscopy (TEM) to gain further insight into their structures.

For TEM, exosomes were attached to Formvar carbon-coated EM grids and stained with 2% Uranyl acetate solution for 15 minutes at room temperature. Under TEM, the catena exosomes had cup or saucer-shaped structures with hollow middles.

On a sucrose density gradient method, the catena exosomes were between 1.11-1.19 g/ml on the gradient.

### 3. FACS Analysis of Exosomes

Exosomes isolated as described above were adsorbed to 4 μM latex aldehyde/sulfate beads (Invitrogen) for 2 hours at room temperature. A wash was done with 1 M glycine to prevent non-specific binding of antibodies to unoccupied sites on the beads, and additional washes were followed by an incubation with fluorochrome-coupled antibodies. Using FACS analysis, catena exosomes were positive for CD63 but negative for CD45 and CD9.

Further that treatment of catena cells with 10 uM phorbol 12-myristate 13-acetate (PMA) induced exosomes release, whereas blebbistatin inhibited exosome release. The exosome isolation followed by FACS analysis allows rapid analysis of ovarian cancer stem cell exosomes both quantitatively and qualitatively. This protocol of measuring cancer stem cell specific exosomes can be used for early detection of cancer stem cells or monitor cancer stem cell content during therapy using ascites fluid or peripheral blood plasma samples.

### 4. Exosomal Protein Content

The protein content of catena exosomes was analyzed by mass spectrometry. Exosomes were resuspended in reducing sample buffer (Invitrogen) for standard gel electrophoresis and exosomal proteins were separated by electrophoresis in a 4-12% polyacrylamide gel electrophoresis. The proteins were visualized by Coomassie blue (Simply Blue-Invitrogen; a stain compatible with mass spectrometry). The protein lane containing exosomal proteins was cut into 15 pieces, the proteins extracted from each piece and protein content was analyzed by mass spectrometry.

Table 12 lists the proteins with more than 3 assigned peptide sequences at >95% confidence as identified by mass spectrometry of the catena exosomes. Table 13 lists the proteins present at higher (positive) amounts in catena exosomes relative to ovarian mesenchymal monolayer exosomes. The accession numbers listed in these tables and others in this example are from the International Protein Index (Kersey et al. 2004). The composition of the catena exosomes was similar to the human exosomes described by Simpson et al. 2008.

TABLE 12
Proteins Identified by Mass Spectrometry of Catena Exosomes
(begins on next page)

| | Accesssion | Identified |
|---|---|---|
| | Number | Spectra count |
| 1 | IPI00029715 | 101 |
| 2 | IPI00916356 | 98 |
| 3 | IPI00021439 | 62 |
| 4 | IPI00024067 | 56 |
| 5 | IPI00003865 | 55 |
| 6 | IPI00304962 | 51 |
| 7 | IPI00414676 | 45 |
| 8 | IPI00006482 | 42 |
| 9 | IPI00329801 | 37 |
| 10 | IPI00011654 | 35 |
| 11 | IPI00396485 | 35 |
| 12 | IPI00418471 | 34 |
| 13 | IPI00217994 | 33 |
| 14 | IPI00455315 | 33 |
| 15 | IPI00465248 | 32 |
| 16 | IPI00221226 | 31 |
| 17 | IPI00007960 | 28 |
| 18 | IPI00021842 | 27 |
| 19 | IPI00453473 | 27 |
| 20 | IPI00219018 | 25 |
| 21 | IPI00218343 | 23 |
| 22 | IPI00027493 | 22 |
| 23 | IPI00000816 | 21 |
| 24 | IPI00021263 | 21 |
| 25 | IPI00026272 | 21 |
| 26 | IPI00003362 | 20 |
| 27 | IPI00022048 | 20 |
| 28 | IPI00013933 | 20 |
| 29 | IPI00291175 | 19 |
| 30 | IPI00419585 | 19 |
| 31 | IPI00179330 | 19 |
| 32 | IPI00303476 | 19 |
| 33 | IPI00217563 | 19 |
| 34 | IPI00479186 | 18 |
| 35 | IPI00246058 | 18 |
| 36 | IPI00219217 | 17 |
| 37 | IPI00007765 | 17 |
| 38 | IPI00019502 | 16 |
| 39 | IPI00019906 | 16 |
| 40 | IPI00026781 | 15 |
| 41 | IPI00006707 | 15 |
| 42 | IPI00922108 | 15 |
| 43 | IPI00219365 | 14 |
| 44 | IPI00784154 | 14 |
| 45 | IPI00218487 | 14 |
| 46 | IPI00382470 | 13 |
| 47 | IPI00465439 | 13 |
| 48 | IPI00000874 | 12 |
| 49 | IPI00444262 | 12 |
| 50 | IPI00021428 | 12 |
| 51 | IPI00005996 | 12 |
| 52 | IPI00011253 | 11 |
| 53 | IPI00218918 | 11 |
| 54 | IPI00440493 | 11 |
| 55 | IPI00186290 | 10 |
| 56 | IPI00607708 | 10 |
| 57 | IPI00001639 | 9 |
| 58 | IPI00220301 | 9 |
| 59 | IPI00465028 | 9 |
| 60 | IPI00219757 | 9 |
| 61 | IPI00413108 | 9 |
| 62 | IPI00216088 | 9 |
| 63 | IPI00008530 | 9 |
| 64 | IPI00009342 | 9 |
| 65 | IPI00219839 | 9 |
| 66 | IPI00016342 | 9 |
| 67 | IPI00022462 | 9 |
| 68 | IPI00925214 | 9 |
| 69 | IPI00306046 | 9 |
| 70 | IPI00645078 | 8 |
| 71 | IPI00022774 | 8 |
| 72 | IPI00026216 | 8 |
| 73 | IPI00646304 | 8 |
| 74 | IPI00216691 | 8 |
| 75 | IPI00012011 | 8 |
| 76 | IPI00843975 | 8 |
| 77 | IPI00290770 | 8 |
| 78 | IPI00019472 | 8 |
| 79 | IPI00021695 | 8 |
| 80 | IPI00169383 | 7 |
| 81 | IPI00604590 | 7 |
| 82 | IPI00001734 | 7 |
| 83 | IPI00025252 | 7 |
| 84 | IPI00299738 | 7 |
| 85 | IPI00013894 | 7 |
| 86 | IPI00064795 | 7 |
| 87 | IPI00027626 | 7 |
| 88 | IPI00152906 | 7 |
| 89 | IPI00008524 | 7 |
| 90 | IPI00026087 | 7 |
| 91 | IPI00743335 | 7 |
| 92 | IPI00410034 | 7 |
| 93 | IPI00095891 | 7 |
| 94 | IPI00008557 | 7 |
| 95 | IPI00020984 | 7 |
| 96 | IPI00157144 | 7 |
| 97 | IPI00018352 | 6 |
| 98 | IPI00302925 | 6 |
| 99 | IPI00216318 | 6 |
| 100 | IPI00396378 | 6 |
| 101 | IPI00215914 | 6 |
| 102 | IPI00221091 | 6 |
| 103 | IPI00027547 | 6 |
| 104 | IPI00908739 | 6 |
| 105 | IPI00028714 | 6 |
| 106 | IPI00026268 | 6 |
| 107 | IPI00470535 | 6 |
| 108 | IPI00015148 | 6 |
| 109 | IPI00306604 | 6 |
| 110 | IPI00022649 | 6 |
| 111 | IPI00013890 | 6 |
| 112 | IPI00021033 | 5 |
| 113 | IPI00026314 | 5 |
| 114 | IPI00031461 | 5 |
| 115 | IPI00374563 | 5 |
| 116 | IPI00015102 | 5 |
| 117 | IPI00010896 | 5 |
| 118 | IPI00010720 | 5 |
| 119 | IPI00215780 | 5 |
| 120 | IPI00220642 | 5 |
| 121 | IPI00008964 | 5 |
| 122 | IPI00221222 | 5 |
| 123 | IPI00909337 | 5 |
| 124 | IPI00000856 | 5 |
| 125 | IPI00021266 | 5 |
| 126 | IPI00795676 | 5 |
| 127 | IPI00219221 | 5 |
| 128 | IPI00000190 | 5 |
| 129 | IPI00025277 | 5 |
| 130 | IPI00798360 | 5 |
| 131 | IPI00217519 | 5 |
| 132 | IPI00159899 | 5 |
| 133 | IPI00021405 | 5 |
| 134 | IPI00019997 | 5 |
| 135 | IPI00290928 | 5 |
| 136 | IPI00028055 | 5 |
| 137 | IPI00013808 | 4 |
| 138 | IPI00643920 | 4 |
| 139 | IPI00001508 | 4 |
| 140 | IPI00032292 | 4 |
| 141 | IPI00009802 | 4 |
| 142 | IPI00025491 | 4 |
| 143 | IPI00304925 | 4 |
| 144 | IPI00291006 | 4 |
| 145 | IPI00007752 | 4 |
| 146 | IPI00024933 | 4 |
| 147 | IPI00216587 | 4 |
| 148 | IPI00022434 | 4 |
| 149 | IPI00376798 | 4 |
| 150 | IPI00030179 | 4 |
| 151 | IPI00217030 | 4 |
| 152 | IPI00026271 | 4 |
| 153 | IPI00215918 | 4 |
| 154 | IPI00008527 | 4 |
| 155 | IPI00465361 | 4 |
| 156 | IPI00419880 | 4 |
| 157 | IPI00021536 | 4 |
| 158 | IPI00410714 | 4 |
| 159 | IPI00056478 | 4 |
| 160 | IPI00010697 | 4 |
| 161 | IPI00478231 | 4 |
| 162 | IPI00009607 | 4 |
| 163 | IPI00215998 | 4 |
| 164 | IPI00024650 | 4 |
| 165 | IPI00003373 | 4 |
| 166 | IPI00021048 | 4 |
| 167 | IPI00156689 | 4 |
| 168 | IPI00008986 | 4 |
| 169 | IPI00030362 | 4 |
| 170 | IPI00215790 | 4 |
| 171 | IPI00029737 | 4 |
| 172 | IPI00293665 | 4 |
| 173 | IPI00029739 | 3 |
| 174 | IPI00013508 | 3 |
| 175 | IPI00027350 | 3 |
| 176 | IPI00289499 | 3 |
| 177 | IPI00221092 | 3 |
| 178 | IPI00290566 | 3 |
| 179 | IPI00848226 | 3 |
| 180 | IPI00397801 | 3 |
| 181 | IPI00289819 | 3 |
| 182 | IPI00375236 | 3 |
| 183 | IPI00749183 | 3 |
| 184 | IPI00220362 | 3 |
| 185 | IPI00216319 | 3 |
| 186 | IPI00655650 | 3 |
| 187 | IPI00012493 | 3 |
| 188 | IPI00644127 | 3 |
| 189 | IPI00924764 | 3 |
| 190 | IPI00215965 | 3 |
| 191 | IPI00003269 | 3 |
| 192 | IPI00013895 | 3 |
| 193 | IPI00027462 | 3 |
| 194 | IPI00168325 | 3 |
| 195 | IPI00103481 | 3 |
| 196 | IPI00384662 | 3 |
| 197 | IPI00306332 | 3 |
| 198 | IPI00052885 | 3 |
| 199 | IPI00178100 | 3 |
| 200 | IPI00015614 | 3 |
| 201 | IPI00007047 | 3 |
| 202 | IPI00456899 | 3 |
| 203 | IPI00644297 | 3 |
| 204 | IPI00916126 | 3 |
| 205 | IPI00900311 | 3 |
| 206 | IPI00220194 | 3 |
| 207 | IPI00010271 | 3 |
| 208 | IPI00215920 | 3 |
| 209 | IPI00793199 | 3 |
| 210 | IPI00410666 | 3 |
| 211 | IPI00480022 | 3 |
| 212 | IPI00002460 | 3 |
| 213 | IPI00025512 | 3 |
| 214 | IPI00000005 | 3 |
| 215 | IPI00300096 | 3 |
| 216 | IPI00008167 | 3 |
| 217 | IPI00549343 | 3 |
| 218 | IPI00021983 | 3 |
| 219 | IPI00013744 | 3 |
| 220 | IPI00554711 | 3 |
| 221 | IPI00003348 | 3 |
| 222 | IPI00071509 | 3 |
| 223 | IPI00046633 | 3 |
| 224 | IPI00644467 | 3 |

TABLE 13
Proteins Present in Greater Amounts in catena Exosomes
Relative to Ovarian Mesenchymal Monolayer Exosomes
Proteins

| | |
|---|---|
| CD63 | FREM2 |
| CLDN6 | IGF2R |
| ANXA1 | SEMA6A |
| COL1A2 | SLC3A2 |
| TMED10 | TFRC |
| NEO1 | PKM2 |
| PRTG | KPNB1 |
| ALCAM | XPO1 |
| DAG1 | |

### 5. Secretome Protein Content

Using mass spectrometry analysis we have identified 210 proteins secreted by catena cells in serum-free protein-media with insulin. Table 14 lists the proteins with more than 10 assigned peptide sequences (>95% confidence) identified by mass spectrometry of catena secretome. The secretome of two other ovarian mesenchymal cancer cell lines (A2780 and Ovcar5) were also analyzed by mass spectrometry. Table 15 lists the proteins that were produced at higher amounts by catena cells relative to differentiated mesenchymal monolayer cells as determined by gene expression or mass spectrometry analysis.

Mass spectrometry analysis of catena secretome also showed that catenae produced up to 500-fold more COL1A2 (Collagen Type1 apha2) than mesenchymal ovarian cancer monolayers.

TABLE 14
Proteins Identified by Mass Spectrometry of Catena Secretome

| | Accession | Identified |
|---|---|---|
| | Number | Spectra count |
| 1 | IPI00304962 | 1514 |
| 2 | IPI00414676 | 432 |
| 3 | IPI00003865 | 186 |
| 4 | IPI00180707 | 183 |
| 5 | IPI00007960 | 178 |
| 6 | IPI00916356 | 173 |
| 7 | IPI00021439 | 152 |
| 8 | IPI00021033 | 149 |
| 9 | IPI00465248 | 149 |
| 10 | IPI00291175 | 123 |
| 11 | IPI00024067 | 114 |
| 12 | IPI00026781 | 104 |
| 13 | IPI00418471 | 103 |
| 14 | IPI00021842 | 90 |
| 15 | IPI00011654 | 84 |
| 16 | IPI00013808 | 84 |
| 17 | IPI00029739 | 83 |
| 18 | IPI00186290 | 82 |
| 19 | IPI00643920 | 80 |
| 20 | IPI00739099 | 79 |
| 21 | IPI00419585 | 73 |
| 22 | IPI00218343 | 72 |
| 23 | IPI00645078 | 72 |
| 24 | IPI00006114 | 70 |
| 25 | IPI00002966 | 69 |
| 26 | IPI00022774 | 64 |
| 27 | IPI00643034 | 62 |
| 28 | IPI00026944 | 62 |
| 29 | IPI00003362 | 60 |
| 30 | IPI00001639 | 58 |
| 31 | IPI00219217 | 57 |
| 32 | IPI00479186 | 57 |
| 33 | IPI00001508 | 57 |
| 34 | IPI00382470 | 56 |
| 35 | IPI00219018 | 55 |
| 36 | IPI00169383 | 53 |
| 37 | IPI00021290 | 52 |
| 38 | IPI00000874 | 51 |
| 39 | IPI00607708 | 51 |
| 40 | IPI00220301 | 51 |
| 41 | IPI00026216 | 49 |
| 42 | IPI00646304 | 48 |
| 43 | IPI00032292 | 48 |
| 44 | IPI00029715 | 47 |
| 45 | IPI00444262 | 45 |
| 46 | IPI00018352 | 45 |
| 47 | IPI00000816 | 44 |
| 48 | IPI00465439 | 43 |
| 49 | IPI00216691 | 42 |
| 50 | IPI00465028 | 42 |
| 51 | IPI00219365 | 42 |
| 52 | IPI00026314 | 42 |
| 53 | IPI00604590 | 41 |
| 54 | IPI00001734 | 39 |
| 55 | IPI00009802 | 36 |
| 56 | IPI00396485 | 36 |
| 57 | IPI00178352 | 36 |
| 58 | IPI00027192 | 36 |
| 59 | IPI00013508 | 36 |
| 60 | IPI00010796 | 35 |
| 61 | IPI00179953 | 35 |
| 62 | IPI00219757 | 34 |
| 63 | IPI00302592 | 33 |
| 64 | IPI00291866 | 33 |
| 65 | IPI00022744 | 33 |
| 66 | IPI00025252 | 32 |
| 67 | IPI00179330 | 31 |
| 68 | IPI00003590 | 31 |
| 69 | IPI00479306 | 31 |
| 70 | IPI00004534 | 31 |
| 71 | IPI00298281 | 30 |
| 72 | IPI00031030 | 30 |
| 73 | IPI00218319 | 30 |
| 74 | IPI00027230 | 30 |
| 75 | IPI00012011 | 29 |
| 76 | IPI00453476 | 29 |
| 77 | IPI00219029 | 29 |
| 78 | IPI00010800 | 29 |
| 79 | IPI00009904 | 29 |
| 80 | IPI00027497 | 27 |
| 81 | IPI00783313 | 27 |
| 82 | IPI00028908 | 26 |
| 83 | IPI00220834 | 26 |
| 84 | IPI00003919 | 25 |
| 85 | IPI00031461 | 25 |
| 86 | IPI00217994 | 25 |
| 87 | IPI00784154 | 25 |
| 88 | IPI00014572 | 25 |
| 89 | IPI00555956 | 25 |
| 90 | IPI00301263 | 25 |
| 91 | IPI00027442 | 25 |
| 92 | IPI00298961 | 25 |
| 93 | IPI00329633 | 25 |
| 94 | IPI00413959 | 24 |
| 95 | IPI00291136 | 24 |
| 96 | IPI00296922 | 23 |
| 97 | IPI00298994 | 23 |
| 98 | IPI00219525 | 23 |
| 99 | IPI00100160 | 23 |
| 100 | IPI00021263 | 22 |
| 101 | IPI00027350 | 22 |
| 102 | IPI00019502 | 22 |
| 103 | IPI00025019 | 22 |
| 104 | IPI00025491 | 22 |
| 105 | IPI00291922 | 22 |
| 106 | IPI00289499 | 22 |
| 107 | IPI00016636 | 22 |
| 108 | IPI00374563 | 21 |
| 109 | IPI00009943 | 21 |
| 110 | IPI00302925 | 21 |
| 111 | IPI00383581 | 21 |
| 112 | IPI00012268 | 21 |
| 113 | IPI00015102 | 21 |
| 114 | IPI00024664 | 21 |
| 115 | IPI00006608 | 20 |
| 116 | IPI00456969 | 20 |
| 117 | IPI00641829 | 20 |
| 118 | IPI00218493 | 20 |
| 119 | IPI00299738 | 19 |
| 120 | IPI00291005 | 19 |
| 121 | IPI00013894 | 19 |
| 122 | IPI00413108 | 19 |
| 123 | IPI00023673 | 19 |
| 124 | IPI00012007 | 19 |
| 125 | IPI00298547 | 18 |
| 126 | IPI00937615 | 18 |
| 127 | IPI00293464 | 18 |
| 128 | IPI00018931 | 18 |
| 129 | IPI00017672 | 17 |
| 130 | IPI00020599 | 17 |
| 131 | IPI00024175 | 17 |
| 132 | IPI00793443 | 17 |
| 133 | IPI00783097 | 17 |
| 134 | IPI00291419 | 17 |
| 135 | IPI00216318 | 16 |
| 136 | IPI00304925 | 16 |
| 137 | IPI00028004 | 16 |
| 138 | IPI00218993 | 16 |
| 139 | IPI00219910 | 16 |
| 140 | IPI00016832 | 16 |
| 141 | IPI00744692 | 16 |
| 142 | IPI00218342 | 16 |
| 143 | IPI00220740 | 16 |
| 144 | IPI00843975 | 15 |
| 145 | IPI00011253 | 15 |
| 146 | IPI00220766 | 15 |
| 147 | IPI00029623 | 15 |
| 148 | IPI00296537 | 14 |
| 149 | IPI00021428 | 14 |
| 150 | IPI00028911 | 14 |
| 151 | IPI00396378 | 14 |
| 152 | IPI00219446 | 14 |
| 153 | IPI00021700 | 14 |
| 154 | IPI00007423 | 14 |
| 155 | IPI00023601 | 14 |
| 156 | IPI00395783 | 14 |
| 157 | IPI00168884 | 14 |
| 158 | IPI00219622 | 14 |
| 159 | IPI00064795 | 14 |
| 160 | IPI00644712 | 14 |
| 161 | IPI00418262 | 14 |
| 162 | IPI00007402 | 14 |
| 163 | IPI00020672 | 14 |
| 164 | IPI00305692 | 14 |
| 165 | IPI00003949 | 13 |
| 166 | IPI00291006 | 13 |
| 167 | IPI00216088 | 13 |
| 168 | IPI00007752 | 13 |
| 169 | IPI00010896 | 13 |
| 170 | IPI00019600 | 13 |
| 171 | IPI00009032 | 13 |
| 172 | IPI00296913 | 13 |
| 173 | IPI00294004 | 13 |
| 174 | IPI00027223 | 13 |
| 175 | IPI00397526 | 13 |
| 176 | IPI00005614 | 12 |
| 177 | IPI00643041 | 12 |
| 178 | IPI00008530 | 12 |
| 179 | IPI00291262 | 12 |
| 180 | IPI00010720 | 12 |
| 181 | IPI00297779 | 12 |
| 182 | IPI00221092 | 12 |
| 183 | IPI00002211 | 12 |
| 184 | IPI00177728 | 12 |
| 185 | IPI00185146 | 12 |
| 186 | IPI00010706 | 12 |
| 187 | IPI00843765 | 11 |
| 188 | IPI00216049 | 11 |
| 189 | IPI00012069 | 11 |
| 190 | IPI00299000 | 11 |
| 191 | IPI00290566 | 11 |
| 192 | IPI00215780 | 11 |
| 193 | IPI00299608 | 11 |
| 194 | IPI00300371 | 11 |
| 195 | IPI00022200 | 10 |
| 196 | IPI00032293 | 10 |
| 197 | IPI00003815 | 10 |
| 198 | IPI00290770 | 10 |
| 199 | IPI00376005 | 10 |
| 200 | IPI00006707 | 10 |
| 201 | IPI00024933 | 10 |
| 202 | IPI00299571 | 10 |
| 203 | IPI00024911 | 10 |
| 204 | IPI00171199 | 10 |
| 205 | IPI00021370 | 10 |
| 206 | IPI00438229 | 10 |
| 207 | IPI00006052 | 10 |
| 208 | IPI00479786 | 10 |
| 209 | IPI00184330 | 10 |

TABLE 15
Proteins Present in Greater Amounts in the Catena Secretome
Relative to the Ovarian Mesenchymal Monolayer Secretome
Protein

| | | |
|---|---|---|
| ACLY | FLNA | NPEPPS |
| ACTN1 | FLNC | NTS |
| ACTN4 | FOLH1 | PDIA3 |
| ANGPT1 | FREM2 | PGK1 |
| APOE | GDI2 | PKM2 |
| ATIC | GOT1 | PLOD1 |
| CAD | GSN | PLTP |
| CARBP1 | HAPLN1 | POSTN |
| CFH | HSP90AA1 | PRTG |
| CLDN23 | HSP90AB1 | SPAG9 |
| CLDN6 | HSP90B1 | TEX 15 |
| CLDN7 | HSPA4 | TKT |
| CLTC | HSPA5 | TLN1 |
| COL1A2 | HSPA8 | TRUB1 |
| COL3A1 | INHBE | TUBA1C |
| COL5A2 | KPNB1 | TUBB |
| CSE1L | MSN | UBA1 |
| DDB1 | NASP | UBTD2 |
| EEF2 | NCL | VCL |
| ENO1 | NEO1 | VCP |
| EZR | NES | VIM |
| FASN | NID1 | XPO1 |
| | | YWHAE |

### 6. Catena Cell Surface Proteins

Membrane proteins were isolated from catena cells by phase partitioning using the nonionic detergent Triton X-114. Catena cells were cultured in serum-free protein-media with insulin for 5 days as described in Example 28. Cells were pelleted by centrifugation at 1500 rpm for 10 minutes at room temperature. The Triton X-114 soluble membrane proteins (catena surfaceome) were separated from the cell lysate by phase partitioning technique (Bordier 1981) and subjected to mass spectrometry. Table 16 lists the proteins with more than 3 assigned peptide sequences (>95% confidence) in the catena cells.

TABLE 16
Proteins Identified by Mass Spectrometry
of Catena Membrane Proteins

| | Accession | Identified |
|---|---|---|
| | Number | Spectra Count |
| 1 | IPI00418471 | 314 |
| 2 | IPI00021440 | 175 |
| 3 | IPI00220327 | 162 |
| 4 | IPI00021304 | 161 |
| 5 | IPI00218343 | 156 |
| 6 | IPI00009865 | 117 |
| 7 | IPI00011654 | 101 |
| 8 | IPI00296337 | 94 |
| 9 | IPI00001159 | 83 |
| 10 | IPI00303476 | 75 |
| 11 | IPI00440493 | 71 |
| 12 | IPI00010800 | 53 |
| 13 | IPI00398002 | 47 |
| 14 | IPI00216308 | 41 |
| 15 | IPI00022744 | 36 |
| 16 | IPI00025874 | 35 |
| 17 | IPI00298961 | 32 |
| 18 | IPI00216230 | 29 |
| 19 | IPI00028635 | 28 |
| 20 | IPI00003865 | 27 |
| 21 | IPI00019359 | 26 |
| 22 | IPI00007402 | 26 |
| 23 | IPI00009960 | 23 |
| 24 | IPI00006482 | 23 |
| 25 | IPI00177817 | 22 |
| 26 | IPI00007188 | 21 |
| 27 | IPI00024067 | 21 |
| 28 | IPI00793443 | 19 |
| 29 | IPI00185146 | 18 |
| 30 | IPI00027493 | 18 |
| 31 | IPI00304596 | 18 |
| 32 | IPI00027252 | 17 |
| 33 | IPI00414676 | 17 |
| 34 | IPI00008964 | 16 |
| 35 | IPI00157790 | 14 |
| 36 | IPI00170692 | 14 |
| 37 | IPI00640703 | 14 |
| 38 | IPI00010740 | 14 |
| 39 | IPI00022462 | 14 |
| 40 | IPI00783271 | 14 |
| 41 | IPI00024145 | 13 |
| 42 | IPI00465248 | 13 |
| 43 | IPI00020984 | 13 |
| 44 | IPI00306382 | 13 |
| 45 | IPI00001639 | 13 |
| 46 | IPI00022202 | 12 |
| 47 | IPI00003362 | 12 |
| 48 | IPI00009368 | 12 |
| 49 | IPI00007765 | 11 |
| 50 | IPI00024364 | 11 |
| 51 | IPI00291175 | 11 |
| 52 | IPI00479786 | 10 |
| 53 | IPI00444262 | 10 |
| 54 | IPI00334190 | 10 |
| 55 | IPI00216484 | 10 |
| 56 | IPI00019472 | 9 |
| 57 | IPI00009867 | 9 |
| 58 | IPI00297084 | 9 |
| 59 | IPI00396485 | 9 |
| 60 | IPI00219018 | 9 |
| 61 | IPI00017334 | 8 |
| 62 | IPI00010349 | 8 |
| 63 | IPI00024650 | 8 |
| 64 | IPI00215965 | 8 |
| 65 | IPI00604664 | 8 |
| 66 | IPI00007084 | 8 |
| 67 | IPI00008557 | 8 |
| 68 | IPI00031397 | 8 |
| 69 | IPI00003833 | 8 |
| 70 | IPI00219729 | 8 |
| 71 | IPI00008529 | 8 |
| 72 | IPI00024976 | 8 |
| 73 | IPI00015972 | 7 |
| 74 | IPI00028357 | 7 |
| 75 | IPI00031410 | 7 |
| 76 | IPI00784154 | 7 |
| 77 | IPI00005737 | 7 |
| 78 | IPI00020436 | 7 |
| 79 | IPI00021263 | 7 |
| 80 | IPI00395887 | 7 |
| 81 | IPI00419585 | 7 |
| 82 | IPI00216691 | 7 |
| 83 | IPI00008998 | 7 |
| 84 | IPI00016342 | 7 |
| 85 | IPI00302592 | 7 |
| 86 | IPI00305383 | 7 |
| 87 | IPI00026824 | 7 |
| 88 | IPI00328415 | 7 |
| 89 | IPI00291467 | 7 |
| 90 | IPI00006579 | 6 |
| 91 | IPI00011253 | 6 |
| 92 | IPI00383581 | 6 |
| 93 | IPI00219291 | 6 |
| 94 | IPI00032003 | 6 |
| 95 | IPI00179330 | 6 |
| 96 | IPI00028055 | 6 |
| 97 | IPI00026781 | 6 |
| 98 | IPI00218319 | 6 |
| 99 | IPI00295772 | 6 |
| 100 | IPI00329801 | 6 |
| 101 | IPI00465128 | 6 |
| 102 | IPI00009950 | 6 |
| 103 | IPI00015102 | 6 |
| 104 | IPI00021954 | 6 |
| 105 | IPI00025491 | 6 |
| 106 | IPI00027448 | 6 |
| 107 | IPI00027505 | 6 |
| 108 | IPI00418497 | 6 |
| 109 | IPI00000816 | 6 |
| 110 | IPI00012048 | 6 |
| 111 | IPI00470467 | 6 |
| 112 | IPI00410034 | 6 |
| 113 | IPI00242630 | 5 |
| 114 | IPI00156374 | 5 |
| 115 | IPI00220835 | 5 |
| 116 | IPI00003968 | 5 |
| 117 | IPI00014053 | 5 |
| 118 | IPI00029133 | 5 |
| 119 | IPI00015833 | 5 |
| 120 | IPI00002214 | 5 |
| 121 | IPI00011635 | 5 |
| 122 | IPI00019502 | 5 |
| 123 | IPI00022143 | 5 |
| 124 | IPI00219217 | 5 |
| 125 | IPI00220416 | 5 |
| 126 | IPI00306518 | 5 |
| 127 | IPI00007752 | 5 |
| 128 | IPI00008524 | 5 |
| 129 | IPI00032325 | 5 |
| 130 | IPI00169383 | 5 |
| 131 | IPI00302458 | 5 |
| 132 | IPI00554648 | 5 |
| 133 | IPI00015148 | 5 |
| 134 | IPI00016339 | 5 |
| 135 | IPI00411639 | 5 |
| 136 | IPI00008986 | 5 |
| 137 | IPI00022434 | 5 |
| 138 | IPI00375441 | 4 |
| 139 | IPI00290770 | 4 |
| 140 | IPI00465028 | 4 |
| 141 | IPI00014230 | 4 |
| 142 | IPI00297492 | 4 |
| 143 | IPI00011200 | 4 |
| 144 | IPI00025796 | 4 |
| 145 | IPI00031804 | 4 |
| 146 | IPI00306290 | 4 |
| 147 | IPI00453473 | 4 |
| 148 | IPI00306505 | 4 |
| 149 | IPI00029628 | 4 |
| 150 | IPI00017895 | 4 |
| 151 | IPI00219685 | 4 |
| 152 | IPI00019906 | 4 |
| 153 | IPI00220739 | 4 |
| 154 | IPI00013917 | 4 |
| 155 | IPI00002412 | 4 |
| 156 | IPI00021048 | 4 |
| 157 | IPI00027438 | 4 |
| 158 | IPI00029019 | 4 |
| 159 | IPI00031522 | 4 |
| 160 | IPI00171903 | 4 |
| 161 | IPI00006211 | 4 |
| 162 | IPI00012069 | 4 |
| 163 | IPI00016670 | 4 |
| 164 | IPI00023860 | 4 |
| 165 | IPI00026087 | 4 |
| 166 | IPI00186290 | 4 |
| 167 | IPI00215918 | 4 |
| 168 | IPI00412713 | 4 |
| 169 | IPI00020944 | 4 |
| 170 | IPI00023526 | 4 |
| 171 | IPI00100656 | 4 |
| 172 | IPI00220740 | 4 |
| 173 | IPI00292135 | 4 |
| 174 | IPI00419258 | 3 |
| 175 | IPI00410714 | 3 |
| 176 | IPI00374208 | 3 |
| 177 | IPI00395769 | 3 |
| 178 | IPI00013678 | 3 |
| 179 | IPI00384444 | 3 |
| 180 | IPI00025252 | 3 |
| 181 | IPI00644712 | 3 |
| 182 | IPI00001636 | 3 |
| 183 | IPI00012490 | 3 |
| 184 | IPI00220644 | 3 |
| 185 | IPI00018352 | 3 |
| 186 | IPI00027107 | 3 |
| 187 | IPI00549343 | 3 |
| 188 | IPI00002188 | 3 |
| 189 | IPI00003881 | 3 |
| 190 | IPI00003964 | 3 |
| 191 | IPI00007611 | 3 |
| 192 | IPI00008530 | 3 |
| 193 | IPI00014235 | 3 |
| 194 | IPI00025086 | 3 |
| 195 | IPI00026154 | 3 |
| 196 | IPI00026268 | 3 |
| 197 | IPI00026942 | 3 |
| 198 | IPI00027497 | 3 |
| 199 | IPI00027547 | 3 |
| 200 | IPI00216049 | 3 |
| 201 | IPI00219219 | 3 |
| 202 | IPI00294911 | 3 |
| 203 | IPI00297261 | 3 |
| 204 | IPI00374975 | 3 |
| 205 | IPI00382470 | 3 |
| 206 | IPI00418169 | 3 |
| 207 | IPI00465439 | 3 |
| 208 | IPI00550165 | 3 |
| 209 | IPI00844578 | 3 |
| 210 | IPI00884105 | 3 |
| 211 | IPI00027078 | 3 |
| 212 | IPI00554590 | 3 |
| 213 | IPI00100160 | 3 |
| 214 | IPI00005719 | 3 |
| 215 | IPI00005728 | 3 |
| 216 | IPI00006865 | 3 |
| 217 | IPI00012066 | 3 |
| 218 | IPI00014149 | 3 |
| 219 | IPI00019353 | 3 |
| 220 | IPI00021805 | 3 |
| 221 | IPI00023001 | 3 |
| 222 | IPI00023542 | 3 |
| 223 | IPI00026272 | 3 |
| 224 | IPI00028031 | 3 |
| 225 | IPI00029264 | 3 |
| 226 | IPI00030363 | 3 |
| 227 | IPI00032150 | 3 |
| 228 | IPI00045921 | 3 |
| 229 | IPI00063903 | 3 |
| 230 | IPI00156689 | 3 |
| 231 | IPI00220642 | 3 |
| 232 | IPI00293946 | 3 |
| 233 | IPI00299084 | 3 |
| 234 | IPI00303954 | 3 |
| 235 | IPI00333619 | 3 |
| 236 | IPI00848226 | 3 |

### Example 30

### Identification of a HAS2 Splice Variant

Catena mRNA was prepared as described in Example 22, converted to cDNA and subjected to 454 deep sequencing and analysis on the Genome Sequencer FLX system and software according to the manufacturer's instructions. The alignment of sequence reads from the catena mRNA against the wild-type (wt) HAS2 sequence showed a heterogeneous distribution with more coverage from the 5′ UTR and exon3. These results suggested the presence of a HAS2 splice variant expressed in catenae.

To identify the splice variant, a set of forward and reverse PCR primers were prepared from for the HAS2 mRNA 5′ UTR and 3′ UTR regions, respectively based on the human HAS2 gene sequence (NCBI Accession No. NM_{—}005328). The forward primer was located at position 487-509 and had the sequence CGGGACCACACAGACAGGCTGAG (SEQ ID NO. 1). The reverse primer was located at position 2202-2227 and had the sequence GTGTGACTGCAAACGTCAAAACATGG (SEQ ID NO. 2). The expected PCR amplification product for the wt HAS2 mRNA is 1741 bp. Using RT-PCR with catena mRNA, the amplification products produced the expected 1741 by fragment as well as an additional fragment at approximately 1100 bp. The smaller fragment was identified as an 1115 by fragment lacking exon1 of the HAS2 gene. This HAS2 splice variant has been designated as the Greenwich variant. The Greenwich variant contains an in-frame deletion and encodes a protein beginning at amino acid 215 of the wt HAS2 gene and ending amino acid 552 at the normal C terminus as shown in FIG. 25. Translation for this protein begins at nucleotide position 557 in exon2, which is the first methionine after the splice point.

HAS2 is a membrane-bound protein with a predicted structure of multiple membrane, cytoplasmic and extracellular domains as shown in the UniProtKB/Swiss-Prot database, ID No. Q92819 (http://www.uniprot.org/uniprot/Q92819). The HAS2 splice variant begins in the middle of the first cytoplasmic domain and retains several predicted membrane spanning domains.

### Example 31

### HAS2 and PDGFRA Expression in Ovarian Cancer Cell Lines and in Primary Tumors

mRNA prepared from Ovcar3 monolayers, Ovcar 5 monolayers and A2780 monolayers was analyzed for the presence of the HAS2 transcripts by RT-PCR using the PCR primer set of Example 30. Neither the wild type nor the splice variant transcript was detected in any of these cell lines.

Samples were obtained from peritoneal solid tumors from patients with advanced stage ovarian cancer. Of 220 tested samples, five had heterozygous missense mutations in the HAS2 gene. Four of the five mutations were located in exon1, near the exon1-exon2 junction (at position 954, 981, 1099 and 1136; the junction occurs at nucleotide 1165)). Such mutations could lead to the observed alternative splicing in catena HAS2 mRNA. The fifth mutation was located at position 2009 in exon3. HAS2 is located on chromosome 8 and nucleotides located at the mutations and normal alleles of the positive strand are listed below in Table 17. Mutational analysis of mRNA extracted from Ovcar3 catena cells is shown in Table 18. Analysis of total cellular RNA showed approximately equal representation of both alleles, whereas analysis of actively translated mRNA showed preferential translation of mutant mRNAs (96% mutant to 4% wt).

In Tables 17 and 18, chromosomal site refers to the nucleotide position on positive (+) strand of chromosome 8; the corresponding mRNA site or locations is also provided.

TABLE 17
HAS2 Mutations in Patient Samples

| Chromosomal | mRNA | Wild type | Tumor |
|---|---|---|---|
| Site | Site | Allele | Allele |
| 122710164 | 1136 | C | T |
| 122710201 | 1099 | C | A |
| 122710319 | 981 | C | T |
| 122710346 | 954 | C | A |
| 122695718 | 2009 | C | A |

TABLE 18
HAS2 Mutations in Isolated Catenae

| Chromosomal | mRNA | Reference | Tumor | Tumor |
|---|---|---|---|---|
| Site | position | Allele | Allele 1 | Allele 2 |
| 122722660 | 5′ UTR | A | G | G |
| 122722537 | 5′ UTR | A | T | T |
| 122696460 | Intron | C | C | G |
| 122696461 | Intron | T | T | A |

The SOLiD RNA Sequencing System (Applied Biosystems) was used to obtain the mutational profile of PDGFRA mRNA in catena cells and identified 5 homologous mutations (Table 19). These mutations were in 100% of the total and polysomal PDGFRA mRNA. In Table 19, the chromosomal site refers to the nucleotide position on +strand of chromosome 4; the corresponding mRNA location is also provided.

TABLE 19
PDGFRA Mutations in Isolated Catenae

| Chromosomal | mRNA | Reference | Tumor | Tumor |
|---|---|---|---|---|
| Site | position | Allele | Allele 1 | Allele 2 |
| 54858583 | Exon23 | T | G | G |
| 54857707 | Exon23 | C | A | A |
| 54834644 | Exon10 | G | A | A |
| 54828356 | Exon6 | A | T | T |
| 54828357 | Exon6 | A | T | T |

### REFERENCES

Amazonia!: An Online Resource to Google and Visualize Public Human whole Genome Expression Data. Tanguy Le Carrour, Said Assou, Sylvie Tondeur, Ludovic Lhermitte, Ned Lamb, Thierry Reme, Veronique Pantesco, Samir Hamamah, Bernard Klein, John De Vos.

The Open Bioinformatics Journal, 2010,4 : 5-10

Bapat S A, Mali A M, Koppikar C B, Kurrey N K. Stem and progenitorlike cells contribute to the aggressive behavior of human epithelial ovarian cancer. Cancer Res. 2005 Apr. 15; 65(8):3025-9.

Bardiès M., Thedrez P., Gestin J.-F., Marcille B.-M., Guerreau D., Faivre-Chauvet A., Use of multi-cell spheroids of ovarian carcinoma as an intraperitoneal radio-immunotherapy model: uptake, retention kinetics and dosimetric evaluation. Int. J. Cancer 50 1992:, pp. 984-991.

Becker L., Prewett T. L., Spaulding G. F. and Goodwin T. J. Threedimensional growth and differentiation of ovarian tumor cell line in high aspect rotating-wall vessel: morphologic and embryological considerations. J. Cell. Biochem. 51 (1993), pp. 283-289.

Bjorge L., Junnikkala S., Kristoffersen E. K., Hakulinen J., Matre R., Meri S. Resistance of ovarian teratocarcinoma cell spheroids to complement-mediated lysis. Br. J. Cancer, 1997: 75: 1247-1255

Bordier, C. 1981. Phase separation of integral membrane proteins in Triton X-114 solution. J. Biol. Chem. 256, 1604-1607.

Chignola R., Foroni R., Franceschi A., Pasti M., Candiani C., Anselmi C., Fracasso G., Tridente G., Colombatti M. Heterogeneous response of individual multicellular tumour spheroids to immunotoxins and ricin toxin. Br. J. Cancer, 1995: 72: 607-6

Davidson B. Biological characteristics of cancers involving the serosal cavities. Crit Rev Oncog. 2007; 13:189-227.

De Cunha et al., Bioinformatics construction of the human cell surfaceome. Proc Natl Acad Sci USA. 2009 Sep. 29; 106(39):16752-7.

Di Maria G U, Comba P, Malignant Pleural Mesothelioma: The Puzzling Role of Gene-Environment Interaction. Chest. 2004; 125:1604-1607.

Filipovich I. V., Sorokina N. I., Robillard N. and Chatal J.-F., Radiation-induced apoptosis in human ovarian carcinoma cells growing as a monolayer and as multicell spheroids. Int. J. Cancer 72 1997; pp. 851-859.

Friedl P Wolf K. Tumour-cell invasion and migration: diversity and escape mechanisms Nature Reviews Cancer 2003; 3:362-374.

Gorlach A., Herter P., Hentschel H., Frosch P. J., Acker H. Effects of nIFN β and rIFN on growth and morphology of two human melanoma cell lines: comparison between two- and three-dimensional culture. Int. J. Cancer, 56: 249-254, 1994.

Gregory P A, Bert A G, Paterson E L, Barry S C, Tsykin A, Farshid G, Vadas M A, Khew-Goodall Y, Goodall G J. The miR-200 family and miR-205 regulate epithelial to mesenchymal transition by targeting ZEB1 and SIP1. Nat Cell Biol. 2008 May;10(5):593-601.

Hamilton T C, Young R C, McKoy W M, Grotzinger K R, Green J A, Chu E W, Whang-Peng J, Rogan A M, Green W R, Ozols R F. Characterization of a human ovarian carcinoma cell line (NIH:OVCAR-3) with androgen and estrogen receptors. Cancer Res. 1983 November; 43(11):5379-89.

Jemal A, Siegel R, Ward E, Hao Y, Xu J, Murray T, Thun M J. Cancer statistics, 2008. CA Cancer J Clin. 2008 March-April; 58(2):71-96.

Kersey P. J., Duarte J., Williams A., Karavidopoulou Y., Birney E., Apweiler R. The International Protein Index: An integrated database for proteomics experiments. Proteomics 4(7): 1985-1988 (2004).

Ludwig, T. E., Bergendahl, V., Levenstein, M. E., Yu, J., Probasco, M. D. & Thompson. J. A. 2006. Feeder-independent Culruer of Human Embryonic Stem Cells. Nature Med 3: 637-646.

Makhija S., Taylor D. D., Gibb R. K. and Gerçel-Taylor C., Taxol-induced Bcl-2 phosphorylation in ovarian cancer cell monolayer and spheroids. Int. J. Oncol. 14 (1999), pp. 515-521.

Markman M, Markman J, Webster K, Zanotti K, Kulp B, Peterson G, Belinson J. Duration of response to second-line, platinum-based chemotherapy for ovarian cancer: implications for patient management and clinical trial design. J Clin Oncol. 2003 Sep. 1; 21(17):3194-200. Epub 2003 Jul. 14.

Nakazawa H, Yoshihara S, Kudo D, Morohashi H, Kakizaki I, Kon A, Takagaki K, Sasaki M. 4-methylumbelliferone, a hyaluronan synthase suppressor, enhances the anticancer activity of gemcitabine in human pancreatic cancer cells. Cancer Chemother Pharmacol. 2006 January; 57(2):165-70.

Olive P. L., Durand R. E. Drug and radiation resistance in spheroids: cell contact and kinetics. Cancer Metastasis Rev., 1994: 13: 121-138.

Ozols R F, Bundy B N, Greer B E, Fowler J M, Clarke-Pearson D, Burger R A, Mannel R S, DeGeest K, Hartenbach E M, Baergen R; Gynecologic Oncology Group. Phase III trial of carboplatin and paclitaxel compared with cisplatin and paclitaxel in patients with optimally resected stage III ovarian cancer: a Gynecologic Oncology Group study.

Park S M, Gaur A B, Lengyel E, Peter M E. The miR-200 family determines the epithelial phenotype of cancer cells by targeting the E-cadherin repressors ZEB1 and ZEB2. Genes Dev. 2008 Apr. 1; 22(7):894-907.

Ponomarev, V., Doubrovin, M., Serganova, I., Vider, J., Shavrin, A., Beresten, T., Ivanova, A., Ageyeva, L., Tourkova, V., Balatoni J., et al. A novel triple-modality reporter gene for whole-body fluorescent, bioluminescent, and nuclear noninvasive imaging. Eur. J. Nucl. Med. Mol. Imaging 2004 31, 740-751.

Simpson, R. J., Jensen, S S, Lim, J W E. Proteomics 8:4083-4099 (2008). Proteomic profiling of exosomes: Current perspectives.

Szotek P P, Pieretti-Vanmarcke R, Masiakos P T, Dinulescu D M, Connolly D, Foster R, Dombkowski D, Preffer F, Maclaughlin D T, Donahoe P K. Ovarian cancer side population defines cells with stem cell-like characteristics and Mullerian Inhibiting Substance responsiveness. Proc Natl Acad Sci USA. 2006 Jul. 25; 103(30):11154-9.

Toole B P. Hyaluronan: from extracellular glue to pericellular cue. Nat Rev Cancer. 2004 July; 4(7):528-39.

Tunggal J. K., Cowan D. S. M., Shaikh H., Tannock I. F. Penetration of anticancer drugs through solid tissue: a factor that limits the effectiveness of chemotherapy for solid tumors. Clin. Cancer Res., 1999: 5: 1583-1586

Veatch A L, Carson L F, Ramakrishnan S. Phenotypic variations and differential migration of NIH:OVCAR-3 ovarian carcinoma cells isolated from athymic mice. Clin Exp Metastasis. 1995 May; 13(3):165-72.

Wyckoff J B, Pinner S E, Gschmeissner S, Condeelis J S, Sahai E. ROCK- and myosin-dependent matrix deformation enables protease independent tumor-cell invasion in vivo. Curr Biol 2006; 16:1515-23.

Zhang S, Balch C, Chan M W, Lai H C, Matei D, Schilder J M, Yan P S, Huang T H, Nephew K P. Identification and characterization of ovarian cancer-initiating cells from primary human tumors. Cancer Res. 2008 Jun. 1; 68(11):4311-20.

1. A method to produce serosal cancer stem cells which comprises
(a) injecting an immunocompromised, non-human mammal intraperitoneally with mammalian serosal epithelial tumor cells in an amount and under conditions to produce an intraperitoneal (ip) tumor;
(b) harvesting ascites from an ip tumor-bearing, non-human mammal;
(c) fractionating the ascites into a first fraction comprising serosal catenae and leukocytes and a second fraction comprising serosal spheroids;
(d) removing the leukocytes from said first fraction to obtain a catena-enriched fraction;
(e) culturing the catena-enriched fraction for a time and under conditions to produce adherent mesenchymal cells and a suspension of serosal catenae enriched for serosal cancer stem cells.

2. The method of claim 1 which further comprises
(f) collecting said suspension of serosal catenae;
(g) separating said serosal catenae from any serosal spheroids that may have formed;
(h) serially passaging said catenae in suspension for a time and under conditions to produce a culture of free-floating serosal catenae comprising at least 50-100% serosal cancer stem cells.

3. A method to produce serosal cancer stem cells which comprises
(a) injecting an immunocompromised, non-human mammal intraperitoneally with mammalian serosal epithelial tumor cells in an amount and under conditions to produce an intraperitoneal (ip) tumor;
(b) harvesting ascites from an ip tumor-bearing, non-human mammal;
(c) fractionating the ascites into a first ascites fraction comprising serosal catenae and leukocytes and a second ascites fraction comprising serosal spheroids;
(d) culturing said second fraction for a time and under conditions to produce adherent mesenchymal cells and a suspension culture of free-floating catenae and tumor spheroids; and
(e) fractionating the suspension culture into a first culture fraction comprising free-floating catenae enriched for serosal cancer stem cells and a second culture fraction comprising free-floating tumor spheroids enriched for serosal cancer stem cells.

4. The method of claim 3, which further comprises
(f) culturing said second culture fraction for a time and under conditions to produce a further suspension culture of free-floating catenae and tumor spheroids;
(g) fractionating said further suspension culture into free-floating catenae and tumor spheroid fractions;
(h) repeating steps (f) and (g) with the free-floating tumor spheroid fraction for a time and under conditions to produce a suspension culture of free-floating tumor spheroids comprising at least 10-30% serosal cancer stem cells.

5. A method to isolate serosal catenae which comprises
(a) injecting an immunocompromised, non-human mammal intraperitoneally with mammalian serosal epithelial tumor cells in an amount and under conditions to produce an intraperitoneal (ip) tumor;
(b) harvesting ascites from an ip tumor-bearing, non-human mammal;
(c) fractionating the ascites into a first fraction comprising serosal catenae and leukocytes and a second fraction comprising serosal spheroids; and
(d) removing the leukocytes from said first fraction to obtain a catena-enriched fraction.

6. A method to isolate serosal spheroids which comprises
(a) injecting an immunocompromised, non-human mammal intraperitoneally with mammalian serosal epithelial tumor cells in an amount and under conditions to produce an intraperitoneal (ip) tumor;
(b) harvesting ascites from an ip tumor-bearing, non-human mammal;
(c) fractionating the ascites into a first fraction comprising serosal catenae and leukocytes and a second fraction comprising serosal spheroids; and
(d) isolating said serosal spheroids.

7. The method of any one of claim 1, 3, 5 or 6, which further comprises inducing intraperitoneal inflammation, prior to, concurrent with or after injection of said cells for a period sufficient to produce an ip tumor.

8. The method of claim 1, wherein said non-human mammal is a mouse lacking T cells, B cells and/or Natural Killer cells.

9. (canceled)

10. The method of any one of claim 1, 3, 5 or 6 wherein fractionating comprises filtering said ascites through a 30-60 μm filter to obtain a flow-through fraction comprising said serosal catenae and leukocytes and a retained fraction comprising serosal spheroids.

11. The method of any one of claim 1, 3, 5 or 6, wherein serosal is ovarian.

12. Isolated, clonally pure, serosal cancer stem cells.

13. A clonally pure, self-renewing population of serosal cancer stem cells comprising symmetrically dividing, free-floating chains of cells, wherein said chains comprise from about four (4) to about seventy-two (72) cells, or more, are surrounded by a glycocalyx comprising hyaluronan, and wherein said cells are E-cadherin negative, have increased engraftment potential relative to serosal epithelial tumor cells, retain serial recloning potential, and exhibit at least 50% recloning capacity in vitro.

14. The serosal cancer stem cells of claim 12 or 13, wherein said cells are ovarian cancer stem cells.

15. A method to screen a test compound for anti-proliferative effects and/or morphological effects on serosal cancer stem cells which comprises
(a) culturing any one or more of dissociated serosal catena cells, dissociated serosal spheroid cells and dissociated serosal cancer adherent cells, said cells capable of fluorescence or luminescence;
(b) contacting said cells with said test compound;
(c) detecting whether said cells proliferate catenae, spheroids and adherent cells by detecting the fluorescence or luminescence emitted by said cultures; and
(d) determining whether said test compound inhibits proliferation of said catenae, spheroids or adherent cells and/or whether the test compound alters the morphology of said catenae, spheroid or adherent cells.

16. The method of claim 15 which further comprises
(e) determining if the test compound differentially inhibits proliferation of said catenae relative to spheroids or adherent cells.

17. (canceled)

18. (canceled)

19. A method to screen a test compound for anti-proliferative or morphological effects which comprises
(a) dissociating serosal catenae and preparing a homogenous population of single cells;
(b) seeding and culturing said cells for a time and under conditions to produce catenae with an established glycocalyx coat;
(c) contacting said culture with at least one test compound for a time sufficient to allow untreated cultures to proliferate without reaching confluency; and
(d) determining whether the test compound inhibits proliferation of said catenae or alters morphology of said catenae in the treated culture.

20. The method of claim 19 wherein said culture is contacted with said test compound from about three, four, five, six or seven days after seeding.

21. The method of claim 19 which further comprises, following step (b) but prior to step (c), incubating said culture for a time and with an amount of a hyaluronidase, a collagenase or both, sufficient to remove or disrupt the glycocalyx coat of said catenae.

22. (canceled)

23. The method of claim 19 determining proliferation effect of a compound is by manually counting cells with or without staining, measuring a fluorescent signal, a luminescent signal or by alamarBlue staining and detection.

24. The method of claim 19 culturing is conducted in 384-well or 1536-well plates to allow high through put screening.

25. A method to screen a test compound for anti-proliferative or morphological effects which comprises
(a) dissociating serosal spheroids and preparing a homogenous population of single cells;
(b) seeding and culturing said cells for a time and under conditions to produce spheroids of sufficient number and size and with an established glycocalyx coat;
(c) contacting said culture with at least one test compound for a time sufficient to allow untreated cultures to proliferate without reaching confluency; and
(d) determining whether the test compound inhibits proliferation of said spheroids or alters morphology of said spheroids in the treated culture.

26. The method of claim 25 wherein said culture is contacted with said test compound from about eight to about fourteen days after seeding.

27. The method of claim 25 which further comprises, following step (b) but prior to step (c), incubating said culture for a time and with an amount of a hyaluronidase, a collagenase or both, sufficient to remove or disrupt the glycocalyx coat of said spheroids.

28. The method of claim 27, wherein said incubating time is about 10 minutes at 37° C.

29. The method of claim 25, wherein determining proliferation effect of a compound is by manually counting cell with or without staining, measuring a fluorescent signal, a luminescent signal.

30. The method of claim 25, wherein culturing is conducted in 384-well or 1536-well plates to allow high through put screening.

31. A method to treat serosal cancer in a patient undergoing chemotherapy or radiation treatment which comprises administering a hyaluronan synthase inhibitor, a hyaluronidase, a collagenase, or a combination thereof, for a time and in an amount to augment said therapy or treatment, or to improve or increase patient survival time, or to cause remission of symptoms.

32. A method to treat serosal cancer in a patient which comprises co-administering radiation treatment and a hyaluronan synthase inhibitor, a hyaluronidase, a collagenase, or a combination thereof, for a time and in an amount to cause remission of symptoms and or other measure of cancer eradication or reduction.

33. The method of claim 31 or 32, wherein any one of said hyaluronan synthase inhibitor, hyaluronidase or collagenase is PEGylated or otherwise modified to increase its half life in vivo.

34. A method to inhibit cancer stem cell self-renewal or formation in a patient which comprises administering an inhibitor of glycocalyx formation or a agent that degrades glycocalyx for a time and in an amount to said patient to inhibit glycocalyx formation or degrade the glycocalyx of CSC in the patient and to thereby inhibit self-renewal or formation of said CSC, or to cause differentiation of the CSC and make them susceptible to killing, to prevent the catenae from undergoing spheroid formation, or any combination thereof.

35. The method of claim 34, wherein said inhibitor or agent is PEGylated or otherwise modified to increase its half life in vivo.

36. An isolated nucleic acid encoding a mammalian HAS2 splice variant.

37. The nucleic acid of claim 36 having an mRNA or cDNA sequence encoding a HAS2 splice variant.

38. The nucleic acid of claim 37, wherein said nucleic acid comprises a contiguous nucleotide sequence, in 5′ to 3′ order, consisting essentially of the entirety of or a portion of exon 2 and the entirety of exon 3 of a HAS2 gene.

39. The isolated nucleic acid of claim 36, wherein said HAS2 splice variant consists essentially of amino acids 215 to 552 of a human HAS2 coding sequence.

40. A vector comprising the nucleic acid of claim 36.

41. A cell comprising the vector of claim 40.

42. An isolated nucleic acid probe for specific for detecting a mammalian HAS2 splice variant RNA or any one or more HAS2 mutations selected from the mutations identified in Tables 17 and 18.

43. An isolated mammalian HAS2 protein encoded by a HAS2 splice variant mRNA or corresponding cDNA.

44. An isolated HAS2 protein encoded by the nucleic acid of claim 36.

45. An isolated nucleic acid encoding a mammalian mutant HAS2 or a mammalian mutant HAS2 splice variant.

46. A vector comprising the nucleic acid of claim 45.

47. A cell comprising the vector of claim 46.

48. A method of monitoring and/or staging serosal cancer in a subject which comprises
(a) preparing catenae from ascites obtained from a cancer patient;
(b) detecting whether said catenae have one or more HAS2 mutations and/or express one or more HAS2 splice variants; and
(c) correlating said mutations and/or variants with the presence and/or progression of cancer in a said patient.

49. A method to identify or monitor for the presence of serosal cancer stem cells in a patient sample which comprises
(a) obtaining a cellular sample from a patient;
(b) optionally, depleting said sample of leukocytes;
(c) preparing DNA, RNA or both from the remainder of the sample;
(d) detecting whether said DNA, RNA or both has a HAS2 mutation or expresses a HAS2 splice variant, wherein identification of a mutation or a splice variant indicates the presence of serosal cancer stem cells in said sample.

50. The method of claim 49 which further comprises quantitating the amount of DNA, RNA or both having a HAS2 mutation or expressing a HAS2 splice variant, and correlating said amounts with the presence of cancer and/or progression of cancer in said patient.

51. A method to identify and/or monitor for the presence of serosal cancer stem cells in a patient sample which comprises
(a) obtaining a cellular sample from a patient;
(b) depleting the sample of leukocytes ;
(c) reacting the sample with a panel of detectable surface antigen antibodies;
(d) sorting the reacted cells into single- or multi-cell samples; and
(e) detecting whether any of said single- or multi-cell samples are positive for the presence of CD49f, CD90, CD166, PDGFRA, and GM2 proteins and negative for the presence of CD34, CD133, MUC16 and EPCAM proteins, wherein the presence and absence of said proteins identifies the reacted cells as containing serosal cancer stem cells or identifies a single cell as a serosal cancer stem cell.

52. (canceled)

53. A method to identify and/or monitor for the presence of serosal cancer stem cells in a patient sample which comprises
(a) obtaining a cellular sample from a patient;
(b) depleting the sample of leukocytes;
(c) extracting RNA from the remainder of the sample;
(d) analyzing the RNA for expression levels of a human mRNA transcriptome; and
(e) identifying samples having a surfaceome-related catena gene signature as those which have upregulated HAS2 and PDGFRA, downregulated MUC16 and EPCAM and have upregulated at least 7 additional genes listed in Table 11, wherein having those characteristics indicates the patient sample contains serosal cancer stem cells.

54. A method to identify and/or monitor for the presence of serosal cancer stem cells in a patient sample which comprises
(a) obtaining an integral membrane protein fraction from a cellular sample of a patient, wherein the cellular sample has optionally been depleted of leukocytes;
(b) analyzing the protein content of said membrane fraction by mass spectrometry;
(c) identifying samples having a surfaceome-related catena protein signature as those samples in which the spectral data indicate the presence of at least 40 proteins listed in Table 16, wherein presence of those proteins indicates the patient sample contains serosal cancer stem cells.

55. (canceled)

56. A method to identify and/or monitor for the presence of serosal cancer stem cells in a patient sample which comprises
(a) obtaining a cellular sample from a patient;
(b) depleting the sample of leukocytes;
(c) extracting RNA from the remainder of the sample;
(d) analyzing the RNA for expression levels of human miRNA; and
(e) identifying samples having an miRNA-related catena signature as those which have downregulated let-7 and 200 families of miRNA, downregulated hsa-miR-23b and hsa-miR-27b, and have upregulated at least 4 additional miRNA listed in Table 8, wherein having those characteristics indicates the patient sample contains serosal cancer stem cells.

57. A method to identify and/or monitor for the presence of serosal cancer stem cells in a patient sample which comprises
(a) obtaining a cellular sample from a patient;
(b) depleting the sample of leukocytes;
(c) extracting RNA from the remainder of the sample;
(d) analyzing the RNA for expression levels of a human mRNA transcriptome; and
(e) identifying samples having a catena gene signature as those samples which have upregulated HAS2 and PDGFRA and have upregulated at least 5 additional genes listed in Table 5, wherein having those characteristics indicates the patient sample contains serosal cancer stem cells.

58. A method to identify and/or monitor for the presence of serosal cancer stem cells in a patient sample which comprises
(a) obtaining a cellular sample from a patient;
(b) optionally, depleting the sample of leukocytes;
(c) extracting RNA from the remainder of the sample;
(d) analyzing the RNA for expression levels of a human mRNA transcriptome; and
(e) identifying samples having a catena cluster-defining gene signature as those samples which have upregulated at least six of the nine genes in LIST1 of Table 7 and have upregulated at least 5 of the genes in LIST2 of Table 7, wherein having a catena cluster-defining gene signature indicates the patient sample contains serosal cancer stem cells.

59. A method of identifying serosal cancer stem cells in a subject which comprises
(a) detecting the level of expression of ten or more genes from Table 5 in a tissue sample, wherein increased or decreased expression of the genes in accordance with Table 5 and relative to expression in serosal mesenchymal monolayer cells is indicative of the presence of serosal cancer stem cells.

60. A method to identify and/or monitor for the presence of serosal cancer stem cells in a patient sample which comprises
(a) obtaining isolated exosomes from a patient sample;
(b) analyzing the protein content of said exosomes by mass spectrometry, by antibody binding or otherwise;
(c) identifying samples having an exosomal catena protein signature as those samples in which the spectral data or other data indicate the presence of CD63, COL1A2 and at least 5 additional proteins listed in Table 13, wherein presence of said proteins indicates the patient sample contains serosal cancer stem cells.

61. A method to identify and/or monitor for the presence of serosal cancer stem cells in a patient sample which comprises
(a) obtaining isolated exosomes from a patient sample;
(b) reacting said exosomes with one or more antibodies specific for CD63, COL1A2 and at least 5 additional proteins listed in Table 13; and
(c) identifying samples having an exosomal catena protein signature as those samples in which are positive for the presence of CD63, COL1A2 and at least 5 additional proteins listed in Table 13, wherein presence of said proteins indicates the patient sample contains serosal cancer stem cells.

62. A method to identify and/or monitor for the presence of serosal cancer stem cells in a patient sample which comprises
(a) obtaining a supernatant fraction from a patient sample from which cells, cellular debris and exosomes have been removed;
(b) analyzing the protein content of said supernatant fraction by mass spectrometry;
(c) identifying samples having a secretome catena protein signature as those samples in which the spectral data indicate the presence of at least 20 proteins listed in Table 15, wherein presence of those proteins indicates the patient sample contains serosal cancer stem cells.

63. A method to identify and/or monitor for the presence of serosal cancer stem cells in a patient sample which comprises
(a) obtaining a supernatant fraction from a patient sample from which cells, cellular debris and exosomes have been removed;
(b) analyzing the protein content of said supernatant fraction by mass spectrometry;
(c) identifying samples having a glycocalyx signature as those samples in which the spectral data indicate the presence of at least 6 proteins found in glycocalyx as listed in Table 4 and the absensce of ELN, FN1 and at least 2 protein downregulated in catena as listed in Table 4, wherein presence and absence of those proteins indicates the patient sample contains serosal cancer stem cells.

64. A method to identify and/or monitor for the presence of serosal cancer stem cells in a patient sample which comprises
(a) obtaining a cellular sample or a cell lysate from a cellular sample from a patient, wherein said sample has been depleted of leukocytes;
(b) incubating said sample or said lysate with a panel of human tyrosine kinase receptor- specific antibodies and a pan-phosphotyrosine antibody; and
(c) detecting whether said sample or lysate is positive for activated phosphoproteins selected from the group consisting of PDGFRA and at least 6 of the proteins selected from the group consisting of PDGFRβ, EGFR, ERBB4, FGFR2, FGFR3, Insulin-R, IGF1R, DTK/TYRO3, MER/MERTK, MSPR/RON, Flt-3, c-rRET, ROR1, ROR2, Tie-1, Tie-2, TrkA/NTRK1, VEGFR3, EphA1, EphA3, EphA4, EphA7, EphB2, EphB4, and EphB6, wherein the detection of said activated phosphoproteins identifies the patient sample as containing serosal cancer stem cells.

65. A method to identify and/or monitor for the presence of serosal cancer stem cells in a patient sample which comprises
(a) obtaining a supernatant fraction from a patient sample from which cells and cellular debris have been removed;
(b) reacting the sample with an anti-COL1A2 antibody;
(c) detecting whether said antibody binds a low molecular weight complex of hyaluronan and collagen of less than 20,000 Daltons, wherein the detecting said complex indicates that said sample contains serosal cancer stem cells.

66. (canceled)

67. (canceled)

68. (canceled)

69. (canceled)

70. A method to screen for a metastatic inhibitor or a metastatic effector which comprises (a) intravenously injecting an immunocompromised, non-human mammal with a preparation of catenae or catena cells, (b) administering one or more test compounds to said mammal, wherein administering can be done before, after or simultaneous with injecting, and (c) assessing the time course of tumor production and/or tumor location in said mammal to that of a control mammal, to thereby identify compounds which inhibit metastasis of catena cells.

71. The method of claim 70, wherein reduction in tumor production or changes in tumor locations identifies said one or more compounds as metastasic inhibitor or a metastasic effector.

72. An in vivo method to screen for drug efficacy which comprises
(a) intraperitoneally injecting an immunocompromised, non-human mammal with a preparation of catenae or catena cells;
(b) administering one or more test compounds to said mammal, wherein administering can be done before, after or simultaneous with injecting; and
(c) assessing
(i) the time course of tumor production in said mammal,
(ii) the time course of serosal fluid production in said mammal,
(iii) the morphology of tumors in said mammal, and/or
(iv) the quantity of and/or time course of production of serosal cancer stem cells in the ascites of said mammal,
to that of a control mammal and to thereby determine the potential or actual efficacy of a drug compound in treating serosal cancer.

73. A method to produce spheroids from primary serosal tumor-derived catenae or from metastatic tumor cells which comprises culturing a suspension of said catenae or said cells for a time in a first serum-containing media containing an amount of Matrigel sufficient to induce spheroid formation and to produce a spheroid culture system, and periodically supplementing said culture system with serum-containing media without additional Matrigel.

74. (canceled)

75. A method to produce catenae from serosal fluid which comprises (a) obtaining a sample of serosal fluid from a cancer patient, (b) harvesting the cells from said fluid, (c) culturing said cells in serum-containing media supplemented with cell-free serosal fluid, (d) periodically passaging the suspension culture produced by said cells into fresh serum-containing media supplemented with cell-free serosal fluid to thereby obtain catenae.

76. (canceled)

77. (canceled)

78. A PCR primer set comprising PCR primers for mammalian genes selected from the group consisting of
(a) CD49f, CD90, CD166, PDGFRA and GM2 genes;
(b) CD49f, CD90, CD166, PDGFRA, GM2, CD34, CD133, MUC16 and EPCAM genes;
(c) HAS2, PDGFRA and at least 10 of the upregulated genes listed in Table 11;
(d) HAS2, PDGFRA, MUC16, EPCAM and at least 10 of the upregulated genes listed in Table 11;
(e) the genes of at least 40 of the proteins listed in Table 16;
(f) let-7 and 200 miRNA families, hsa-miR-23b and hsa-miR-27b, and at least 4 additional miRNAs listed in Table 8;
(g) HAS2, PDGFRA and at least 5 additional genes listed in Table 5;
(h) the nine genes in LIST1 of Table 7 and at least 5 genes in LIST2 of Table 7;
(i) ten or more genes from Table 5;
(j) CD63, COL1A2 and at least 5 additional genes for the proteins listed in Table 13;
(k) the genes of at least 20 proteins listed in Table 15;
(l) the genes of at least 6 glycocalyx proteins as listed in Table 4;
(m) ELN, FN1, the genes of at least 6 glycocalyx proteins as listed in Table 4, and the genes of at least 2 proteins listed as downregulated in Table 4; and
(n) PDGFRA and the genes for at 6 of the proteins selected from the group consisting of PDGFRβ, EGFR, ERBB4, FGFR2, FGFR3, Insulin-R, IGF1R, DTK/TYRO3, MER/MERTK, MSPR/RON, Flt-3, c-rRET, ROR1, ROR2, Tie-1, Tie-2, TrkA/NTRK1, VEGFR3, EphA1, EphA3, EphA4, EphA7, EphB2, EphB4, and EphB6.

79. A method to prepare cells with a glycocalyx coat for electron microscopy which comprises
(a) aliquoting said cells onto a cationic surface adapted for use in an electron microscope;
(b) allowing said cells to settle on and adhere to said cationic surface;
(c) adding fixatives, and optionally, one or more stains, to said aliquot of cells, and incubating for a time and under conditions to fix the cells and glycocalyx; and
(d) rinsing said fixatives and stains, if used, from said surface.

80. The method of claim 31 or 32, wherein serosal is ovarian.

81. (canceled)

82. An in vitro culture method to produce catenae and spheroids which comprises
(a) growing immortalized serosal mesenchymal cancer cells in monolayer culture for a time and in a culture media to produce a suspension culture of cells;
(b) harvesting said cells from said suspension culture;
(c) transferring said harvested cells to fresh culture media and culturing said harvested cells under conditions to produce a further suspension culture; and
(d) periodically passaging said further suspension culture by repeating steps (b) and (c) and to thereby enrich for catenae and spheroids in suspension;
and which, optionally, which further comprises
(e) collecting said suspension enriched in catenae and spheroids; and
(d) separating said catenae from said spheroids.
